(19) United States
(12) Patent Application Publication
(10) Pub. No.: US 2004/0033497 A1
(43) Pub. Date: Feb. 19, 2004

(54) Polymorphisms of PD-1

(76) Inventors: Marta E. Alarcon-Riquelme, Uppsala (SE); Ludmila Prokunina, Uppsala (SE)

Correspondence Address: BOZICEVIC, FIELD & FRANCIS LLP, 200 MIDDLEFIELD RD, SUITE 200, MENLO PARK, CA, 94025, US

(21) Appl. No.: 10/219,446

(22) Filed: Aug. 13, 2002

(51) Int. Cl. C12Q 1/68; C07H 21/04

(52) U.S. Cl. 435/6; 536/23.2

(57) ABSTRACT

The present invention is based at least in part on the identification of the genomic structure of the human PD-1 gene and on the identification of polymorphic regions within the gene. Accordingly, the invention provides nucleic acids having a nucleotide sequence encoding variants of the PD-1 gene and also provides nucleic acids having a PD-1 promoter, intron, exon and 3′ UTR sequences, and expression products. The invention also provides methods for identifying specific alleles of polymorphic regions of a PD-1 gene, methods for determining whether a subject has or is at risk of developing any disease that is associated with a specific allele of a polymorphic region of a PD-1 gene, and kits for performing such methods.

This invention relates to PD-1 polymorphisms. More particularly the present invention relates to nucleic acids encoding PD-1 polymorphs, and their use for the diagnosis and treatment of diseases and conditions associated with autoimmune disorders.

Diseases and conditions associated with autoimmune disorders such as SLE, myasthenia gravis, multiple sclerosis, Type 1 diabetes, rheumatoid arthritis, Sjögrens syndrome, atopy and allergy are major health risks through the industrialised and developing world.

An autoimmune disorder is a condition in which the body creates antibodies against its own tissues.

Systemic lupus erythematosus, (SLE or lupus), is an autoimmune disorder which affects many parts of the body. A person with SLE produces antibodies against many of their own tissues. This autoimmune reaction can damage many parts of the body for example the brain and nervous system, digestive system, eyes, heart, joints and muscles, kidney, lung and skin. SLE can manifests as a single symptom or many disparate symptoms.

The cause of SLE is believed to be autoimmunogenic disorder. SLE tends to run in families and tends to be hereditary. Research suggests that autoimmune disorders may be triggered by a transfer of cells between the foetus and the mother during pregnancy. In studies which involved women with scleroderma, an autoimmune disorder involving the skin, it was shown that these women have more foetal cells in their blood decades after a pregnancy than women who don't have scleroderma. While further research is needed to substantiate these findings, the study does offer an explanation for the much higher incidence of autoimmune disorders in women especially mothers or women who have been pregnant, than in men.

Certain medications are also known to cause systemic lupus erythematosus. These include procainamide, hydralazine, isoniazid, and chlorpromazine. Events which may trigger the disease include infection, stress, exposure to toxins, and sunlight.

Women account for 80% to 90% of cases of SLE. It is more common in black women than in white women. SLE is also more common in Asian, Hispanic, and Native American women. Most cases of SLE cannot be prevented.

Currently diagnosis of SLE requires a complete medical history and physical examination and involves the use of many disparate tests, which include, ANA blood tests which identify antibodies that the person has produced against their own tissues, CT scan, chest x-ray, electrocardiogram, kidney biopsy, MRI Scan and spinal tap.

SLE can be fatal, often as a result of kidney failure, infections, or heart attack. A number of medications are used to treat SLE, for example, the following:

antimalarial medications, such as quinacrine and hydroxychloroquine. These are used to treat skin problems and arthritis,

corticosteroids, such as prednisone and methylprednisolone. These reduce the immune system response,

nonsteroidal anti-inflammatory drugs, or NSAIDs, such as ibuprofen and naproxen, these medications reduce fever and treat pain,

powerful cytotoxic medications, which kill cells, these are used to treat nephritis, a serious kidney problem.

Individuals with end-stage kidney disease may benefit from kidney dialysis or a kidney transplant. The medications used to treat lupus have significant side effects. Unfortunately, some of these side effects can mimic the symptoms of the disease itself.

The human PD-1 gene has been mapped to 2q37.3 (references 11 and 12). This gene codes for a membrane molecule containing a tyrosine-based inhibitory motif and is of importance in T-cell development and tolerance (reference 13). The cDNA encoding PD-1 has been disclosed in U.S. Pat. No. 5,629,204 and U.S. Pat. No. 5,698,520. PD-1 is expressed in humans during B and T cell development and is induced by lymphocyte activation. Cross-linking of mouse PD-1 with the ligand PD-1 L1 and PD-1 L2 induce inhibition of T-cell activation (references 15 and 16) and mice made deficient for PD-1 develop autoantibodies. However, to date there has been no correlation or disclosure that PD-1 in humans is a factor in the development of autoimmune disease and conditions such as SLE.

The present inventors have found that polymorphs of PD-1 are factors in the development of autoimmune diseases, especially human autoimmune diseases such as SLE. In particular the inventors have found that the prevalence or susceptibility of an individual or population of individuals to autoimmune diseases such as SLE is in part determined by which of the PD-1 nucleic acid polymorph sequences an individual or population of individuals possess.

### SUMMARY OF THE INVENTION

The present invention is derived from the discovery of the genomic structure of the human PD-1 gene (NCBI REF 986034, BankIT 392218 and GeneBank AF363458, unpublished) followed by the identification and sequencing of unexpected polymorphic regions within the gene, which are surprisingly associated with specific diseases, disorders or conditions, including autoimmune disorders such as myasthenia gravis, multiple sclerosis, Type 1 diabetes, rheumatoid arthritis, Sjögrens syndrome, atopy, allergy, systemic lupus erythematosus and diseases associated with SLE which include fatigue, fever, loss of appetite, nausea, weight loss, hives, loss of scalp hair, red “butterfly rash” and raised rash, sensitivity to sun, ulcers in mouth, nose, or vagina, arthritis, joint pain, loss of blood supply to bone, pain, infections within joints, decrease in kidney function including, blood, aberrant amounts of protein or white blood cells in urine, intracerebral haemorrhage, headaches, loss of coordination, memory loss, seizures, strokes, anaemia, low white blood cell or low platelet count, pericardial effusion, heart attack, inflammation in the heart, infection in the heart, inflammation of the lining of the heart, infection of the lining of the heart, heart valve problems, shortness of breath, cough, inflammation of the lungs, inflammation of the lining of the lungs, abdominal distress, diarrhoea, enlargement of the liver, loss of appetite, nausea and vomiting, blindness, visual impairment, dryness of the eyes and dryness of the mouth.

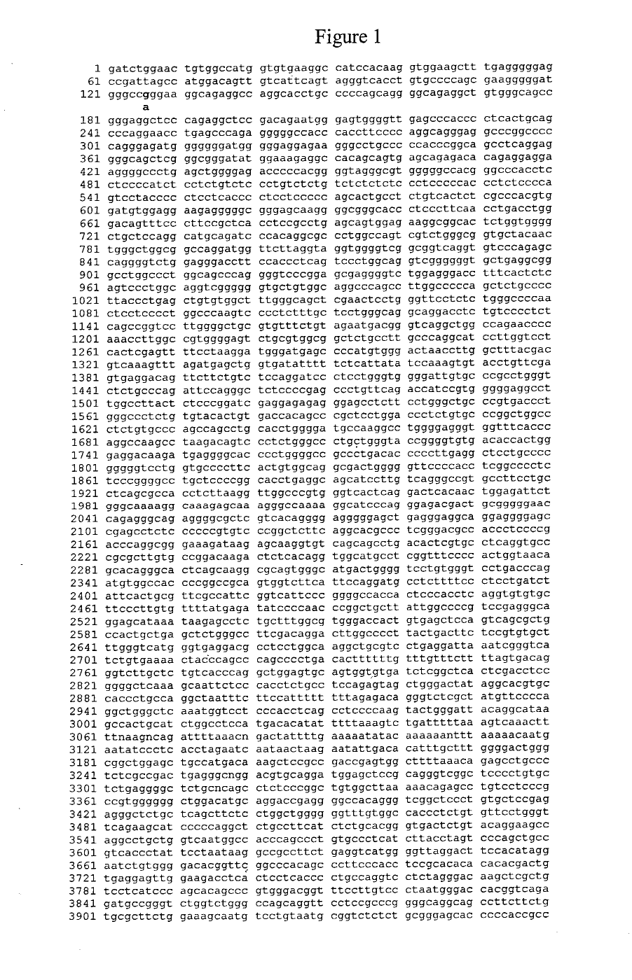

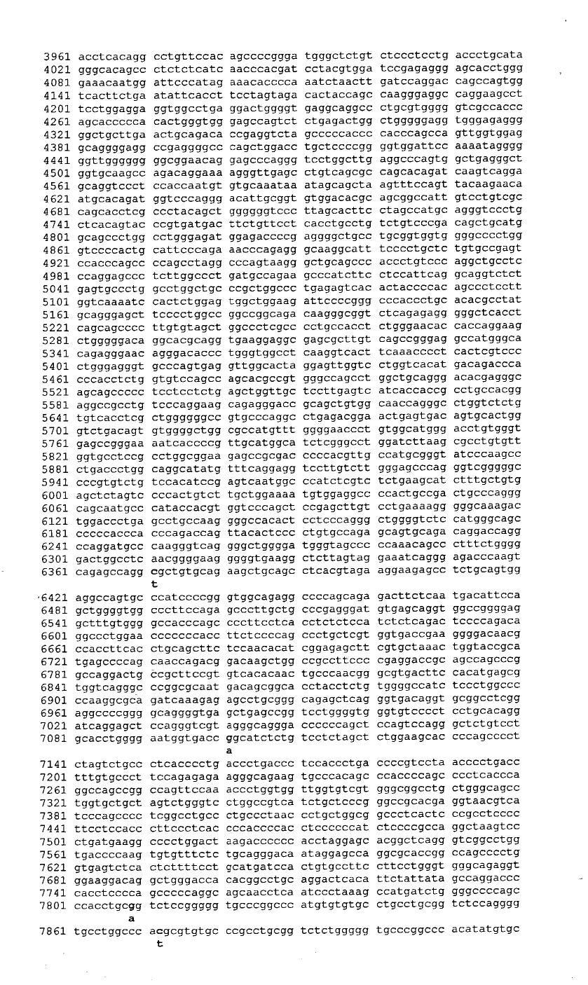

The human PD-1 gene contains a 5′ promoter region, 5 coding exons, 4 non-coding introns and a 3′UTR region. This gene unexpectedly contains at least nine polymorphic regions. The structure of the gene and the position of the 5′promoter region, exons, introns, 3′UTR and polymorphic regions are shown in to FIG. 1 and in SEQ ID N^{o} 1.

In one embodiment, the invention provides isolated or purified nucleic acids comprising an intronic sequence from a PD-1 gene that comprises the 5′ promoter region, regions of coding nucleic acids, intronic regions and 3′ UTR region of nucleic acid. In a preferred embodiment, the PD-1 gene is a human gene. In another preferred embodiment, the nucleic acid of the invention has a nucleotide sequence set forth in FIG. 1 and SEQ ID N^{o} 1, complements thereof, or homologues thereof. In yet another embodiment, the sequence of the nucleic acid is capable of hybridising under appropriate stringency to a nucleic acid having a nucleotide sequence set forth in any one of the nucleic acid sequences of SEQ ID N^{o}s 1 to 12 or complements thereof. In a further preferred embodiment the nucleic acids of the invention have at least 80% sequence identity with any one of the nucleic acid sequences of SEQ ID N^{o}s 1 to 12.

Nine polymorphic regions have been identified in the human PD-1 gene by analysing the DNA of a specific population of individuals. One polymorphism found in the population is a change from a guanine to adenine at position 126, as shown in FIG. 1 and SEQ ID N^{o}s 1, 6 or 15 and FIG. 1 and SEQ ID N^{o}s 1, 6 or 16. This polymorphism is located in the 5′ promotor region of the PD-1 gene, and results in an inhibition of binding of the Ikaros transcription factor to bind to its binding site when a guanine is replaced by an adenine.

A second polymorphism, located in intron 1 is a change from cytosine to thymine at position 6371, as shown in FIG. 1 and SEQ ID N^{o} 1 or SEQ ID N^{o} 2.

A third polymorphism located in intron 2 is a change of guanine to adenine at position 7101, as shown in FIG. 1 and SEQ ID N^{o}s 1, 3, 19 or 20.

A fourth, fifth and six polymorphism are located in intron 4 at nucleotide positions 7809, 7872 and 8162, respectively. The substitution at position 7809 is of guanine with adenine, as shown in FIG. 1 and SEQ ID N^{o} 1, 5 or SEQ ID N^{o} 26 to 29. The substitution at position 7872 is of cytosine with thymine, as shown in FIG. 1 and SEQ ID N^{o} 1, 5 or SEQ ID N^{o}s 26 to 29. The substitution at position 8162 is of guanine with adenine, as shown in FIG. 1 and SEQ ID N^{o} 1 and 5. This polymorphism at position 7809 in the PD-1 gene is an AML-1 transcription factor binding site, and results in an inhibition of binding of the AML-1 transcription factor to bind to its binding site when a guanine is replaced by an adenine.

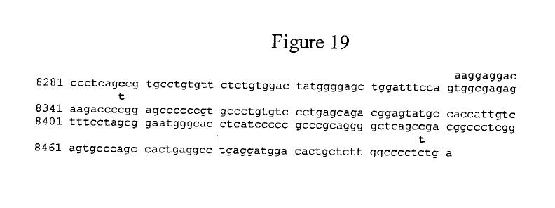

A seventh polymorphism is located in exon 5 at position 8288 as shown in FIG. 1, and FIG. 19, SEQ ID N^{o} 1 and SEQ ID N^{o} 12. This polymorphism is a change from cytosine to thymine.

An eighth polymorphism is located in exon 5 at position 8448 as shown in FIG. 1, SEQ ID N^{o} 1, SEQ ID N^{o} 12, SEQ ID N^{o} 30 or SEQ ID N^{o} 31. This polymorphism is a change from cytosine to thymine.

A ninth polymorphism is to be found 3′ to the PD-1 stop codon at position 9400, as shown in FIG. 1 and SEQ ID N^{o}s 1, 7, 33 or 34. This polymorphism is a change from guanine to adenine.

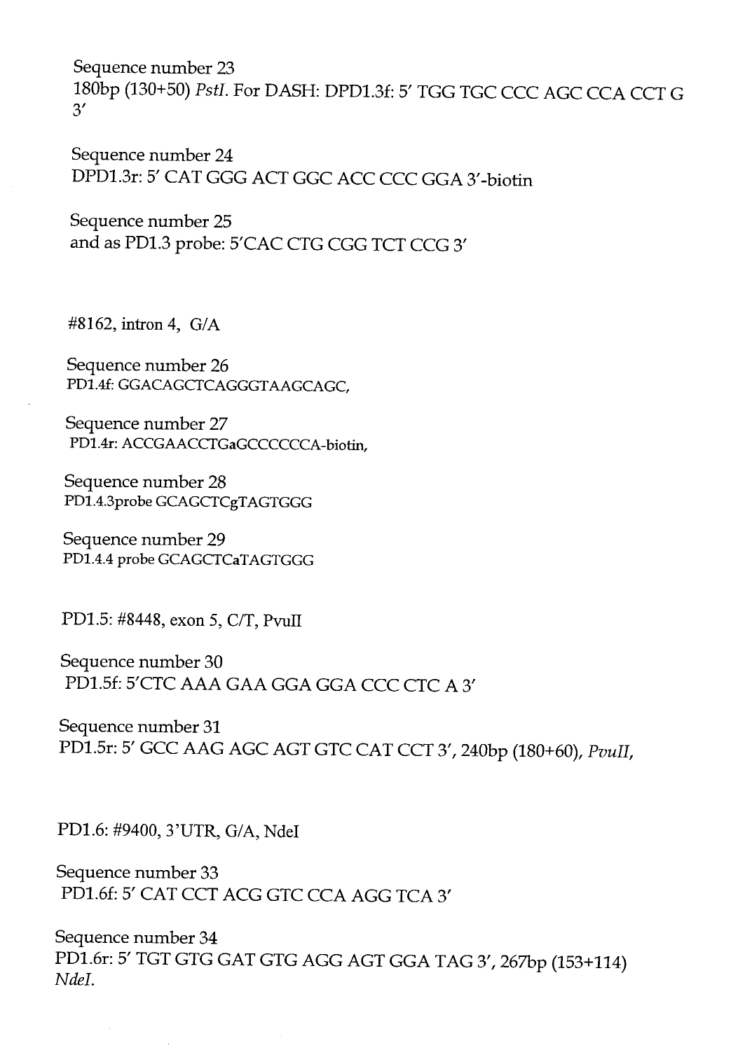

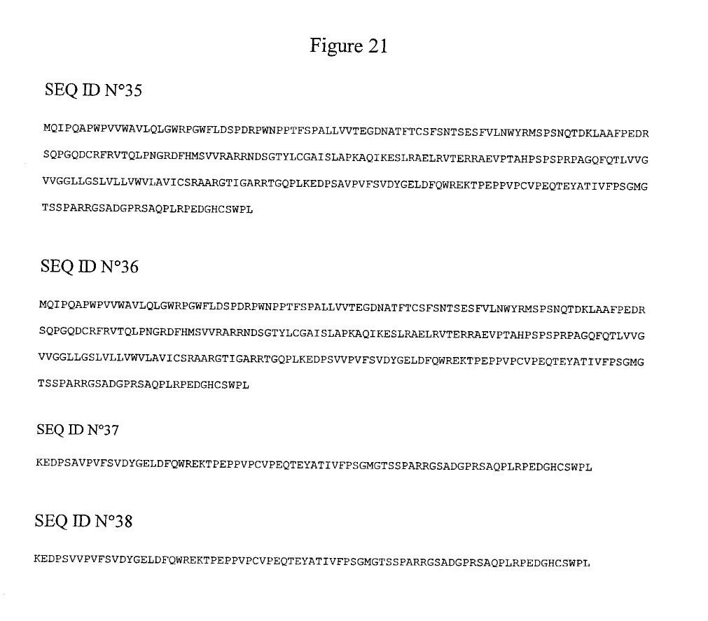

In a second embodiment, the invention provides isolated or purified expression products or fragments thereof of the PD-1 gene as shown in SEQ ID N^{o} 1. In a further preferred embodiment the polypeptides of the invention have at least 90% sequence identity with any one of the expression products of SEQ ID N^{o} 1, or fragments thereof. In a further preferred embodiment the polypeptides of the invention have at least 90% sequence identity with any one of the peptides encoded by SEQ ID N^{o}s 35 to 38, or fragments thereof, as shown in FIG. 21.

In a third embodiment the nucleic acids of the invention can be used, in prognostic and/or diagnostic methods. The nucleic acids of the invention can be used as probes or primers to determine whether a subject has or is at risk of developing a disease or disorder associated with a specific allelic variant of a PD-1 polymorphism, for example, a disease or disorder associated with an aberrant PD-1 activity.

In a fourth embodiment the nucleic acids of the invention can be used in the treatment of diseases associated with aberrant PD-1 function.

In a fifth embodiment the expression products of nucleic acids of the invention can be used, in prognostic and/or diagnostic methods. The expression products of the invention can be used as probes to determine whether a subject has or is at risk of developing a disease or disorder associated with a specific allelic variant of a PD-1 polymorphism, for example, a disease or disorder associated with an aberrant PD-1 activity.

In a sixth embodiment the expression products of nucleic acids of the invention can be used in the treatment of diseases associated with aberrant PD-1 function.

Antibody probes that specifically bind to polymorphs of PD-1 peptide or fragments thereof are also part of the invention. Preferred polymorphs of PD-1 to which antibody probes are raised have at least 90% sequence identity any one of the peptides encoded in SEQ ID N^{o}s 35 to 38, or fragments thereof, as shown in FIG. 21.

The invention further describes vectors which encode the claimed nucleic acids; host cells transfected with said vectors whether prokaryotic or eukaryotic; and transgenic non-human animals that contain a heterologous form of a functional or non-functional PD-1 allele described herein. Such a transgenic animal can serve as an animal model for studying, for example, the effect of specific allelic variations, including mutations of a PD-1 gene, especially a human PD-1 gene or for use in drug screening or recombinant protein production.

The invention further provides methods for determining the molecular structure of at least a portion of a PD-1 gene. In a preferred embodiment, the method comprises contacting a sample nucleic acid comprising a PD-1 gene sequence with a probe or primer having a sequence which is complementary to a PD-1 gene sequence and comparing the molecular structure of the sample nucleic acid with the molecular structure of a control (known) PD-1 gene (for example, a PD-1 gene from a human not afflicted with a condition or a disease associated with an aberrant PD-1 activity). The method of the invention can be used for example in determining the molecular structure of at least a portion of an exon, an intron, a promoter, or a 3′UTR. In a preferred embodiment, the method comprises determining the identity of at least one nucleotide. In even more preferred embodiments, the nucleotide is guanine or adenine at nucleotide position 126 of the PD-1 gene, as shown in FIG. 1, cytosine or thymine at position 6371, as shown in FIG. 1, guanine or adenine at position 7101, as shown in FIG. 1, guanine or adenine at position 7809, as shown in FIG. 1, cytosine of thymine at position 7872 as shown in FIG. 1, guanine or adenine at position 8162 as shown in FIG. 1, cytosine or thymine at position 8288, as shown in FIG. 1 and SEQ ID N^{o} 1, cytosine or thymine at position 8448, as shown in FIG. 1 or guanine or adenine at position 9400, as shown in FIG. 1. In another preferred embodiment, the method comprises determining the nucleotide content of at least a portion of a PD-1 gene, such as by sequence analysis. In yet another embodiment, determining the molecular structure of at least a portion of a PD-1 gene is carried out by single-stranded conformation polymorphism. Non-limiting examples of methods within the scope of the invention for determining the molecular structure of at least a portion of a PD-1 gene include hybridisation of allele-specific oligonucleotides, sequence specific amplification, and primer specific extension.

In at least some of the methods of the invention, the probe or primer is allele specific. Preferred probes or primers are single stranded nucleic acids, which optionally are labelled.

The methods of the invention can be used for determining the identity of the allelic variant of a polymorphic region of a human PD-1 gene present in a subject. For example, the method of the invention can be useful for determining whether a subject has, or is at risk of developing, a disease or condition associated with a specific allelic variant of a polymorphic region in the human PD-1 gene. In one embodiment, the disease or condition is characterized by an aberrant PD-1 activity, such as an aberrant PD-1 protein level, which can result from an aberrant expression of a PD-1 gene. The disease or condition can be autoimmune disorders such as myasthenia gravis multiple sclerosis, Type 1 diabetes, rheumatoid arthritis, Sjögrens syndrome, atopy, allergy, systemic lupus erythematosus and diseases associated with SLE which include fatigue, fever, loss of appetite, nausea, weight loss, hives, loss of scalp hair, red “butterfly rash” and raised rash, sensitivity to sun, ulcers in mouth, nose, or vagina, arthritis, joint pain, loss of blood supply to bone, pain, infections within joints, decrease in kidney function including, blood, aberrant amounts of protein or white blood cells in urine, intracerebral haemorrhage, headaches, loss of coordination, memory loss, seizures, strokes, anaemia, low white blood cell or low platelet count, pericardial effusion, heart attack, inflammation in the heart, infection in the heart, inflammation of the lining of the heart, infection of the lining of the heart, heart valve problems, shortness of breath, cough, inflammation of the lungs, inflammation of the lining of the lungs, abdominal distress, diarrhoea, enlargement of the liver, loss of appetite, nausea and vomiting, blindness, visual impairment, dryness of the eyes and dryness of the mouth. Accordingly, the invention provides methods for predicting or diagnosing autoimmune disorders, conditions and diseases, specifically disorders conditions and diseases associated with myasthenia gravis multiple sclerosis, Type 1 diabetes, rheumatoid arthritis, Sjögrens syndrome, atopy, allergy and most specifically disorders conditions and diseases associated with SLE.

The methods of the invention can also be used in selecting the appropriate drug to administer to a subject to treat a disease or condition, such as autoimmune disorders. In fact, specific allelic variants of PD-1 polymorphic regions may be associated with a specific response to a specific drug by an individual having such an allele. For example, a specific PD-1 allele may encode a PD-1 protein having a modified affinity for certain types of molecules. Accordingly, the action of a drug necessitating interaction with a PD-1 protein will be different in individuals carrying such a PD-1 allele. Alternatively a specific PD-1 allele may encode a variant which modifies the level of protein expression.

In a further embodiment, the invention provides a method for treating a subject having a disease or condition associated with a specific allelic variant of a polymorphic region of a PD-1 gene. In one embodiment, the method comprises (a) determining the identity of the allelic variant; and (b) administering to the subject a compound that compensates for the effect of the specific allelic variant. In a preferred embodiment, the specific allelic variant is a mutation. The mutation can be located, for example, in a promoter region, an intron, or an exon of the gene, or in the 3′UTR of the gene. In one embodiment, the compound has an antagonistic or agonistic effect or other modulatory effect on PD-1 protein levels which prevents or alleviates autoimmune disorders such as myasthenia gravis multiple sclerosis, Type 1 diabetes, rheumatoid arthritis, Sjögrens syndrome, atopy, allergy, systemic lupus erythematosus and diseases associated with SLE which include fatigue, fever, loss of appetite, nausea, weight loss, hives, loss of scalp hair, red “butterfly rash” and raised rash, sensitivity to sun, ulcers in mouth, nose, or vagina, arthritis, joint pain, loss of blood supply to bone, pain, infections within joints, decrease in kidney function including, blood, aberrant amounts of protein or white blood cells in urine, intracerebral haemorrhage, headaches, loss of coordination, memory loss, seizures, strokes, anaemia, low white blood cell or low platelet count, pericardial effusion, heart attack, inflammation in the heart, infection in the heart, inflammation of the lining of the heart, infection of the lining of the heart, heart valve problems, shortness of breath, cough, inflammation of the lungs, inflammation of the lining of the lungs, abdominal distress, diarrhoea, enlargement of the liver, loss of appetite, nausea and vomiting, blindness, visual impairment, dryness of the eyes and dryness of the mouth. In a preferred embodiment, the compound is selected from the group consisting of a nucleic acid, a protein, a peptidomimetic, or a small molecule. For example, if a subject has the allele of residue 126 of the PD-1 gene resulting in a predisposition for SLE and associated disorders, the disorders may be prevented from occurring or may be reduced, by administering to the subject a pharmaceutically effective amount of a compound which provides compensation for the dysfunction caused by aberrant PD-1 function, especially where caused by an allelic variant.

The invention also provides substantially purified nucleic acids and oligonucleotides which can be used as a therapy to treat diseases and conditions associated with PD-1. Preferred nucleic acids are any one of SEQ ID N^{o}s 1 to 34.

The invention also provides probes and primers comprising substantially purified oligonucleotides, which correspond to a region of nucleotide sequence which hybridises to at least 10 consecutive nucleotides of the sequence set forth in any one of SEQ ID N^{o}s 1, 2, 3, 4, 5, 7, 8, 9, 10 11 or 12 or to any one of the complementary sequences set forth as SEQ ID N^{o}s 1, 2, 3, 4, 5, 6, 7, 8, 9, 10, 11 or 12. In preferred embodiments, the probe/primer further includes a label group attached thereto, which is capable of being detected.

In another embodiment, the invention provides a kit for amplifying and/or for determining the molecular structure of at least a portion of a PD-1 gene, comprising a probe or primer capable of hybridising to a PD-1 gene and instructions for use. In one embodiment, the probe or primer is capable of hybridising to or amplifying a PD-1. In another embodiment, the probe or primer is capable of hybridising to or amplifying an allelic variant of a PD-1. In a preferred embodiment, the polymorphic regions encode guanine or adenine at nucleotide position 126 of the PD-1 gene, cytosine or thymine at position 6371, guanine or adenine at position 7101, guanine or adenine at position 7809 cytosine of thymine at position 7872, guanine or adenine at position 8162, cytosine or thymine at position 8288, cytosine or thymine at position 8448, or guanine or adenine at position 9400. In a preferred embodiment, determining the molecular structure of a region of a PD-1 gene comprises determining the identity of the allelic variant of the polymorphic region.

A kit of the invention can be used, for example, for determining whether a subject has or is at risk of developing a disease associated with a specific allelic variant of a polymorphic region of a PD-1 gene. In a preferred embodiment, the invention provides a kit for determining whether a subject has or is at risk of developing a disease or condition associated with autoimmune disorders such as multiple sclerosis, myasthenia gravis Type 1 diabetes, rheumatoid arthritis, Sjögrens syndrome, atopy, allergy, systemic lupus erythematosus and diseases associated with SLE. The disease or condition can be associated with an aberrant PD-1 activity, which can result, for example, from a mutation in the PD-1 gene. The kit of the invention can also be used in selecting the appropriate drug to administer to a subject to treat a disease or condition, such as a disease or condition set forth above. In fact, pharmacogenetic studies have shown that the genetic background of individuals plays a role in determining the response of an individual to a specific drug. Thus, determining the allelic variants of PD-1 polymorphic regions of an individual can be useful in predicting how an individual will respond to a specific drug, for example, a drug for treating a disease or disorder associated with an aberrant PD-1 activity and/or a autoimmune disorders such as multiple sclerosis, Type 1 diabetes, rheumatoid arthritis, Sjögrens syndrome, atopy, allergy, systemic lupus erythematosus associated aberrant PD-1.

The inventors have identified nucleic acid sequences associated with autoimmune diseases in humans, most notably SLE (NCBI REF 986034, BankIt Ref 392218 and GeneBank AF363458, Unpublished). Furthermore, the inventors have identified polymorphisms in the gene that make a subject more susceptible or prone to such conditions.

The present invention is based at least in part on the discovery of the genomic structure of the human PD-1 gene and on the identification of polymorphic regions within the gene which correlate with specific diseases or conditions, including autoimmune disorders such as myasthenia gravis multiple lo sclerosis, Type 1 diabetes, rheumatoid arthritis, Sjögrens syndrome, atopy, allergy, systemic lupus erythematosus and diseases associated with SLE which include fatigue, fever, loss of appetite, nausea, weight loss, hives, loss of scalp hair, red “butterfly rash” and raised rash, sensitivity to sun, ulcers in mouth, nose, or vagina, arthritis, joint pain, loss of blood supply to bone, pain, infections within joints, decrease in kidney function including, blood, aberrant amounts of protein or white blood cells in urine, intracerebral haemorrhage, headaches, loss of coordination, memory loss, seizures, strokes, anaemia, low white blood cell or low platelet count, pericardial effusion, heart attack, inflammation in the heart, infection in the heart, inflammation of the lining of the heart, infection of the lining of the heart, heart valve problems, shortness of breath, cough, inflammation of the lungs, inflammation of the lining of the lungs, abdominal distress, diarrhoea, enlargement of the liver, loss of appetite, nausea and vomiting, blindness, visual impairment, dryness of the eyes and dryness of the mouth.

As shown in FIG. 1, the human PD-1 gene is at least 9625 base pairs long and has 5 coding exons and 4 introns. The exons are numbered 1 to 5 from 5′ to 3′ and the introns are numbered 1 through 4 from 5′ to 3′. 5′ of Exon 1, nucleic acids 1 to 663 encode a promoter region, as shown in FIG. 1 and SEQ ID N^{o} 1 or 6. 3′ to Exon 5, nucleic acids 8512 to 9625 is the 3′ UTR, as shown on FIG. 1 and SEQ ID N^{o} 1 or 7.

Exon 1 corresponds to the first Exon, nucleic acids 664 to 807, as shown in FIG. 1 and SEQ ID N^{o} 1 or SEQ ID N^{o} 8 is situated 3′ of the promoter and 5′ to intron 1 on the sense coding strand of the gene, and contains the initiation codon. Intron 1, nucleic acids 808 to 6588 is situated immediately downstream of exon 1, as shown in FIG. 1, SEQ ID N^{o} 1 or SEQ ID N^{o} 2 on the sense coding strand of the gene. Exon 2, nucleic acids 6589 to 6948, as shown in FIG. 1, SEQ ID N^{o} 1 or SEQ ID N^{o} 9 is situated 3′ to intron 1 and 5′ to intron 2 on the sense coding strand of the gene. Intron 2, nucleic acids 6949 to 7215, as shown in FIG. 1, SEQ ID N^{o} 1 or SEQ ID N^{o} 3 is situated 3′ with respect to exon 2 and 5′ with respect to exon 3 on the sense coding strand of the gene. Exon 3 nucleic acids 7216 to 7371 as shown in FIG. 1, SEQ ID N^{o} 1 or SEQ ID N^{o} 10 is situated 3′ with respect to intron 2 and 5′ with respect to intron 3 on the sense coding strand of the gene. Intron 3 nucleic acids 7372 to 7585 as shown in FIG. 1 SEQ ID N^{o} 1 or SEQ ID N^{o} 4 is situated 3′ with respect to exon 3 and 5′ with respect to exon 4 on the sense coding strand of the gene. Exon 4 nucleic acids 7586 to 7620 as shown in FIG. 1, SEQ ID N^{o} 1 or SEQ ID N^{o} 11 is located 3, with respect to intron 3 and 5′ with respect to intron 4 on the sense coding strand of the gene. Intron 4 nucleic acids 7621 to 8271 as shown in FIG. 1, SEQ ID N^{o} 1 or SEQ ID N^{o} 5 is located 3′ with respect to exon 4 and 5′ with respect to exon 5 on the sense coding strand of the gene. Exon 5 nucleic acids 8272 to 8511, as shown in FIG. 1, SEQ ID N^{o} 1 or SEQ ID N^{o} 12 is located 3′ with respect to intron 4 and 5′ with respect to the 3′UTR.

An analysis of human individuals and families suffering from autoimmune conditions diseases or disorders particularly SLE, indicated that the disease locus was 2q37 and that the gene was PD-1. Further analysis indicated that this gene has at least nine polymorphisms. Several of these polymorphisms were associated with autoimmune diseases, conditions or disorders, particularly SLE and nephritis.

One polymorphism found in the population is a change from a guanine at position 126, as shown in FIG. 1 and SEQ ID N^{o}s 1, 6 or 15 to an adenine as shown in FIG. 1 and SEQ ID N^{o}s 1, 6 or 16. This polymorphism is located in the 5′ promotor region of the PD-1 gene and comprises an Ikaros transcription factor binding site 126 to 130 bp GGGM—binding site, (ref: Molnar A., Georgöpulos, K., Mol. Cel. Biol., 14: 8292-8303, 1994) as shown in SEQ ID N^{o} 1. Replacement of guanine for adenine disrupts the binding of the transcription factor Ikaros for the binding site.

A second polymorphism, located in intron 1 is a change from cytosine to 1o thymine at position 6371, as shown in FIG. 1 and SEQ ID N^{o} 1 or SEQ ID N^{o} 2.

A third polymorphism located in intron 2 is a change of guanine to adenine at position 7101, as shown in FIG. 1 and SEQ ID N^{o}s 1, 3, 19 or 20.

A fourth, fifth and six polymorphism is located in intron 4 at nucleotide positions 7809, 7872 and 8162 respectively. The substitution at position 7809 is of guanine with adenine, as shown in FIG. 1 and SEQ ID N^{o} 1, 5 or SEQ ID N^{o} 26 to 29. The substitution at position 7872 is of cytosine with thymine, as shown in FIG. 1 and SEQ ID N^{o} 1, 5 or SEQ ID N^{o} 26 to 29. The substitution at position 8162 is of guanine with adenine, as shown in FIG. 1 and SEQ ID N^{o} 1 or 5. The inventors have shown this nucleotide 7809 TGCGGT comprise an AML1 transcription factor binding site in the PD-1 gene, as shown in FIG. 1, SEQ ID N^{o} 1, SEQ ID N^{o} 5 (7806 to 7811 (tgcg/agt). Substitution of guanine with adenine results in an inhibition of binding of the AML-1 transcription factor to bind to its binding site (ref: Meyers S., Downing J. R., Hiebert S. W., Mol. Cell Biol. 13: 6336-6345, 1993).

A seventh polymorphism is located in exon 5 at position 8288 as shown in FIG. 1, SEQ ID N^{o} 1 or SEQ ID N^{o} 12. This polymorphism is a change from cytosine to thymine, which changes the codon for alanine at amino acid 215 to a codon encoding valine. This modification is close to the PD-1 cytoplasmic domain ITIM motif.

An eighth polymorphism is located in exon 5 at position 8448 as shown in FIG. 1, SEQ ID N^{o} 1 or SEQ ID N^{o} 12. This polymorphism is a change from cytosine to thymine.

An ninth polymorphism is to be found 3′ to the PD-1 stop codon at position 9400, as shown in FIG. 1 and SEQ ID N^{o}s 1, 7, 33 or 34. This polymorphism is a change from guanine to adenine.

Furthermore, the inventors have identified at least one NFκB transcription factor binding site in intron 4, as shown in SEQ ID N^{o} 1 or SEQ ID N^{o} 5 (7817-7825 GGGGTGCCC) and that binding of the transcription factor NFκB to this site In is not perturbed by the indicated polymorphisms at positions 7809, 7872 or 8162.

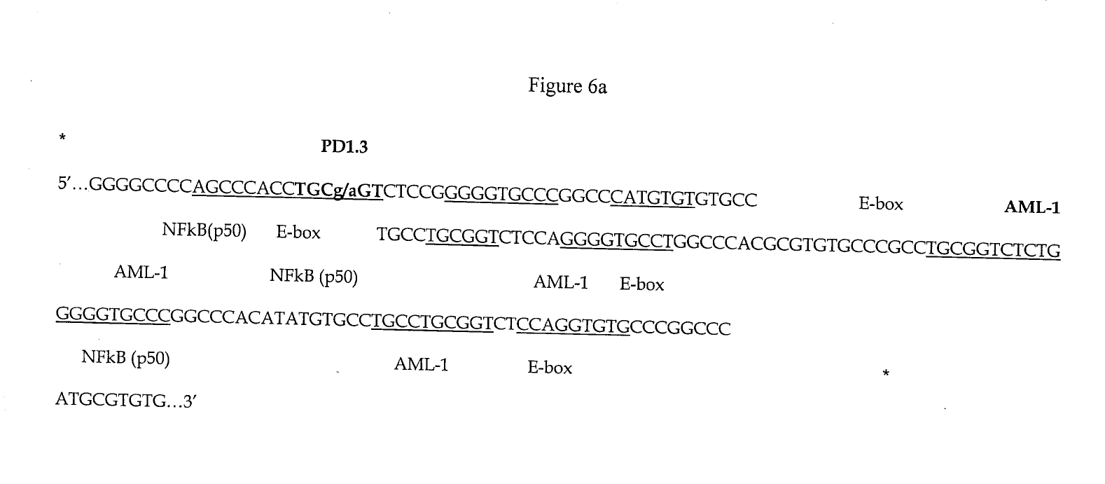

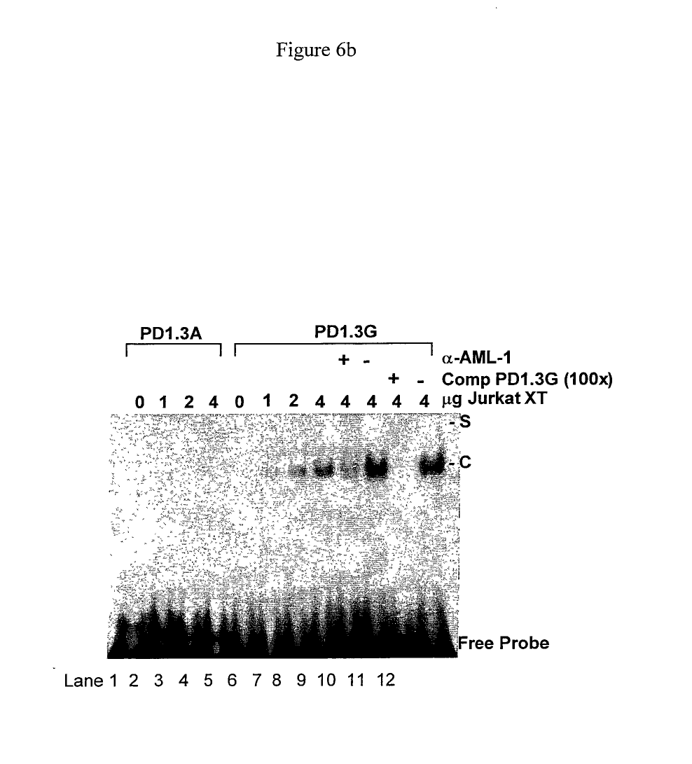

Moreover the inventors have identified at least one E-box transcription factor binding site in intron 4, as shown in SEQ ID N^{o} 1 or SEQ ID N^{o} 5 and FIG. 6 and that binding of the transcription factor NFκB to this site is not perturbed by the indicated polymorphisms at positions 7809, 7872 or 8162.

Accordingly, the invention provides nucleic acids, for example, intronic sequences, useful as probes or primers for determining the identity of an allelic variant of a PD-1 polymorphic region. The invention also provides methods for determining the identity of the alleles of a specific polymorphic region of a PD-1 gene. Such methods can be used, for example, to determine whether a subject has or is at risk of developing a disease or condition associated with one or more specific alleles of polymorphic regions of a PD-1 gene. In a preferred embodiment, the disease or condition is caused or contributed to by an aberrant PD-1 bioactivity. Other aspects of the invention are described below or will be apparent to one of skill in the art in light of the present disclosure.

For convenience, the meaning of certain terms and phrases employed in the specification, examples, and appended claims are provided below.

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

The term “allelic variant of a polymorphic region of an PD-1 gene” refers to a region of a PD-1 gene having one of several nucleotide sequences found in that region of the gene in other individuals.

Antigenic functions include possession of an epitope or antigenic site that is capable of cross-reacting with antibodies raised against a naturally occurring or denatured PD-1 polypeptide or fragment thereof.

Biologically active PD-1 polypeptides include polypeptides having both an effector and antigenic function, or only one of such functions. PD-1 polypeptides include antagonist polypeptides and native PD-1 polypeptides, provided that such antagonists include an epitope of a native PD-1 polypeptide. An effector function of PD-1 polypeptide can be the ability to bind to a ligand.

As used herein the term “bioactive fragment of a PD-1 protein” refers to a fragment of a full-length PD-1 protein, wherein the fragment specifically mimics or antagonizes the activity of a wild-type PD-1 protein. The bioactive fragment preferably is a fragment capable of binding to a second molecule, such as a ligand.

The term “exon”, “exonic sequence” or “exonic nucleotide sequence” refers to the nucleotide sequence of an exon or portion thereof.

The term “an aberrant activity” or “abnormal activity”, as applied to an activity of a protein such as PD-1, refers to an activity which differs from the activity of the wild-type or native protein or which differs from the activity of the protein in a healthy subject, for example, a subject not afflicted with a disease associated with a specific allelic variant of an PD-1 polymorphism. An activity of a protein can be aberrant because it is stronger than the activity of its native counterpart. Alternatively, an activity can be aberrant because it is weaker or absent related to the activity of its native counterpart. An aberrant activity can also be a change in an activity. For example an aberrant protein can interact with a different protein relative to its native counterpart. A cell can have an aberrant PD-1 activity due to over expression or under expression of the gene encoding PD-1. An aberrant PD-1 activity can a protein which has greater or less activity that its wild type counterpart. An aberrant PD-1 activity can also result from a lower or higher level of PD-1 on cells, which can result, for example, from a mutation in the 5′ flanking region of the PD-1 gene or any other regulatory element of the PD-1 gene, such as a regulatory element located in an intron. Accordingly, an aberrant PD-1 activity can result from an abnormal PD-1 promoter activity, or abnormal enhancer activity.

“Cells,” “host cells” or “recombinant host cells” are terms used interchangeably herein. It is understood that such terms refer not only to the particular subject cell but to the progeny or potential progeny of such a cell.

Because certain modifications may occur in succeeding generations due to either mutation or environmental influences, such progeny may not, in fact, be identical to the parent cell, but are still included within the scope of the term as used herein.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

The term ” a homolog of a nucleic acid” refers to a nucleic acid having a nucleotide sequence having a certain degree of homology with the nucleotide sequence of the nucleic acid or complement thereof. A homolog of a double stranded nucleic acid having SEQ ID N^{o}: x is intended to include nucleic acids having a nucleotide sequence which has a certain degree of homology with SEQ ID N^{o}: x or with the complement thereof. Preferred homologs of nucleic acids are capable of hybridising to the nucleic acid or complement thereof.

The term “interact” as used herein is meant to include detectable interactions between molecules, such as can be detected using, for example, a hybridisation assay. The term interact is also meant to include “binding” interactions between molecules. Interactions may be, for example, protein-protein, protein-nucleic acid, protein-small molecule or small molecule-nucleic acid in nature.

The term “intron”, “intronic sequence” or “intronic nucleotide sequence” refers to the nucleotide sequence of an intron or portion thereof.

The term “isolated” as used herein with respect to nucleic acids, such as DNA or RNA, refers to molecules separated from other DNAs or RNAs, respectively, that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

The term “locus” refers to a specific position in a chromosome. For example, a locus of a PD-1 gene refers to the chromosomal position of the PD-1 gene.

The term “modulation” as used herein refers to both upregulation, (i.e., activation or stimulation), for example by agonizing; and downregulation (i.e. inhibition or suppression), for example by antagonizing of a bioactivity (for example expression of a gene).

The term “molecular structure” of a gene or a portion thereof refers to the structure as defined by the nucleotide content (including deletions, substitutions, additions of one or more nucleotides), the nucleotide sequence, the state of methylation, and/or any other modification of the gene or portion thereof.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the terms “adenosine”, “cytidine”, “guanosine”, and thymidine” are used. It is understood that if the nucleic acid is RNA, a nucleotide having a uracil base is uridine.

The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID N^{o}: x refers to the complementary strand of the strand having SEQ ID N^{o}: x or to any nucleic acid having the nucleotide sequence of the complementary strand of SEQ ID N^{o}: x. When referring to a single stranded nucleic acid having the nucleotide sequence SEQ ID N^{o}: x, the complement of this nucleic acid is a nucleic acid having a nucleotide sequence which is complementary to that of SEQ ID N^{o}: x. The nucleotide sequences and complementary sequences thereof are always given in the 5′ to 3′ direction. The term “complement” and “reverse complement” are used interchangeably herein.

A “non-human animal” of the invention can include mammals such as rodents, non-human primates, sheep, goats, horses, dogs, cows, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which an exogenous sequence is found, or in which an exogenous sequence is expressed in some but not all cells of the animal. The term “tissue-specific chimeric animal” indicates that an exogenous sequence is present and/or expressed or disrupted in some tissues, but not others.

The term “operably linked” is intended to mean that the promoter is associated with the nucleic acid in such a manner as to facilitate transcription of the nucleic acid from the promoter.

The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

The terms “protein”, “polypeptide” and “peptide” are used interchangeably herein when referring to a gene product.

The term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

The term “probe” refers to a molecule which can be used to identify the presence of a PD-1 nucleic acid sequence or protein. Such probes may themselves be nucleic acid sequences of PD-1, expression products of PD-1, or binding molecules to PD-1 such as a ligand or antibody.

A “regulatory element”, also termed herein “regulatory sequence is intended to include elements which are capable of modulating transcription from a basic promoter and include elements such as enhancers and silencers. The term “enhancer”, also referred to herein as “enhancer element”, is intended to include regulatory elements capable of increasing, stimulating, or enhancing transcription from a basic promoter. The term “silencer”, also referred to herein as “silencer element” is intended to include regulatory elements capable of decreasing, inhibiting, or repressing transcription from a basic promoter. Regulatory elements are typically present in 5′ flanking regions of genes. However, regulatory elements have also been shown to be present in other regions of a gene, in particular in introns. Thus, it is possible that PD-1 genes have regulatory elements located in introns, exons, coding regions, and 3′ flanking sequences. Such regulatory elements are also intended to be encompassed by the present invention and can be identified by any of the assays that can be used to identify regulatory elements in 5′ flanking regions of genes.

The term “regulatory element” further encompasses “tissue specific” regulatory elements, i.e., regulatory elements which effect expression of the selected DNA sequence preferentially in specific cells (for example, cells of a specific tissue). Gene expression occurs preferentially in a specific cell if expression in this cell type is significantly higher than expression in other cell types. The term “regulatory element” also encompasses non-tissue specific regulatory elements, i.e., regulatory elements which are active in most cell types. Furthermore, a regulatory element can be a constitutive regulatory element, i.e., a regulatory element which constitutively regulates transcription, as opposed to a regulatory element which is inducible, i.e., a regulatory element which is active primarily in response to a stimulus. A stimulus can be, for example, a molecule, such as a hormone, cytokine, heavy metal, phorbol ester, cyclic AMP (cAMP), or retinoic acid.

Regulatory elements are typically bound by proteins, for example, transcription factors. The term “transcription factor” is intended to include proteins or modified forms thereof, which interact preferentially with specific nucleic acid sequences, i.e., regulatory elements, and which in appropriate conditions stimulate or repress transcription. Some transcription factors are active when they are in the form of a monomer. Alternatively, other transcription factors are active in the form of a dimer consisting of two identical proteins or different proteins (heterodimer). Modified forms of transcription factors are intended to refer to transcription factors having a postranslational modification, such as the attachment of a phosphate group. The activity of a transcription factor is frequently modulated by a postranslational modification. For example, certain transcription factors are active only if they are phosphorylated on specific residues. Alternatively, transcription factors can be active in the absence of phosphorylated residues and become inactivated by phosphorylation. A list of known transcription factors and their DNA binding site can be found, for example, in public databases, for example, TFMATRIX Transcription Factor Binding Site Profile database.

As used herein, the term “specifically hybridises” or “specifically detects” refers to the ability of a nucleic acid molecule of the invention to hybridise to at least approximately 6, 12, 20, 30, 40, 50, 60, 70, 80, 90, 100, 110, 120, 130 or 140 consecutive nucleotides of either strand of a PD-1 gene.

The term “substantially pure” or “purified” does not require absolute purity; rather it is intended as a relative definition of purification of starting materials or natural materials to at least one order of magnitude, preferably two or three orders, and more preferably four or five orders of magnitude is expressly contemplated.

PD-1 refers to a programmed cell death protein-1.

The term “PD-1 therapeutic” refers to various forms of PD-1 polypeptides, as well as peptidomimetics, nucleic acids, or small molecules, which can modulate at least one activity of a PD-1 for the treatment of autoimmune disorders such as multiple sclerosis, Type 1 diabetes, rheumatoid arthritis, Sjögrens syndrome, atopy, allergy, systemic lupus erythematosus and diseases associated with SLE which include fatigue, fever, loss of appetite, nausea, weight loss, hives, loss of scalp hair, red “butterfly rash” and raised rash, sensitivity to sun, ulcers in mouth, nose, or vagina, arthritis, joint pain, loss of blood supply to bone, pain, infections within joints, decrease in kidney function including, blood, aberrant amounts of protein or white blood cells in urine, intracerebral haemorrhage, headaches, loss of coordination, memory loss, seizures stokes, anaemia, low white or platelet count, pericardial effusion, heart attack, inflammation and infection in the heart or the lining of the heart, heart valve problems, shortness of breath, cough, inflammation of the lungs and the lining of the lungs, abdominal distress, diarrhoea, enlargement of the liver, loss of appetite, nausea and vomiting, blindness, visual impairment, dryness of the eyes and dryness of the mouth. As used herein, the term “transfection” means the introduction of a nucleic acid, for example, an expression vector, into a recipient cell by nucleic acid-mediated gene transfer. The term “transduction” is generally used herein when the transfection with a nucleic acid is by viral delivery of the nucleic acid. “Transformation”, as used herein, refers to a process in which a cell's genotype is changed as a result of the cellular uptake of exogenous DNA or RNA, and, for example, the transformed cell expresses a recombinant form of a polypeptide or, in the case of anti-sense expression from the transferred gene, the expression of a naturally-occurring form of the recombinant protein is disrupted.

As used herein, the term “transgene” refers to a nucleic acid sequence which has been introduced into a cell. Daughter cells deriving from a cell in which a transgene has been introduced are also said to contain the transgene (unless it has been deleted). A transgene can encode, for example, a polypeptide, or an antisense transcript, partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (for example, it is inserted at a location which differs from that of the natural gene or its insertion results in a knockout). Alternatively, a transgene can also be present in an episome. A transgene can include one or more transcriptional regulatory sequence and any other nucleic acid, (for example intron), that may be necessary for optimal expression of a selected nucleic acid.

A “transgenic animal” refers to any animal, preferably a non-human animal, for example a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of a protein, for example either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

The term transgenic may also refer to a nucleic acid sequence which has been introduced into a plant cell.

The term “treating” as used herein is intended to encompass curing as well as ameliorating at least one symptom of the condition or disease.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

As described below, one aspect of the invention pertains to isolated nucleic acids comprising a polymorphic sequence of a PD-1 gene. This gene being characterised by containing intron and exon nucleic acid sequences encoding PD-1 as well as 5′ and 3′ nucleic acid sequence which flanks the intron and exon nucleic acid sequences. In a preferred embodiment, the invention provides an intronic sequence of the genomic DNA sequence encoding a PD-1 protein, comprising an intronic sequence shown in FIG. 1 or set forth in any of SEQ ID N^{o}s 1 to 5 (and associated sequences) or complements thereof or homologs thereof. Nucleic acids of the invention can function as probes or primers, for example, in methods for determining the identity of an allelic variant of a PD-1 polymorphic region. The nucleic acids of the invention can also be used to determine whether a subject is at risk of developing a disease associated with a specific allelic variant of a PD-1 polymorphic region, for example, a disease or disorder associated with an aberrant PD-1 activity. The nucleic acids of the invention can further be used to prepare PD-1 polypeptides encoded by specific alleles, such as mutant alleles. Such nucleic acids and polypeptides can be used in gene therapy. Polypeptides encoded by specific PD-1 alleles, such as mutant PD-1 polypeptides, can also be used for preparing reagents, for example, antibodies, for detecting PD-1 proteins encoded by these alleles. Accordingly, such reagents can be used to detect mutant PD-1 proteins, for the diagnosis and treatment of autoimmune diseases disorders and conditions. Preferred polymorphs of PD-1 have at least 90% sequence identity any one of the peptides encoded in SEQ ID N^{o}s 35 to 38, or fragments thereof, as shown in FIG. 21.

Certain nucleic acids of the invention comprise an intronic sequence of a PD-1 gene. The term “PD-1 intronic sequence” refers to a nucleotide sequence of an intron of a PD-1 gene. An intronic sequence can be directly adjacent to an exon or located further away from the exons. Preferred nucleic acids of the invention include an intronic sequence of a PD-1 gene which is adjacent to an exon and comprises at least about 3 consecutive nucleotides, at least about 6 consecutive nucleotides, at least about 9 consecutive nucleotides, at least about 12 consecutive nucleotides, at least about 15 consecutive nucleotides, at least about 18 consecutive nucleotides, or at least about 20 consecutive nucleotides. Isolated nucleic acids which comprise a PD-1 intronic sequence which is immediately adjacent to an exon and comprises at least about 25 consecutive nucleotides, at least about 30 consecutive nucleotides, at least about 35 consecutive nucleotides, at least about 40 consecutive nucleotides, at least about 50 consecutive nucleotides, or at least about 100 consecutive nucleotides are also within the scope of the invention. Preferred isolated nucleic acids of the invention also include those having a PD-1 intronic sequence having a nucleotide sequence of at least about 10 nucleotides, at least about 15 nucleotides, at least about 20 nucleotides, at least about 25 nucleotides, at least about 30 nucleotides, at least about 35 nucleotides, at least about 40 nucleotides, at least about 50 nucleotides or at least about 100 nucleotides. Other preferred nucleic acids of the invention can comprise a PD-1 intronic sequence having less than about 10 nucleotides, provided that the nucleotide sequence is novel. Yet other preferred isolated nucleic acids of the invention include PD-1 intronic nucleic acid sequences of a PD-1 intron, having at least about 150 consecutive nucleotides, at least about 200 consecutive nucleotides, at least about 250 consecutive nucleotides, at least about 300 consecutive nucleotides, at least about 350 consecutive nucleotides, at least about 400 consecutive nucleotides, at least about 500 consecutive nucleotides or at least about 1000 consecutive nucleotides.

Preferred nucleic acids of the invention comprise a PD-1 intronic or non codon encoding nucleic acid sequence having a nucleotide sequence shown in FIG. 1, and/or in any of SEQ ID N^{o}s 1, 2, 3, 4, 5, 6 or 7, complement thereof, reverse complement thereof or homolog thereof. In a preferred embodiment, the invention provides an isolated nucleic acid comprising an PD-1 intronic or non codon encoding nucleic acid sequence which is at least about 80% or preferably at least about 98%, and most preferably at least about 99% identical to an intronic nucleotide sequence shown in FIG. 1 or set forth in any of SEQ ID N^{o}s 1, 2, 3, 4, 5, 6 or 7 or a complement thereof. Certain nucleic acids of the invention comprise an exon coding sequence of a PD-1 gene. The term “PD-1 exon of exonic sequence” refers to a nucleotide sequence of an exon of a PD-1 gene. An exonic sequence can be directly adjacent to an intron or located further away from the intron. Preferred nucleic acids of the invention include an exonic sequence of a PD-1 gene which is adjacent to an intron and comprises at least about 3 consecutive nucleotides, at least about 6 consecutive nucleotides, at least about 9 consecutive nucleotides, at least about 12 consecutive nucleotides, at least about 15 consecutive nucleotides, at least about 18 consecutive nucleotides, or at least about 20 consecutive nucleotides. Isolated nucleic acids 10 which comprise a PD-1 exonic sequence which is immediately adjacent to an intron and comprises at least about 25 consecutive nucleotides, at least about 30 consecutive nucleotides, at least about 35 consecutive nucleotides, at least about 40 consecutive nucleotides, at least about 50 consecutive nucleotides, or at least about 100 consecutive nucleotides are also within the scope of the invention. Preferred isolated nucleic acids of the invention also include those having a PD-1 exonic sequence having a nucleotide sequence of at least about 10 nucleotides, at least about 15 nucleotides, at least about 20 nucleotides, at least about 25 nucleotides, at least about 30 nucleotides, at least about 35 nucleotides, at least about 40 nucleotides, at least about 50 nucleotides or at least about 100 nucleotides. Other preferred nucleic acids of the invention can comprise a PD-1 exonic sequence having less than about 10 nucleotides, provided that the nucleotide sequence is novel. Yet other preferred isolated nucleic acids of the invention include PD-1 exonic nucleic acid sequences of a PD-1 exon, having at least about 150 consecutive nucleotides, at least about 200 consecutive nucleotides, at least about 250 consecutive nucleotides, at least about 300 consecutive nucleotides, at least about 350 consecutive nucleotides, at least about 400 consecutive nucleotides, at least about 500 consecutive nucleotides or at least about 1000 consecutive nucleotides.

Preferred nucleic acids of the invention comprise a PD-1 exonic or codon encoding nucleic acid sequence having a nucleotide sequence shown in FIG. 1, and/or in any of SEQ ID N^{o}s 1, 8, 9, 10, 11 or 12, or complement thereof, reverse complement thereof or homolog thereof. In a preferred embodiment, the invention provides an isolated nucleic acid comprising an PD-1 exonic or codon encoding nucleic acid sequence which is at least about 80% or preferably at least about 98%, and most preferably at least about 99% identical to an intronic nucleotide sequence shown in FIG. 1 or set forth in any of SEQ ID N^{o}s 1, 8, 9, 10, 11 or 12, or a complement thereof. In fact, as described herein, several alleles of human PD-1 genes have been identified. The invention is intended to encompass all of these alleles and PD-1 alleles not yet identified, which can be identified, for example, according to the methods described herein.

Preferred nucleic acids of the invention are from vertebrate genes encoding PD-1 proteins. Particularly preferred vertebrate nucleic acids are mammalian nucleic acids. A particularly preferred nucleic acid of the invention is a human nucleic acid, such as a nucleic acid comprising an PD-1 intronic or non codon encoding nucleic acid sequence shown in FIG. 1 or set forth in any of SEQ ID N^{o}s 1, 2, 3, 4, 5, 6 or 7, Other preferred nucleic acid sequences are those which encode exonic sequences in humans shown in FIG. 1 or set forth in any SEQ ID N^{o}s 1, 8, 9, 10, 11 or 12.

Another aspect of the invention provides a nucleic acid which hybridises under appropriate stringency to an PD-1 intronic or non codon coding nucleic acid sequences having a nucleotide sequence shown in introns shown in FIG. 1 or in intronic sequences set forth in any of SEQ ID N^{o}s 1, 2, 3, 4, 5, 6 or 7 or complement thereof. A further aspect of the invention provides a nucleic acid which hybridises under appropriate stringency to an PD-1 exonic or codon coding nucleic acid sequences having a nucleotide sequence shown in FIG. 1 or in exonic sequences set forth in any of SEQ ID N^{o}s 1, 8, 9, 10, 11 or 12, or complement thereof. Appropriate stringency conditions which promote DNA hybridisation, for example, 6.0.times. sodium chloride/sodium citrate (SSC) at about 45.degree. C., followed by a wash of 2.0.times.SSC at 50.degree. C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0.times.SSC at 50.degree. C. to a high stringency of about 0.2.times.SSC at 50.degree. C. In addition, the temperature in the wash step can be increased from low stringency conditions at room temperature, about 22.degree. C., to high stringency conditions at about 65.degree. C. Both temperature and salt may be varied, or temperature or salt concentration may be held constant while the other variable is changed. In a preferred embodiment, a nucleic acid of the present invention will bind to at least about 20, preferably at least about 25, more preferably at least about 30 and most preferably at least about 50 consecutive nucleotides of a sequence shown in FIG. 1 or set forth in any of SEQ ID N^{o} 1, 2, 3, 4, 5, 6 or 7 under moderately stringent conditions, for example at about 2.0.times.SSC and about 40.degree. C. Even more preferred nucleic acids of the invention are capable of hybridising under stringent conditions to an intronic sequence of at least about 20, 30, 40, or at least about 50 nucleotides as shown in FIG. 1 or as set forth in an intronic or non codon encoding nucleic acid sequence of any of SEQ ID N^{o}s 1, 2, 3, 4, 5, 6 or 7.

Hybridisation, as described above, can be used to isolate nucleic acids comprising an PD-1 intron or portion thereof from various animal species. A comparison of these nucleic acids should be indicative of intronic sequences which may have a regulatory or other function, since these regions are expected 25 to be conserved among various species. Hybridisation can also be used to isolate PD-1 alleles.

The nucleic acid of the invention can be single stranded DNA (for example, an oligonucleotide), double stranded DNA (for example, double stranded oligonucleotide) or RNA. Preferred nucleic acids of the invention can be used as probes or primers. Primers of the invention refer to nucleic acids which hybridise to a nucleic acid sequence which is adjacent to the region of interest or which covers the region of interest and is extended. A primer can be used alone in a detection method, or a primer can be used together with at least one other primer or probe in a detection method. Primers can also be used to amplify at least a portion of a nucleic acid. Probes of the invention refer to nucleic acids which hybridise to the region of interest and which are not further extended. For example, a probe is a nucleic acid which hybridises to a polymorphic region of a PD-1 gene, and which by hybridisation or absence of hybridisation to the DNA of a subject will be indicative of the identity of the allelic variant of the polymorphic region of the PD-1 gene.

Numerous procedures for determining the nucleotide sequence of a nucleic acid, or for determining the presence of mutations in nucleic acids include a nucleic acid amplification step, which can be carried out by, for example, polymerase chain reaction (PCR). Accordingly, in one embodiment, the invention provides primers for amplifying portions of a PD-1 gene, such as portions of exons and/or portions of introns. In a preferred embodiment, the exons and/or sequences adjacent to the exons of the human PD-1 gene will be amplified to, for example, detect which allelic variant of a polymorphic region is present in the PD-1 gene of a subject. Preferred primers comprise a nucleotide sequence complementary to an PD-1 intronic sequence or a specific allelic variant of an PD-1 polymorphic region and of sufficient length to selectively hybridise with an PD-1 gene. In a preferred embodiment, the primer, for example, a substantially purified oligonucleotide, comprises a region having a nucleotide sequence which hybridises under stringent conditions to consecutive nucleotides of an PD-1 gene. In an even more preferred embodiment, the primer is capable of hybridising to a PD-1 intron or non codon encoding sequence and has a nucleotide sequence of an intronic or non codon encoding sequence shown in FIG. 1 or set forth in any of SEQ ID N^{o}s 1 to 7 complements thereof, allelic variants thereof, or complements of allelic variants thereof. For example, primers comprising a nucleotide sequence of at least about 10 consecutive nucleotides, at least about 20 nucleotides or having from about 15 to about 25 nucleotides or set forth in any of SEQ ID N^{o}s 13 to 34 or complement thereof are provided by the invention. Primers having a sequence of more than about 25 nucleotides are also within the scope of the invention. Preferred primers of the invention are primers that can be used in PCR for amplifying each of the exons of a PD-1 gene. Even more preferred primers of the invention have the nucleotide sequence set forth in any of SEQ ID N^{o}s 13 to 34.

Primers can be complementary to nucleotide sequences located close to each other or further apart, depending on the use of the amplified DNA. For example, primers may be chosen such that they amplify DNA fragments of at least about 10 nucleotides or as much as several kilobases. Preferably, the primers of the invention will hybridise selectively to nucleotide sequences located about 50 to about 9000 nucleotides apart.

For amplifying at least a portion of a nucleic acid, a forward primer (i.e., 5′ primer) and a reverse primer (i.e., 3′ primer) will preferably be used. Forward and reverse primers hybridise to complementary strands of a double stranded nucleic acid, such that upon extension from each primer, a double stranded nucleic acid is amplified. A forward primer can be a primer having a nucleotide sequence or a portion of the nucleotide sequence shown in FIG. 1 or in any one of SEQ ID N^{o}s 1, 2, 3, 4, 5, 6, 7, 8, 9, 10, 11 or 12. A reverse primer can be a primer having a nucleotide sequence or a portion of the nucleotide sequence that is complementary to a nucleotide sequence shown in FIG. 1 or in any one of SEQ ID N^{o}s 1, 2, 3, 4, 5, 6, 7, 8, 9, 10, 11 or 12. Preferred forward primers comprise a nucleotide sequence set forth in SEQ ID N^{o}s. 13 to 34. Preferred reverse primers comprise a nucleotide sequence set forth in SEQ ID N^{o}s 13 to 34.

Yet other preferred primers of the invention are nucleic acids which are capable of selectively hybridising to an allelic variant of a polymorphic region of a PD-1 gene. Thus, such primers can be specific for a PD-1 gene sequence, so long as they have a nucleotide sequence which is capable of hybridising to a PD-1 gene. Preferred primers are capable of specifically hybridising to an allelic variant indicated in FIG. 1 and positions 126, 6371, 7101, 7809, 7872, 8162, 8288, 8448 or 9400 respectively. Such primers can be used, for example, in sequence specific oligonucleotide priming as described further herein.

The PD-1 nucleic acids of the invention may also be used as probes, for example, in therapeutic and diagnostic assays. For instance, the present invention provides a probe comprising a substantially purified oligonucleotide, which oligonucleotide comprises a region having a nucleotide sequence that hybridises under stringent conditions to at least approximately 6, 8, 10 or 12, preferably about 25, 30, 40, 50 or 75 consecutive nucleotides of an PD-1 gene. In one embodiment, the probes preferably hybridise to an intron of an PD-1 gene, having an intronic or non codon encoding nucleotide sequence shown in FIG. 1 or set forth in any of SEQ ID N^{o}s 1, 2, 3, 4, 5, 6 or 7 allelic variants thereof, complements thereof or complements of allelic variants thereof. In another embodiment, the probes are capable of hybridising to a nucleotide sequence encompassing an intron/exon border of a PD-1 gene, or complements thereof. In a further embodiment the probes are capable of hybridising to a nucleic acid sequence encoding a PD-1 amino acid sequence, such as those shown for example in FIG. 1, and SEQ ID N^{o}s 1, 8, 9, 10, 11 or 12.

Other preferred probes of the invention are capable of hybridising specifically to a region of a PD-1 gene which is polymorphic. In an even more preferred embodiment of the invention, the probes are capable of hybridising specifically to one allelic variant of an PD-1 gene. Such probes can then be used to specifically detect which allelic variant of a polymorphic region of a PD-1 gene is present in a subject. The polymorphic region can be located in the promoter, exon, intron or 3′UTR sequences of a PD-1 gene.

In preferred embodiments, the probe or primer further comprises a label attached thereto, which, for example, is capable of being detected, for example the label group is selected from amongst radioisotopes, fluorescent compounds, enzymes, and enzyme co-factors.

In a preferred embodiment of the invention, the isolated nucleic acid, which is used, for example, as a probe or a primer, is modified, such as to become more stable. Exemplary nucleic acid molecules which are modified include phosphoramidate, phosphothioate and methylphosphonate analogs of DNA (see also U.S. Pat. Nos. 5,176,996; 5,264,564; and 5,256,775).

The nucleic acids of the invention can also be modified at the base moiety, sugar moiety, or phosphate backbone, for example, to improve stability of the molecule. The nucleic acids, for example, probes or primers, may include other appended groups such as peptides (for example, for targeting host cell receptors in vivo), or agents facilitating transport across the cell membrane (see, for example, Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988), triggered-triggered cleavage agents. (See, for example, Krol et al., 1988, Bio Techniques 6:958-976) or intercalating agents. (See, for example, Zon, 1988, Pharm. Res. 5:539-549). To this end, the nucleic acid of the invention may be conjugated to another molecule, for example, a peptide, hybridisation triggered cross-linking agent, transport agent, triggered-triggered cleavage agent, and the like.

The isolated nucleic acid comprising an PD-1 intronic sequence may comprise at least one modified base moiety which is selected from the group including but not limited to 5-fluorouracil, 5-bromouracil, 5-chlorouracil, 5-iodouracil, hypoxanthine, xantine, 4-acetylcytidine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytidine, 5-methylcytidine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytidine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The isolated nucleic acid may also comprise at least one modified sugar moiety selected from the group including but not limited to arabinose, 2-fluoroarabinose, xylulose, and hexose.

In yet another embodiment, the nucleic acid comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the nucleic acid is an α-anomeric oligonucleotide. An α-anomeric oligonucleotide forms specific double-stranded hybrids with complementary RNA in which, contrary to the usual P-units, the strands run parallel to each other (Gautier et al., 1987, Nucl Acids Res. 15:6625-6641). The oligonucleotide is a 2′-0-methylribonucleotide (Inoue et al., 1987, Nucl. Acids Res. 15:6131-6148), or a chimeric RNA-DNA analogue (Inoue et al., 1987, FEBS Lett. 215:327-330).

Any nucleic acid fragment of the invention can be prepared according to methods well known in the art and described, for example, in Sambrook, J. Fritsch, E. F., and Maniatis, T. (1989) Molecular Cloning: A Laboratory Manual, Cold Spring Harbor Laboratory Press, Cold Spring Harbor, N.Y. For example, discrete fragments of the DNA can be prepared and cloned using restriction enzymes. Alternatively, discrete fragments may be prepared using the Polymerase Chain Reaction (PCR) using primers having an appropriate sequence.

Oligonucleotides of the invention may be synthesized by standard methods known in the art, for example by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The invention also provides vectors and plasmids containing the nucleic acids of the invention. For example, in one embodiment, the invention provides a vector comprising at least a portion of a PD-1 gene comprising a polymorphic region and/or intronic sequence. Thus, the invention provides vectors for expressing at least a portion of the newly identified allelic variants of the human PD-1 gene, as well as other allelic variants. The allelic variants can be expressed in eukaryotic cells, for example, cells of a subject, or in prokaryotic cells.

In one embodiment, the vector comprising at least a portion of a PD-1 allele is introduced into a host cell, such that a protein encoded by the allele is synthesized. The PD-1 protein produced can be used, for example, for the production of antibodies, which can be used, for example, in methods for detecting mutant forms of PD-1. Alternatively, the vector can be used for gene therapy, and be, for example, introduced into a subject to produce PD-1 protein. Host cells comprising a vector having at least a portion of a PD-1 gene are also within the scope of the invention.

The present invention makes available isolated PD-1 polypeptides, such as PD-1 polypeptides which are encoded by specific allelic variants of PD-1, such as those identified herein. In one embodiment, the PD-1 polypeptides are isolated from, or otherwise substantially free of other cellular proteins. The term “substantially free of other cellular proteins” (also referred to herein as “contaminating proteins”) or “substantially pure or purified preparations” are defined as encompassing preparations of PD-1 polypeptides having less than about 20% (by dry weight) contaminating protein, and preferably having less than about 5% contaminating protein. Functional forms of the subject polypeptides can be prepared, for the first time, as purified preparations by using a cloned gene as described herein.

Preferred PD-1 proteins of the invention have an amino acid sequence which is at least about 90%, or 95% identical or homologous to an expression product of the nucleic acid of SEQ ID N^{o} 1 or fragments thereof. Even more preferred PD-1 proteins comprise an amino acid sequence which is at least about 97, 98, or 99% homologous or identical to an amino acid sequence of SEQ ID N^{o} 1 or fragments thereof. Preferred polymorphs of PD-1 have at least 90% sequence identity any one of the peptides encoded in SEQ ID N^{o}s 35 to 38, or fragments thereof, as shown in FIG. 21. Such proteins can be recombinant proteins, and can be, for example, produced in vitro from nucleic acids comprising a specific allele of a PD-1 polymorphic region. For example, recombinant polypeptides preferred by the present invention can be encoded by a nucleic acid, which is at least 80% homologous and more preferably 90% homologous and most preferably 95% homologous with a nucleotide sequence set forth in SEQ ID N^{o} 1 or fragment thereof. Polypeptides which are encoded by a nucleic acid that is at least about 98-99% homologous with the sequence of SEQ ID N^{o} 1 and comprises an allele of a polymorphic region that differs from that set forth in SEQ ID N^{o} 1 are also within the scope of the invention.

In a preferred embodiment, a PD-1 protein of the present invention is a mammalian PD-1 protein. In an even more preferred embodiment, the PD-1 protein is a human protein, such as an PD-1 polypeptide comprising any of the polymorphic amino acid sequence encoded by SEQ ID N^{o} 1. In a most preferred embodiment the polymorphs of PD-1 have at least 90% sequence identity any one of the peptides encoded in SEQ ID N^{o}s 35 to 38, or fragments thereof, as shown in FIG. 21, or an expression product o SEQ ID N^{o} 1.

Full length proteins or fragments corresponding to one or more particular motifs and/or domains or to arbitrary sizes, for example, at least 5, 10, 25, 50, 75 and 100, amino acids in length are within the scope of the present invention.

Isolated peptidyl portions of PD-1 proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, an PD-1 polypeptide of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (for example, “authentic”) PD-1 protein.

In general, polypeptides referred to herein as having an activity (for example, are “bioactive”) of an PD-1 protein are defined as polypeptides which mimic or antagonize all or a portion of the biological/biochemical activities of an PD-1 protein having a nucleic acid sequence of SEQ ID N^{o} 1. Other biological activities of the subject PD-1 proteins are described herein or will be reasonably apparent to those skilled in the art. According to the present invention, a polypeptide has biological activity if it is a specific agonist or antagonist of a naturally-occurring form of an PD-1 protein.

Assays for determining whether a PD-1 protein or variant thereof, has one or more biological activities are well known in the art.

Other preferred proteins of the invention are those encoded by the nucleic acids set forth in the section pertaining to nucleic acids of the invention. In particular, the invention provides fusion proteins, for example, PD-1-immunoglobulin fusion proteins. Such fusion proteins can provide, for example, enhanced stability and solubility of PD-1 proteins and may thus be useful in therapy. Fusion proteins can also be used to produce an immunogenic fragment of an PD-1 protein. For example, the VP6 capsid protein of rotavirus can be used as an immunologic carrier protein for portions of the PD-1 polypeptide, either in the monomeric form or in the form of a viral particle. The nucleic acid sequences corresponding to the portion of a subject PD-1 protein to which antibodies are to be raised can be incorporated into a fusion gene construct which includes coding sequences for a late vaccinia virus structural protein to produce a set of recombinant viruses expressing fusion proteins comprising PD-1 epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a PD-1 protein and the poliovirus capsid protein can be created to enhance immunogenicity of the set of polypeptide antigens (see, for example, EP Publication No: 0259149;, and Evans et al. (1989) Nature 339:385; Huang et al. (1988) J. Virol. 62:3855; and Schlienger et al. (1992) J. Virol. 66:2).

The Multiple antigen peptide system for peptide-based immunization can also be utilized to generate an immunogen, wherein a desired portion of a PD-1 polypeptide is obtained directly from organo-chemical synthesis of the peptide onto an oligomeric branching lysine core (see, for example, Posnett et al. (1988) JBC 263:1719 and Nardelli et al. (1992) J. Immunol. 148:914). Antigenic determinants of PD-1 proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the PD-1 polypeptides of the present invention. For example, PD-1 polypeptides can be generated as glutathione-S-transferase (GST-fusion) proteins. Such GST-fusion proteins can enable easy purification of the PD-1 polypeptide, as for example by the use of glutathione-derivatized matrices (see, for example, Current Protocols in Molecular Biology, eds. Ausubel et al. (N.Y.: John Wiley & Sons, 1991).

The present invention further pertains to methods of producing the subject PD-1 polypeptides. For example, a host cell transfected with a nucleic acid vector directing expression of a nucleotide sequence encoding the subject polypeptides can be cultured under appropriate conditions to allow expression of the peptide to occur. Suitable media for cell culture are well known in the art. The recombinant PD-1 polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant PD-1 polypeptide is a fusion protein containing a domain which facilitates its purification, such as GST fusion protein.

Moreover, it will be generally appreciated that, under certain circumstances, it may be advantageous to provide homologs of one of the subject PD-1 polypeptides which function in a limited capacity as one of either a PD-1 agonist (mimetic) or a PD-1 antagonist, in order to promote or inhibit only a subset of the biological activities of the naturally-occurring form of the protein. Thus, specific biological effects can be elicited by treatment with a homolog of limited function, and with fewer side effects relative to treatment with agonists or antagonists which are directed to all of the biological activities of naturally occurring forms of PD-1 proteins.

Homologs of each of the subject PD-1 proteins can be generated by mutagenesis, such as by discrete point mutation(s), or by truncation. For instance, mutation can give rise to homologs which retain substantially the same, or merely a subset, of the biological activity of the PD-1 polypeptide from which it was derived. Alternatively, antagonistic forms of the protein can be generated which are able to inhibit the function of the naturally occurring form of the protein, such as by competitively binding to an PD-1 receptor.

The recombinant PD-1 polypeptides of the present invention also include homologs of PD-1 polypeptides which differ from the PD-1 proteins encoded by the nucleic acid of SEQ ID N^{o} 1, such as versions of those protein which are resistant to proteolytic cleavage, as for example, due to mutations which alter ubiquitination or other enzymatic targeting associated with the protein.

PD-1 polypeptides may also be chemically modified to create PD-1 derivatives by forming covalent or aggregate conjugates with other chemical moieties, such as glycosyl groups, lipids, phosphate, acetyl groups and the like. Covalent derivatives of PD-1 proteins can be prepared by linking the chemical moieties to functional groups on amino acid side chains of the protein or at the N-terminus or at the C-terminus of the polypeptide.

Modification of the structure of the subject PD-1 polypeptides can be for such purposes as enhancing therapeutic or prophylactic efficacy, stability (for example, ex vivo shelf life and resistance to proteolytic degradation), or post-translational modifications (for example, to alter phosphorylation pattern of protein). Such modified peptides, when designed to retain at least one activity of the naturally-occurring form of the protein, or to produce specific antagonists thereof, are considered functional equivalents of the PD-1 polypeptides described in more detail herein. Such modified peptides can be produced, for instance, by amino acid substitution, deletion, or addition. The substitutional variant may be a substituted conserved amino acid or a substituted non-conserved amino acid.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 2.sup.nd ed., Ed. by L. Stryer, W H Freeman and Co.: 1981). Whether a change in the amino acid sequence of a peptide results in a functional PD-1 homolog (for example, functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

Kits as set forth herein, the invention provides methods, for example, diagnostic and therapeutic methods, for example, for determining the type of allelic variant of a polymorphic region present in a PD-1 gene, such as a human PD-1 gene. In preferred embodiments, the methods use probes or primers comprising nucleotide sequences which are complementary to a PD-1 intronic sequence or to a polymorphic region of a PD-1 gene. Accordingly, the invention provides kits for performing these methods.

In a preferred embodiment, the invention provides a kit for determining whether a subject has or is at risk of developing a disease or condition associated with a specific allelic variant of a PD-1 polymorphic region. In an even more preferred embodiment, the disease or disorder is characterized by an abnormal PD-1 activity. In an even more preferred embodiment, the invention provides a kit for determining whether a subject has or is at risk of developing a cardiovascular disease, for example, autoimmune disorders such as multiple sclerosis, Type 1 diabetes, rheumatoid arthritis, Sjögrens syndrome, atopy, allergy, systemic lupus erythematosus and diseases associated with SLE. A preferred kit provides reagents for determining whether a male or female subject is likely to develop SLE or is suffering from SLE.

Preferred kits comprise at least one probe or primer which is capable of specifically hybridising to a PD-1 sequence or polymorphic region and instructions for use. The kits preferably comprise at least one of the above described nucleic acids, for example, including nucleic acids hybridising to an exon/intron border. Preferred kits for amplifying at least a portion of a PD-1 gene. Even more preferred kits comprise a pair of primers selected from the group consisting of SEQ ID N^{o}s 13 to 34, or complement thereof.

The kits of the invention can also comprise one or more control nucleic acids or reference nucleic acids, such as nucleic acids comprising a PD-1 intronic sequence. For example, a kit can comprise primers for amplifying a polymorphic region of a PD-1 gene and a control DNA corresponding to such an amplified DNA and having the nucleotide sequence of a specific allelic variant. Thus, direct comparison can be performed between the DNA amplified from a subject and the DNA having the nucleotide sequence of a specific allelic variant. In one embodiment, the control nucleic acid comprises at least a portion of a PD-1 gene of an individual, who does not have an autoimmune disease, disorder or condition, associated with an aberrant PD-1 activity.

Yet other kits of the invention comprise at least one reagent necessary to perform the assay. For example, the kit can comprise an enzyme. Alternatively the kit can comprise a buffer or any other necessary reagent.

The invention further features predictive medicines, which are based, at least in part, on determination of the identity of PD-1 polymorphic regions which are associated with specific diseases, conditions or disorders.

For example, information obtained using the diagnostic assays described herein (alone or in conjunction with information on another genetic defect, which contributes to the same disease) is useful for diagnosing or confirming that a symptomatic subject has an allele of a polymorphic region which is associated with a particular disease or disorder. Alternatively, the information (alone or in conjunction with information on another genetic defect, which contributes to the same disease) can be used prognostically for predicting whether a non-symptomatic subject is likely to develop a disease or condition, which is associated with one or more specific alleles of PD-1 polymorphic regions in a subject. Based on the prognostic information, a doctor can recommend a regimen (for example diet or exercise) or therapeutic protocol, useful for preventing or prolonging onset of the particular disease or condition in the individual.

In addition, knowledge of the identity of a particular PD-1 allele in an individual (the PD-1 genetic profile), alone or in conjunction with information on other genetic defects contributing to the same disease (the genetic profile of the particular disease) allows customisation of therapy for a particular disease to the individual's genetic profile. For example, an individual's PD-1 genetic profile or the genetic profile of a disease or condition associated with a specific allele of a PD-1 polymorphic region, can enable a doctor: 1) to more effectively prescribe a drug that will address the molecular basis of the disease or condition; and 2) to better determine the appropriate dosage of a particular drug. For example, the expression level of PD-1 proteins, alone or in conjunction with the expression level of other genes, known to exacerbate to the same disease, can be measured in many patients at various stages of the disease to generate a transcriptional or expression profile of the disease. Expression patterns of individual patients can then be compared to the expression profile of the disease to determine the appropriate drug and dose to administer to the patient.

The ability to target populations expected to show the highest clinical benefit, based on the PD-1 or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labelling (for example since the use of PD-1 as a marker is useful for optimising effective dose).

The present methods provide means for determining if a subject has (diagnostic) or is at risk of developing (prognostic) a disease, condition or disorder that is associated a specific PD-1 allele, for example, autoimmune disorders such as multiple sclerosis, Type 1 diabetes, rheumatoid arthritis, Sjögrens syndrome, atopy, allergy, systemic lupus erythematosus and diseases associated with SLE. The present invention provides methods for determining the molecular structure of a PD-1 gene, such as a human PD-1 gene, or a portion thereof. In one embodiment, determining the molecular structure of at least a portion of a PD-1 gene comprises determining the identity of the allelic variant of at least one polymorphic region of an PD-1 gene. A polymorphic region of a PD-1 gene can be located in an exon, an intron, at an intron/exon border, or in the promoter of the PD-1 gene, or the 3′UTR.

The invention provides methods for determining whether a subject has, or is at risk of developing, a disease or condition associated with a specific allelic variant of a polymorphic region of a PD-1 gene. Such diseases can be associated with an aberrant PD-1 activity, for example, aberrant PD-1 protein level. An aberrant PD-1 protein level can result from an aberrant transcription or post-transcriptional regulation. Thus, allelic differences in specific regions of a PD-1 gene can result in differences of PD-1 protein due to differences in regulation of expression. In particular, some of the identified polymorphisms in the human PD-1 gene may be associated with differences in the level of transcription, RNA maturation, splicing, or translation of the PD-1 gene or transcription product.

Analysis of one or more PD-1 polymorphic region in a subject can be useful for predicting whether a subject has or is likely to develop a autoimmune disorders such as multiple sclerosis, Type 1 diabetes, rheumatoid arthritis, Sjögrens syndrome, atopy, allergy, systemic lupus erythematosus and diseases associated with SLE.

In preferred embodiments, the methods of the invention can be characterized as comprising detecting, in a sample of cells from the subject, the presence or absence of a specific allelic variant of one or more polymorphic regions of an PD-1 gene. The allelic differences can be: (i) a difference in the identity of at least one nucleotide or (ii) a difference in the number of nucleotides, which difference can be a single nucleotide or several nucleotides. The invention also provides methods for detecting differences in PD-1 genes such as chromosomal rearrangements, for example, chromosomal dislocation. The invention can also be used in prenatal diagnostics.

A preferred detection method is allele specific hybridisation using probes overlapping the polymorphic site and having about 5, 10, 20, 25, or 30 nucleotides around the polymorphic region. Examples of probes for detecting specific allelic variants of the polymorphic regions are probes comprising a nucleotide sequence set forth in any of SEQ ID N^{o}s 13 to 34. In a preferred embodiment of the invention, several probes capable of hybridising specifically to allelic variants are attached to a solid phase support, for example, a “chip”. Oligonucleotides can be bound to a solid support by a variety of processes, including lithography. For example a chip can hold up to 250,000 oligonucleotides or more (GeneChip, Affymetrix). Mutation detection analysis using these chips comprising oligonucleotides, also termed “DNA probe arrays” is described for example, in Cronin et al. (1996) Human Mutation 7:244. In one embodiment, a chip comprises all the allelic variants of at least one polymorphic region of a gene. The solid phase support is then contacted with a test nucleic acid and hybridisation to the specific probes is detected. Accordingly, the identity of numerous allelic variants of one or more genes can be identified in a simple hybridisation experiment.

In other detection methods, it is necessary to first amplify at least a portion of a PD-1 gene prior to identifying the allelic variant. Amplification can be performed, for example, by PCR and/or LCR, according to methods known in the art. In one embodiment, genomic DNA of a cell is exposed to two PCR primers and amplification for a number of cycles sufficient to produce the required amount of amplified DNA. In preferred embodiments, the primers are located between about 50 and 9000 base pairs apart.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of an PD-1 gene and detect allelic variants, for example, mutations, by comparing the sequence of the sample sequence with the corresponding wild-type (control) sequence. Exemplary sequencing reactions include those based on techniques developed by Maxam and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Koster; U.S. Pat. No. 5,547,835 and international patent application Publication Number WO 94/21822 entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Koster;. Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, for example, where only one nucleotide is detected, can be carried out.

Yet other sequencing methods are disclosed, for example, in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”. In some cases, the presence of a specific allele of a PD-1 gene in DNA from a subject can be shown by restriction enzyme analysis. For example, a specific nucleotide polymorphism can result in a nucleotide sequence comprising a restriction site which is absent from the nucleotide sequence of another allelic variant. Similarly, the polylmorphism can be determined by analysing the products or restriction digests.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (Myers, et al. (1985) Science 230:1242). In general, the technique of “mismatch cleavage” starts by providing heteroduplexes formed by hybridising a control nucleic acid, which is optionally labelled, for example, RNA or DNA, comprising a nucleotide sequence of a PD-1 allelic variant with a sample nucleic acid, for example, RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with Si nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, Cotton et al (1988) Proc. Natl Acad Sci USA 85:4397; Saleeba et al (1992) Methods Enzymod. 217:286-295. In a preferred embodiment, the control or sample nucleic acid is labelled for detection.

In other embodiments, alterations in electrophoretic mobility is used to identify the type of PD-1 allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analysing the movement of a nucleic acid comprising the polymorphic region in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridisation, selective amplification, or selective primer extension. For example, oligonucleotide probes may be prepared in which the known polymorphic nucleotide is placed centrally (allele-specific probes) and then hybridised to target DNA under conditions which permit hybridisation only if a perfect match is found (Saiki et al. (1986) Nature 324:163); Saiki et al (1989) Proc. Natl Acad. Sca USA 86:6230; and Wallace et al. (1979) Nucl. Acids Res. 6:3543). Such allele specific oligonucleotide hybridisation techniques may be used for the simultaneous detection of several nucleotide changes in different polylmorphic regions of PD-1. For example, oligonucleotides having nucleotide sequences of specific allelic variants are attached to a hybridising membrane and this membrane is then hybridised with labelled sample nucleic acid. Analysis of the hybridisation signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the centre of the molecule (so that amplification depends on differential hybridisation) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol. Cell Probes 6:1).

In another embodiment, identification of the allelic variant is carried out using an oligonucleotide ligation assay (OLA), as described, for example, in U.S. Pat. No. 4,998,617 and in Landegren, U. et al., Science 241:1077-1080 (1988). The OLA protocol uses two oligonucleotides which are designed to be capable of hybridising to abutting sequences of a single strand of a target. One of the oligonucleotides is linked to a separation marker, for example, biotinylated, and the other is detectably labelled. If the precise complementary sequence is found in a target molecule, the oligonucleotides will hybridise such that their termini abut, and create a ligation substrate. Ligation then permits the labelled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect specific allelic variants of a polymorphic region of a PD-1 gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996)Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labelled with different enzyme reporters, alkaline phosphatase or horseradish peroxidase. This system permits the detection of the two alleles using a high throughput format that leads to the production of two different colours.

The invention further provides methods for detecting single nucleotide polymorphisms in a PD-1 gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, for example, in Mundy, C. R. (U.S. Pat. No. 4,656,127). According to the method, a primer complementary to the allelic sequence immediately 3′ to the polymorphic site is permitted to hybridise to a target molecule obtained from a particular animal or human. If the polymorphic site on the target molecule contains a nucleotide that is complementary to the particular exonuclease-resistant nucleotide derivative present, then that derivative will be incorporated onto the end of the hybridised primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labelled dideoxynucleotide derivatives, which, if complementary to the nucleotide of the polymorphic site will become incorporated onto the terminus of the primer.

An alternative method, known as Genetic Bit Analysis or GBA.TM. is described by Goelet, P. et al. (PCT Appln. No. 92/15712). The method of Goelet, P. et al. uses mixtures of labelled terminators and a primer that is complementary to the sequence 3′ to a polymorphic site. The labelled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A. -C., et al., Genomics 8:684-692 (1990); Kuppuswamy, M. N. et al., Proc. Natl. Acad. Sci. (U.S.A.) 88: 1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA.TM. in that they all rely on the incorporation of labelled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A. -C., et al., Amer. J. Hum. Genet. 52:46-59 (1993)).

For determining the identity of the allelic variant of a polymorphic region located in the coding region of a PD-1 gene, yet other methods than those described above can be used. For example, identification of an allelic variant which encodes a mutated PD-1 protein can be performed by using an antibody specifically recognizing the mutant protein in, for example, immunohistochemistry or immunoprecipitation. Antibodies to wild-type PD-1 protein are described, for example, in Acton et al. (1999) Science 271:518 (anti-mouse PD-1 antibody cross-reactive with human PD-1). Other antibodies to wild-type PD-1 or mutated forms of PD-1 proteins can be prepared according to methods known in the art. Binding assays are known in the art and involve, for example, obtaining cells from a subject, and performing binding experiments with a labelled ligand, to determine whether binding to the mutated form of PD-1 differs from binding to the wild-type of the PD-1.

Antibodies directed against wild type or mutant PD-1 polypeptides or allelic variant thereof, which are discussed above, may also be used in disease diagnostics and prognostics. Such diagnostic methods, may be used to detect abnormalities in the level of PD-1 polypeptide expression, or abnormalities in the structure and/or tissue, cellular, or subcellular location of an PD-1 polypeptide. Structural differences may include, for example, differences in the size, electronegativity, or antigenicity of the mutant PD-1 polypeptide relative to the normal PD-1 polypeptide. Protein from the tissue or cell type to be analysed may easily be detected or isolated using techniques which are well known to one of skill in the art, including but not limited to western blot analysis. For a detailed explanation of methods for carrying out Western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.), which is incorporated herein by reference in its entirety.

This can be accomplished, for example, by immunofluorescence techniques employing a fluorescently labelled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of PD-1 polypeptides. In situ detection may be accomplished by removing a histological specimen from a patient, and applying thereto a labelled antibody of the present invention. The antibody (or fragment) is preferably applied by overlaying the labelled antibody (or fragment) onto a biological sample. Through the use of such a procedure, it is possible to determine not only the presence of the PD-1 polypeptide, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod.

Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labelling an anti-PD-1 polypeptide specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labelling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

It is also possible to label the antibody with a fluorescent compound. When the fluorescently labelled antibody is exposed to light of the proper wave length, its presence can then be detected due to fluorescence. Among the most commonly used fluorescent labelling compounds are fluorescein isothiocyanate, rhodamine, phycoerythrin, phycocyanin, allophycocyanin, o-phthaldehyde and fluorescamine.

The antibody can also be detectably labelled using fluorescence emitting metals such as ¹⁵²Eu or others of the lanthanide series. These metals can be attached to the antibody using such metal chelating groups as diethylenetriaminepentacetic acid (DTPA) or ethylenediaminetetraacetic acid (EDTA).

The antibody also can be detectably labelled by coupling it to a chemiluminescent compound. The presence of the chemiluminescent-tagged antibody is then determined by detecting the presence of luminescence that arises during the course of a chemical reaction. Examples of particularly useful chemiluminescent labelling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labelling are luciferin, luciferase and aequorin.

Moreover, it will be understood that any of the above methods for detecting alterations in a gene or gene product or polymorphic variants can be used to monitor the course of treatment or therapy.

If a polymorphic region is located in an exon, either in a coding or non-coding portion of the gene, the identity of the allelic variant can be determined by determining the molecular structure of the MRNA, pre-mRNA, or cDNA. The molecular structure can be determined using any of the above described methods for determining the molecular structure of the genomic DNA, for example, sequencing and SSCP.

The methods described herein may be performed, for example, by utilizing pre-packaged diagnostic kits, such as those described above, comprising at least one probe or primer nucleic acid described herein, which may be conveniently used, for example, to determine whether a subject has or is at risk of developing a disease associated with a specific PD-1 allelic variant.

Sample nucleic acid for using in the above-described diagnostic and prognostic methods can be obtained from any cell type or tissue of a subject. For example, a subject's bodily fluid (for example blood) can be obtained by known techniques (for example venipuncture). Alternatively, nucleic acid tests can be performed on dry samples (for example hair or skin). Fetal nucleic acid samples can be obtained from maternal blood as described in International Patent Application No. WO91/07660 to Bianchi. Alternatively, amniocytes or chorionic villi may be obtained for performing prenatal testing.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridisation: protocols and applications, Raven Press, N.Y.).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

Pharmacogenomics Knowledge of the identity of the allele of one or more PD-1 gene polymorphic regions in an individual (the PD-1 genetic profile), alone or in conjunction with information on other genetic defects contributing to the same disease (the genetic profile of the particular disease) allows a customisation of the therapy for a particular disease to the individual's genetic profile, the goal of “pharmacogenomics”. For example, subjects having a specific allele of a PD-1 gene may or may not exhibit symptoms of a particular disease or be predisposed to developing symptoms of a particular disease. Further, if those subjects are symptomatic, they may or may not respond to a certain drug, for example, a specific PD-1 therapeutic, but may respond to another. Thus, generation of a PD-1 genetic profile, (for example, categorization of alterations in PD-1 genes which are associated with the development of a particular disease), from a population of subjects, who are symptomatic for a disease or condition that is caused by or contributed to by a defective and/or deficient PD-1 gene and/or protein (a PD-1 genetic population profile) and comparison of an individual's PD-1 profile to the population profile, permits the selection or design of drugs that are expected to be safe and efficacious for a particular patient or is patient population (i.e., a group of patients having the same genetic alteration).

For example, an PD-1 population profile can be performed by determining, the PD-1 profile, for example, the identity of PD-1 alleles, in a patient population having a disease, which is associated with one or more specific alleles of PD-1 polymorphic regions. Optionally, the PD-1 population profile can further include information relating to the response of the population to an PD-1 therapeutic, using any of a variety of methods, including, monitoring: 1) the severity of symptoms associated with the PD-1 related disease, 2) PD-1 gene expression level, 3) PD-1 mRNA level, and/or 4) PD-1 protein level. and (iii) dividing or categorizing the population based on particular PD-1 alleles. The PD-1 genetic population profile can also, optionally, indicate those particular PD-1 alleles which are present in patients that are either responsive or non-responsive to a particular therapeutic. This information or population profile, is then useful for predicting which individuals should respond to particular drugs, based on their individual PD-1 profile.

In a preferred embodiment, the PD-1 profile is a transcriptional or expression level profile and step (i) is comprised of determining the expression level of PD-1 proteins, alone or in conjunction with the expression level of other genes known to contribute to the same disease at various stages of the disease.

Pharmacogenomic studies can also be performed using transgenic animals. For example, one can produce transgenic mice, for example, as described herein, which contain a specific allelic variant of a PD-1 gene. These mice can be created, for example, by replacing their wild-type PD-1 gene with an allele of the human PD-1 gene. The response of these mice to specific PD-1 therapeutics can then be determined.

The ability to target populations expected to show the highest clinical benefit, based on the PD-1 or disease genetic profile has been described above.

In situations in which the disease associated with a specific PD-1 allele is characterized by an abnormal PD-1 expression, the treatment of an individual is with a PD-1 therapeutic can be monitored by determining PD-1 characteristics, such as PD-1 protein level or activity, PD-1 mRNA level, and/or PD-1 transcriptional level. This measurement will indicate whether the treatment is effective or whether it should be adjusted or optimised. Thus, PD-1 can be used as a marker for the efficacy of a drug during clinical trials.

In a preferred embodiment, the present invention provides a method for monitoring the effectiveness of treatment of a subject with an agent (for example, an agonist, antagonist, peptidomimetic, protein, peptide, nucleic acid, small molecule, or other drug candidate identified by the screening assays described herein) comprising the steps of (i) obtaining a preadministration sample from a subject prior to administration of the agent; (ii) detecting the level of expression of a PD-1 protein, mRNA, or genomic DNA in the preadministration sample; (iii) obtaining one or more post-administration samples from the subject; (iv) detecting the level of expression or activity of the PD-1 protein, mRNA, or genomic DNA in the post-administration samples; (v) comparing the level of expression or activity of the PD-1 protein, mRNA, or genomic DNA in the preadministration sample with the PD-1 protein, mRNA, or genomic DNA in the post administration sample or samples; and (vi) altering the administration of the agent to the subject accordingly. For example, increased administration of the agent may be desirable to increase the expression or activity of PD-1 to higher levels than detected, i.e., to increase the effectiveness of the agent. Alternatively, decreased administration of the agent may be desirable to decrease expression or activity of PD-1 to lower levels than detected, i.e., to decrease the effectiveness of the agent.

Cells of a subject may also be obtained before and after administration of a PD-1 therapeutic to detect the level of expression of genes other than PD-1, to verify that the PD-1 therapeutic does not increase or decrease the expression of genes which could be deleterious. This can be done, for example, by using the method of transcriptional profiling. Thus, mRNA from cells exposed in vivo to a PD-1 therapeutic and mRNA from the same type of cells that were not exposed to the PD-1 therapeutic could be reverse transcribed and hybridised to a chip containing DNA from numerous genes, to thereby compare the expression of genes in cells treated and not treated with a PD-1 therapeutic. If, for example a PD-1 therapeutic turns on the expression of a proto-oncogene in an individual, use of this particular PD-1 therapeutic may be undesirable.

The present invention provides for both prophylactic and therapeutic methods of treating a subject having or likely to develop a disorder associated with specific PD-1 alleles and/or aberrant PD-1 expression or activity.

In one aspect, the invention provides a method for preventing in a subject, a disease or condition associated with a specific PD-1 allele and/or an aberrant PD-1 expression or activity, by administering to the subject an agent which counteracts the unfavourable biological effect of the specific PD-1 allele. Subjects at risk for such a disease can be identified by a diagnostic or prognostic assay, for example, as described herein. Administration of a prophylactic agent can occur prior to the manifestation of symptoms associated with specific PD-1 alleles, such that a disease or disorder is prevented or, alternatively, delayed in its progression. Depending on the identity of the PD-1 allele in a subject, a compound that counteracts the effect of this allele is administered. The compound can be a compound modulating the level of PD-1 in a patient.

The invention further provides methods of treating subjects having a disease or disorder associated with a specific allelic variant of a polymorphic region of a PD-1 gene. Preferred diseases or disorders include those associated with autoimmune disorders such as multiple sclerosis, Type 1 diabetes, rheumatoid arthritis, Sjögrens syndrome, atopy, allergy, systemic lupus erythematosus and disorders resulting there from. In one embodiment, the method comprises (a) determining the identity of the allelic variant; and (b) administering to the subject a compound that compensates for the effect of the specific allelic variant. The polymorphic region can be localized at any location of the gene, for example, in the promoter (for example, in a regulatory element of the promoter), in an exon, (for example, coding region of an exon), in an intron, or at an exon/intron border. Thus, depending on the site of the polymorphism in the PD-1 gene, a subject having a specific variant of the polymorphic region which is associated with a specific disease or condition, can be treated with compounds which specifically compensate for the allelic variant.

In a preferred embodiment, the identity of one or more of the following nucleotides of a PD-1 gene of a subject is determined: nucleotide 126, 6371, 7101, 7809, 7872, 8162, 8288, 8448 or 9400. Generally, the allelic variant can be a mutant allele, i.e., an allele which when present in one, or preferably two copies, in a subject results in a change in the phenotype of the subject. A mutation can be a substitution, deletion, and/or addition of at least one nucleotide relative to the wild-type allele. Depending on where the mutation is located in the PD-1 gene, the subject can be treated to specifically compensate for the mutation. For example, if the mutation is present in the coding region of the gene and results in an inactive or less active PD-1 protein, the subject can be treated, for example, by administration to the subject of a nucleic acid encoding a wild-type PD-1 protein, such that the expression of the wild-type PD-1 protein compensates for the endogenous mutated form of the PD-1 protein. Nucleic acids encoding wild-type and variant human PD-1 protein are set forth in the expression product of SEQ ID N^{o} 1.

Furthermore, depending on the site of the mutation in the PD-1 protein and the specific effect on its activity, specific treatments can be designed to compensate for that effect. The PD-1 protein is a membrane type protein consisting of 288 amino acids. It contains two hydrophobic regions, one at the N-terminus and the other in the middle, which are likely to serve as a signal peptide and transmembrane segment respectively (U.S. Pat. No. 5,629,204). Thus, if the mutation results in an PD-1 protein which is less capable than the wild type to signal or its level of expression is down regulated, a treatment can be designed which up regulates the expression of PD-1 or improves signal transduction. In one embodiment, a compound or molecule which promotes expression or signal transduction of PD-1, is administered to the subject.

A mutant PD-1 protein can also be an PD-1 protein having a mutation in the cytoplasmic domain of the protein which results in an aberrant signal transduction from the PD-1. Subjects having such a mutation can be treated, for example, by administration of compounds which induce the same or similar signal transduction or compounds which act downstream of PD-1.

The effect of a mutation in a PD-1 protein can be determined according to methods known in the art. For example, if the mutation is located in the extracellular portion of the protein, one can perform binding assays -using an appropriate ligand, and determine whether the binding affinity of such a ligand with the mutated PD-1 protein is different from the binding affinity of the ligand with the wild-type protein. Such assays can be performed using a soluble form of a PD-1 protein or a membrane bound form of the protein. If the mutation in the PD-1 protein is located in the cytoplasmic domain of the protein, signal transduction experiments can be performed to determine whether the signal transduced from the mutated PD-1 differs from the signal transduced from the wild-type PD-1. Alternatively, one can also investigate whether binding to a protein which interacts with the cytoplasmic domain of the receptor is affected by the mutation. Such determination can be made by, for example, by immunoprecipitation.

Yet in another embodiment, the invention provides methods for treating a subject having a mutated PD-1 gene, in which the mutation is located in a regulatory region of the gene. Such a regulatory region can be localized in the promoter of the gene, in the 5′ or 3′ untranslated region of an exon, or in an intron or in the 3′UTR. A mutation in a regulatory region can result in increased production of PD-1 protein, decreased production of PD-1 protein, or production of PD-1 having an aberrant tissue distribution. The effect of a mutation in a regulatory region upon the PD-1 protein can be determined, for example, by measuring the PD-1 protein level or mRNA level in cells having a PD-1 gene having this mutation and which, normally (i.e., in the absence of the mutation) produce PD-1 protein. The effect of a mutation can also be determined in vitro. For example, if the mutation is in the promoter, a reporter construct can be constructed which comprises the mutated promoter linked to a reporter gene, the construct transfected into cells, and comparison of the level of expression of the reporter gene under the control of the mutated promoter and under the control of a wild-type promoter. Such experiments can also be carried out in mice transgenic for the mutated promoter. If the mutation is located in an intron, the effect of the mutation can be determined, for example, by producing transgenic animals in which the mutated PD-1 gene has been introduced and in which the wild-type gene may have been knocked out. Comparison of the, level of expression of PD-1 in the mice transgenic for the mutant human PD-1 gene with mice transgenic for a wild-type human PD-1 gene will reveal whether the mutation results in increased, decreased synthesis of the PD-1 protein and/or aberrant tissue distribution of PD-1 protein. Such analysis could also be performed in cultured cells, in which the human mutant PD-1 gene is introduced and, for example, replaces the endogenous wild-type PD-1 gene in the cell. Thus, depending on the effect of the mutation in a regulatory region of a PD-1 gene, a specific treatment can be administered to a subject having such a mutation. Accordingly, if the mutation results in decreased production of a PD-1 protein, the subject can be treated by administration of a compound which increases synthesis, such as by increasing PD-1 gene expression, and wherein the compound acts at a regulatory element different from the one which is mutated. Alternatively, if the mutation results in increased PD-1 protein levels, the subject can be treated by administration of a compound which reduces PD-1 protein production, for example, by reducing PD-1 gene expression or a compound which inhibits or reduces the activity of PD-1.

A correlation between drug responses and specific alleles of PD-1 can be shown, for example, by clinical studies wherein the response to specific drugs of subjects having different allelic variants of a polymorphic region of a PD-1 gene is compared. Such studies can also be performed using animal models, such as mice having various alleles of human PD-1 genes and in which, for example, the endogenous PD-1 has been inactivated such as by a knock-out mutation. Test drugs are then administered to the mice having different human PD-1 alleles and the response of the different mice to a specific compound is compared. Accordingly, the invention provides assays for identifying the drug which will be best suited for treating a specific disease or condition in a subject. For example, it will be possible to select drugs which will be devoid of toxicity, or have the lowest level of toxicity possible for treating a subject having a disease or condition.

The identification of different alleles of PD-1 can also be useful for identifying an individual among other individuals from the same species. For example, DNA sequences can be used as a fingerprint for detection of different individuals within the same species (Thompson, J. S. and Thompson, eds., Genetics in Medicine, W B Saunders Co., Philadelphia, Pa. (1991)). This is useful, for example, in forensic studies.

Embodiments of the invention will now be described by way of example only, with reference to the accompanying drawings of which the examples that follow are better described with reference to the accompanying drawings of which:—

FIG. 1: Shows the nucleic acid sequence of the human PD-1 gene;

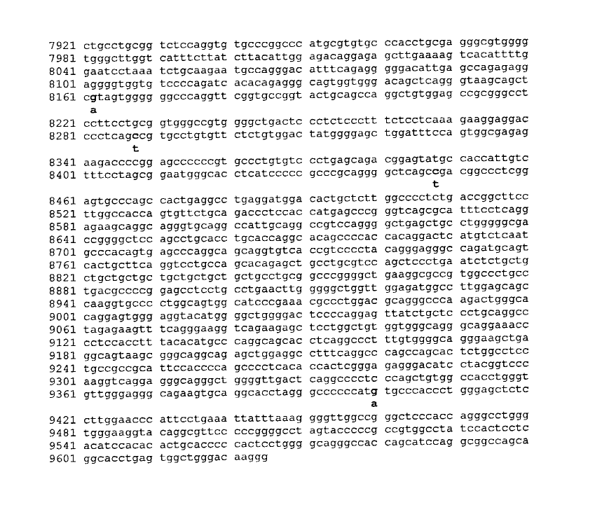

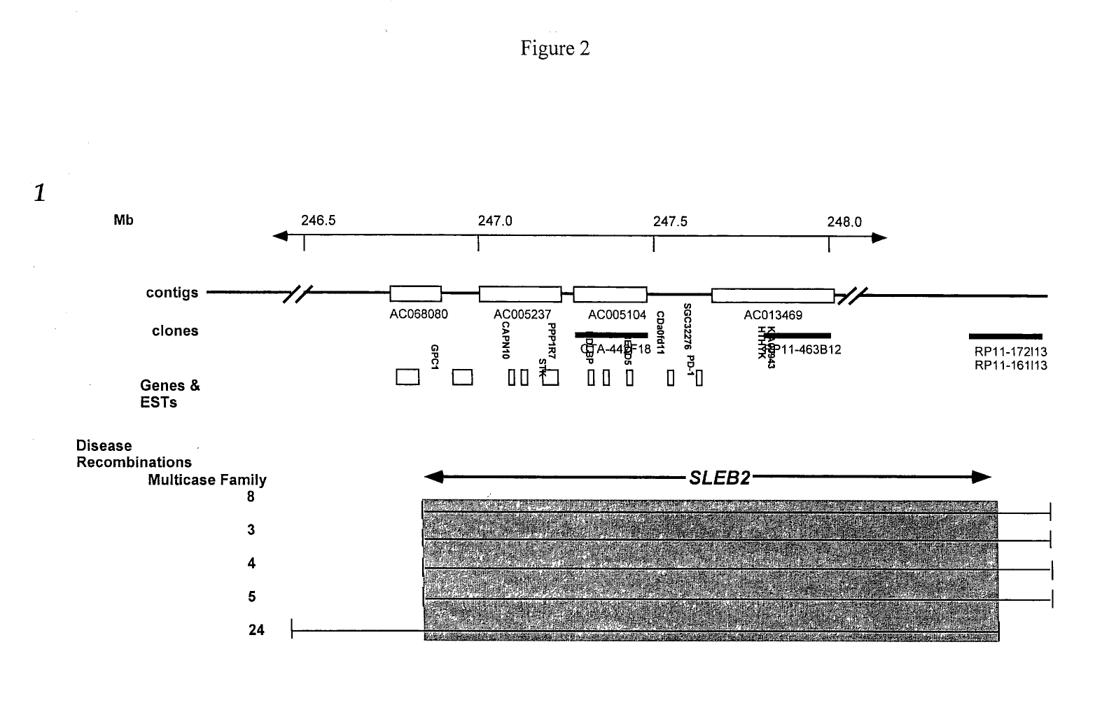

FIG. 2: Shows the physical map of the 2q37.3 region;

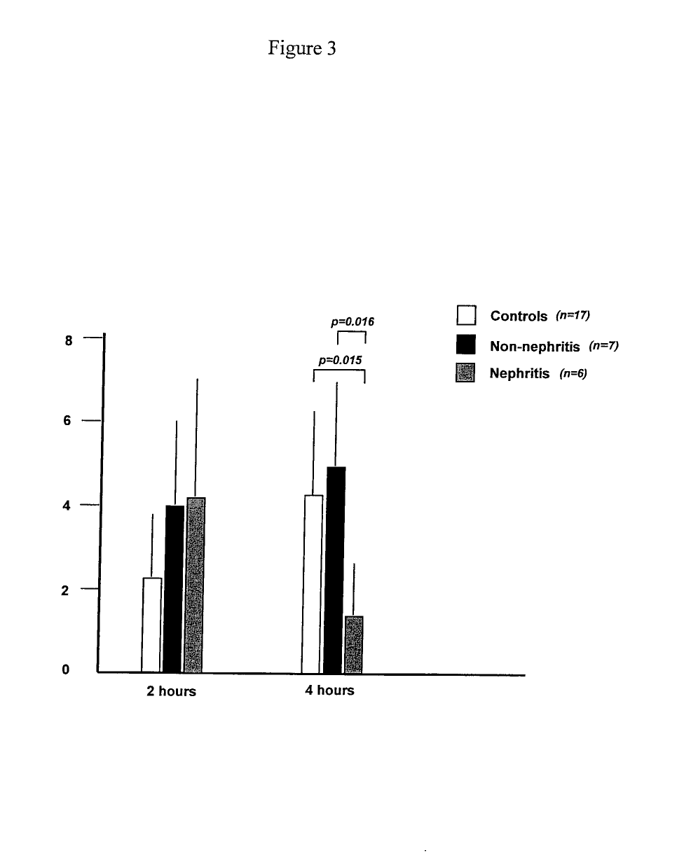

FIG. 3: Shows expression of PD-1 mRNA in patients;

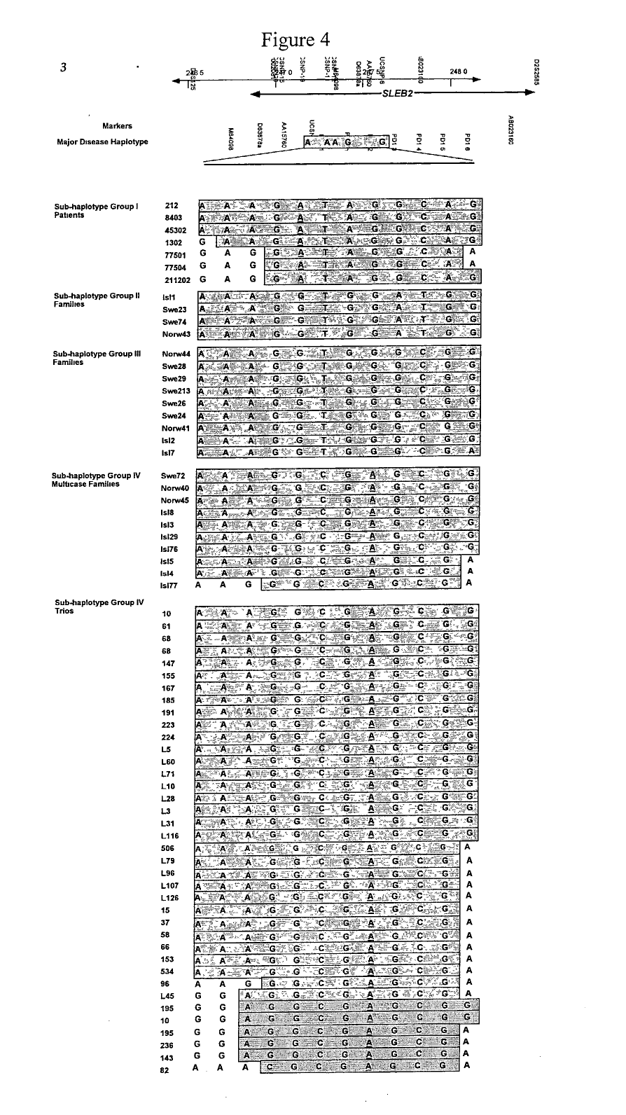

FIG. 4: Shows the major haplotype for the SLEB2 locus and subhaplotype groups identified with the inclusion of the PD-1 polymorphisms;

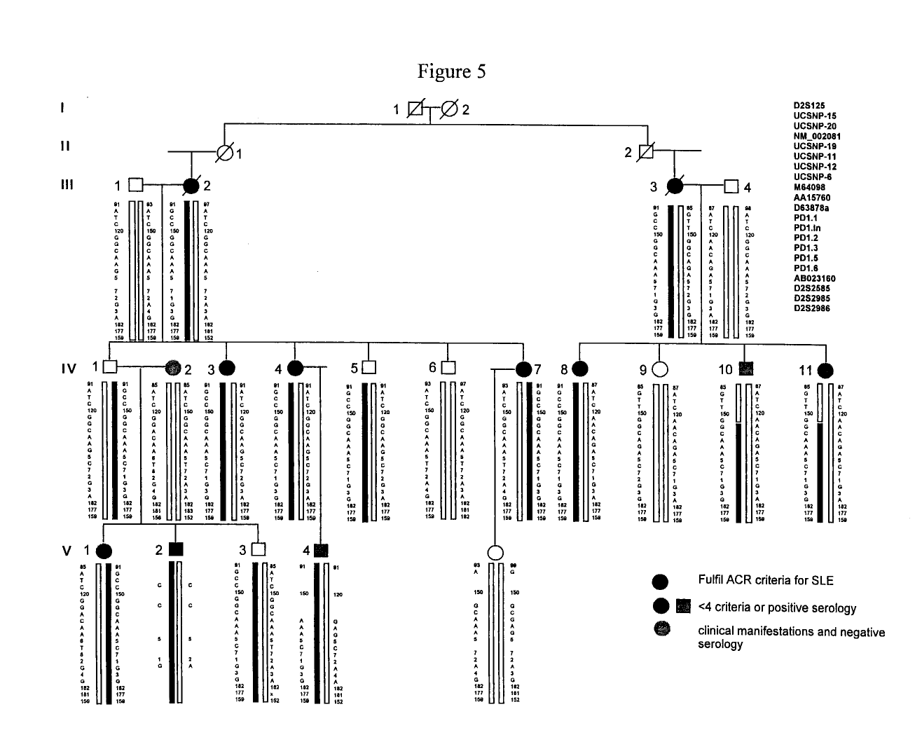

FIG. 5: Shows the geneology of one of the Icelandic families with the group IV disease sub-haplotype containing the PD1.3 A allele and with two affected recombinants (IV-10 and IV-11);

FIG. 6a: Is a map of the 172 bp region of intron 4 of the PD-1 gene containing predicted transcription factor binding sites;

FIG. 6b: Shows the results of an electrophoretic mobility shift assay;

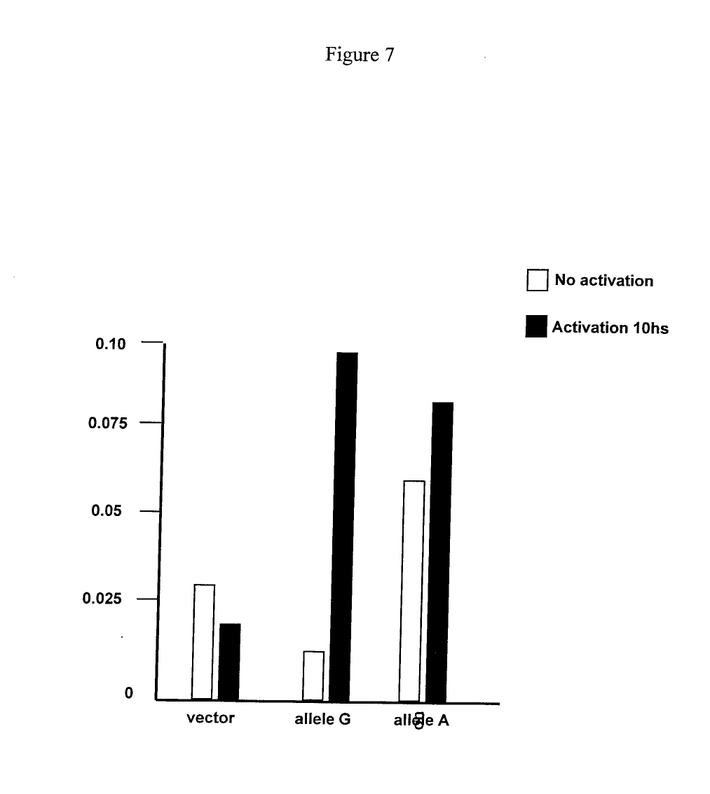

FIG. 7: Shows results of the reporter gene assay for the intron 4 of PD-1;

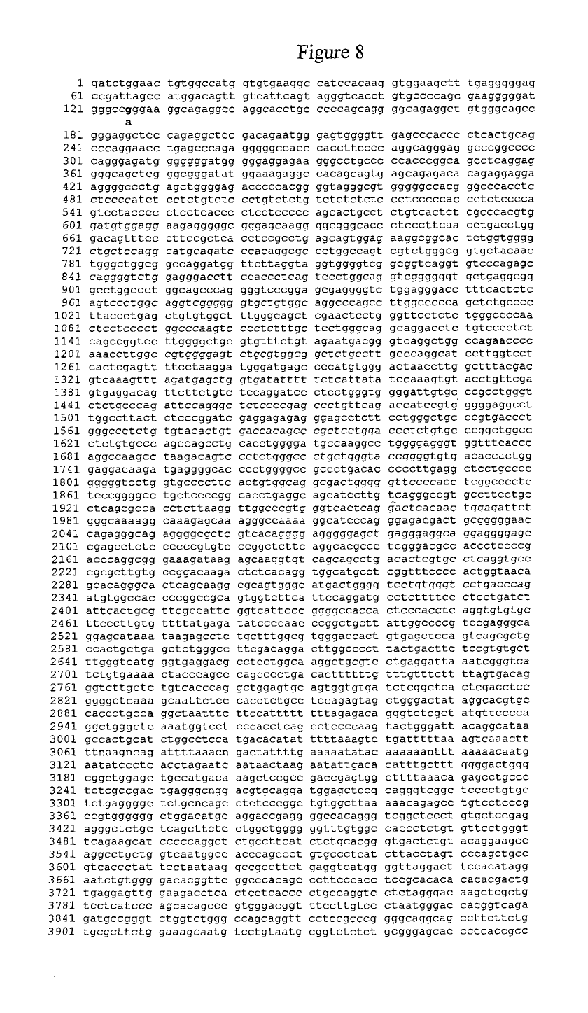

FIG. 8: Shows sequence number 1;

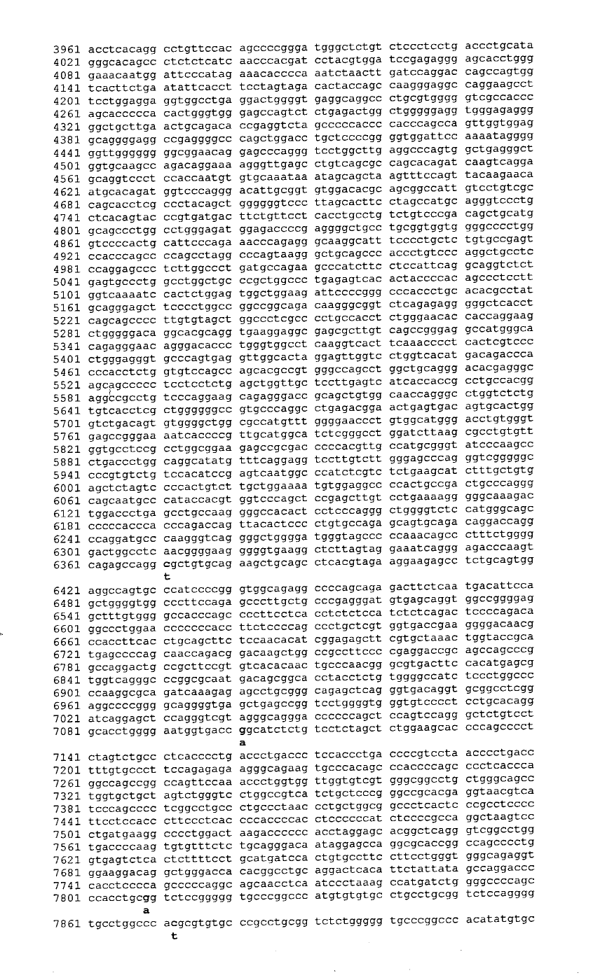

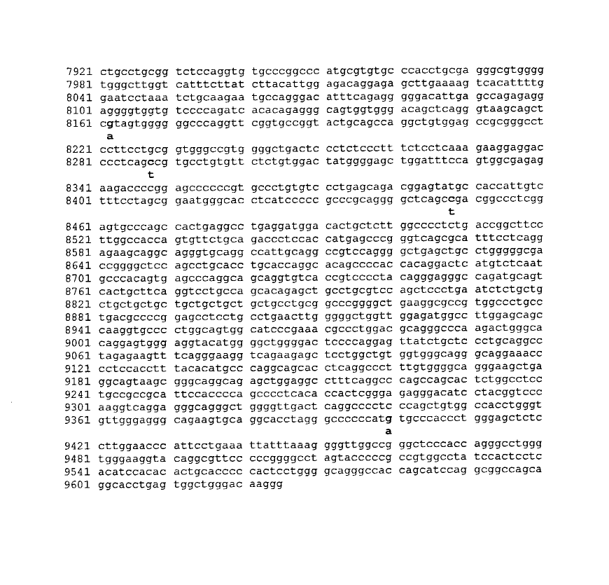

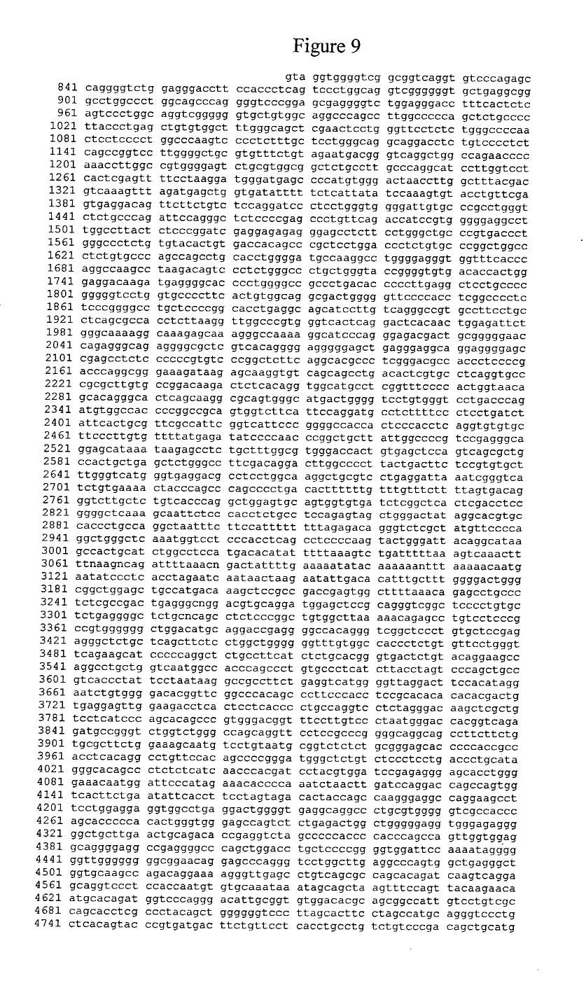

FIG. 9: Shows sequence number 2 which is the nucleic acid sequence encoding intron 1, polymorphism is shown in bold;

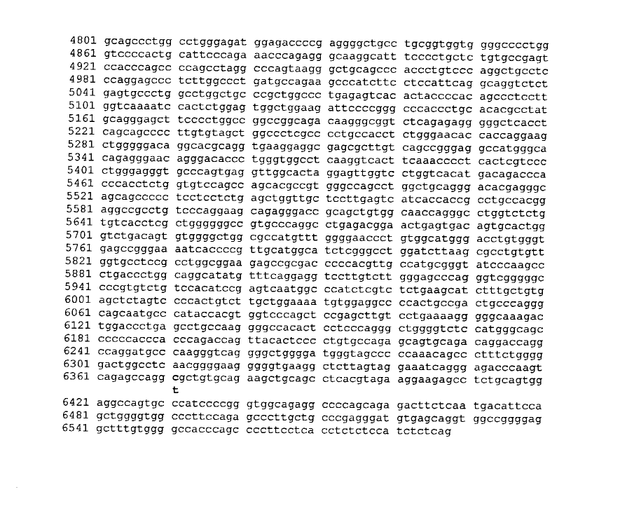

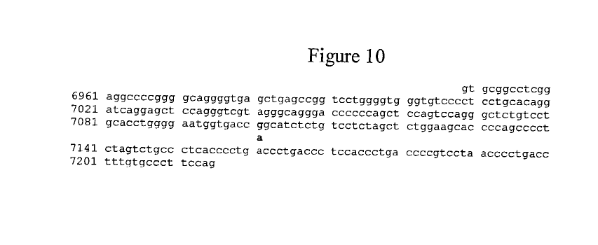

FIG. 10: Shows sequence number 3 which is the nucleic acid sequence encoding intron 2, polymorphism is shown in bold;

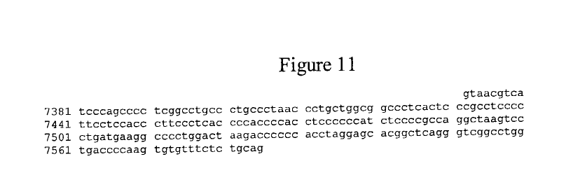

FIG. 11: Shows sequence number 4 which is the nucleic acid sequence encoding intron 3;

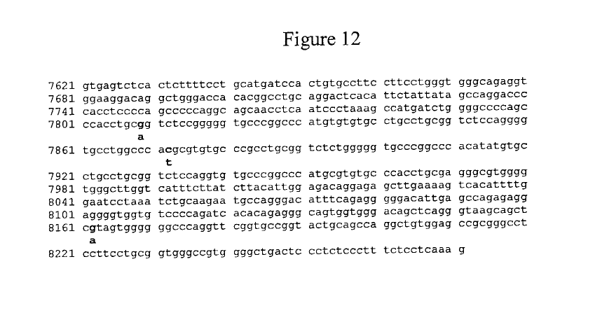

FIG. 12: Shows sequence number 5 which is the nucleic acid sequence encoding intron 4, polymorphism is shown in bold;

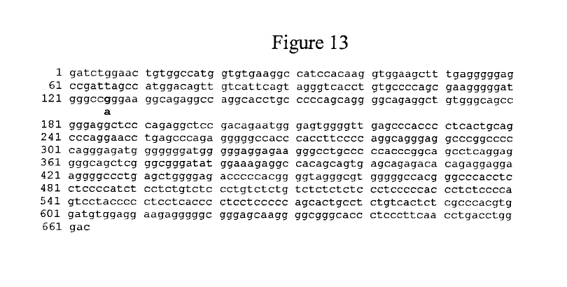

FIG. 13: Shows sequence number 6 which is the nucleic acid sequence encoding Promoter region, polymorphism is shown in bold;

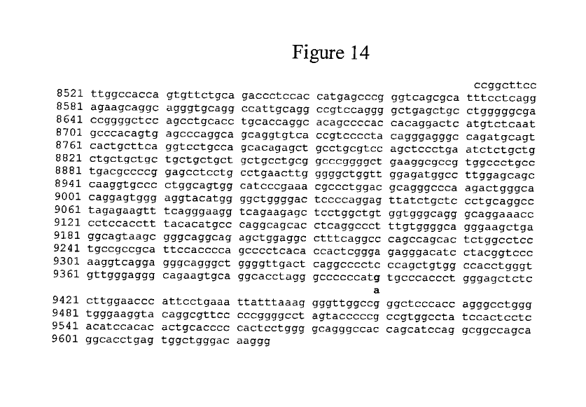

FIG. 14: Shows sequence number 7 which is the nucleic acid sequence encoding 3′UTR, polymorphism us shown in bold;

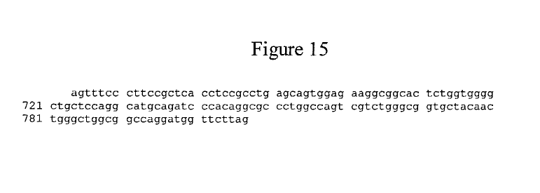

FIG. 15: Shows sequence number 8 which is the nucleic acid sequence encoding exon 1;

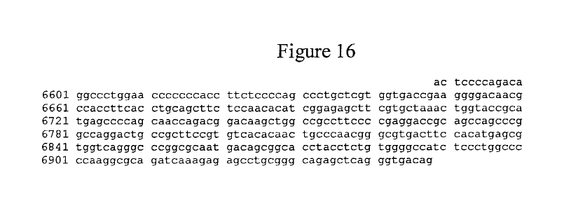

FIG. 16: Shows sequence number 9 which is the nucleic acid sequence encoding exon 2;

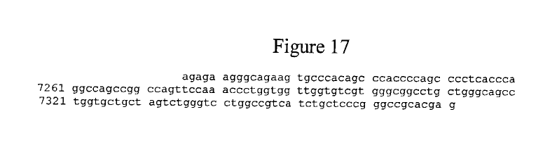

FIG. 17: Shows sequence number 10 which is the nucleic acid sequence encoding exon 3;

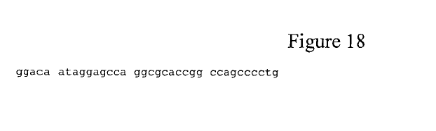

FIG. 18: Shows sequence number 11 which is the nucleic acid sequence encoding the exon 4;

FIG. 19: Shows sequence number 12 which is the nucleic acid sequence encoding exon 5, polymorphism is shown in bold;

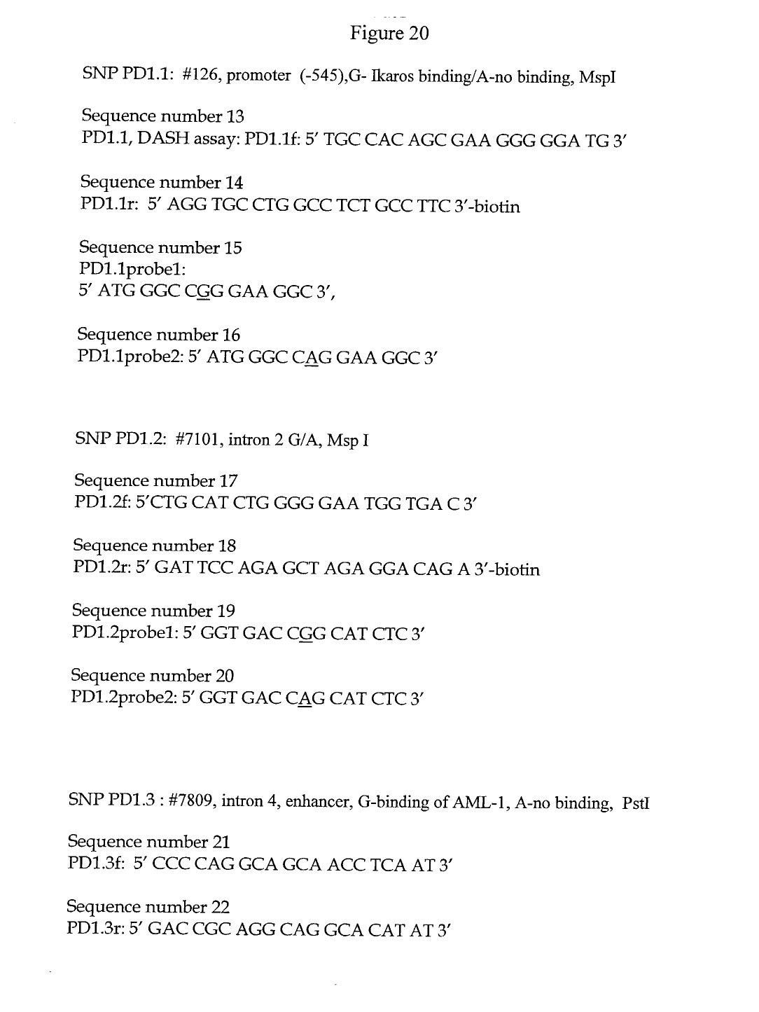

FIG. 20: Shows sequence numbers 13 to 34 which are oligonucleotides;

FIG. 21: Shows sequence numbers 35 to 38 which are polypeptides encoding PD-1.

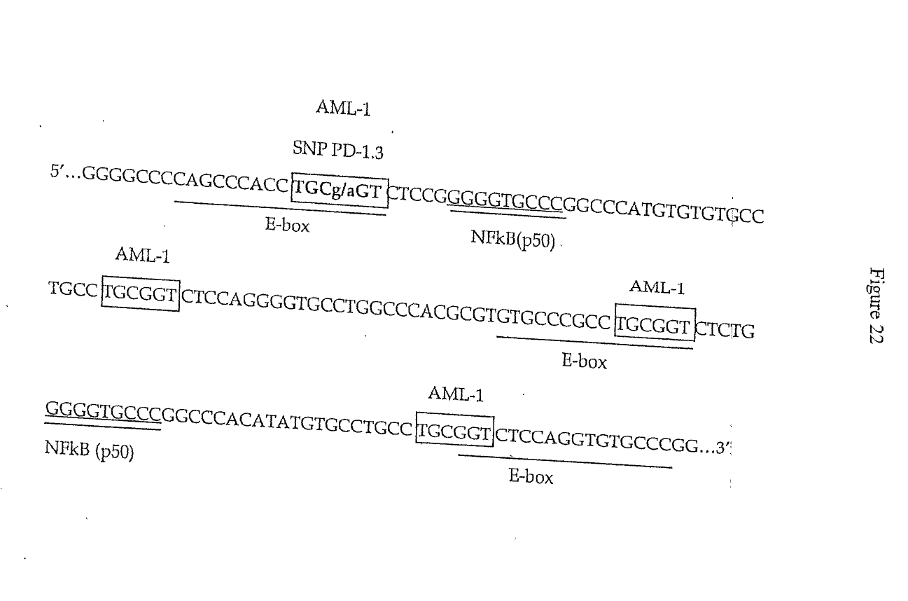

FIG. 22: Shows the structure of the predicted binding sites in the intronic enhancer within the PD-1 gene.

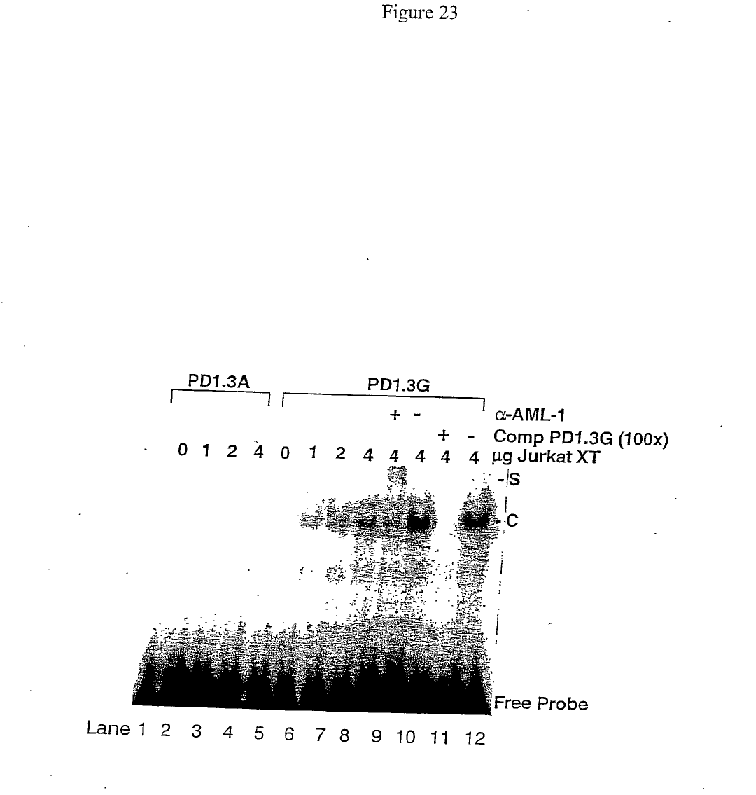

FIG. 23: Shows an electrophoretic mobility shift, supershift and competition assays with Jurkat nuclear cell extract and allelic variants of SNP PD-1.3.

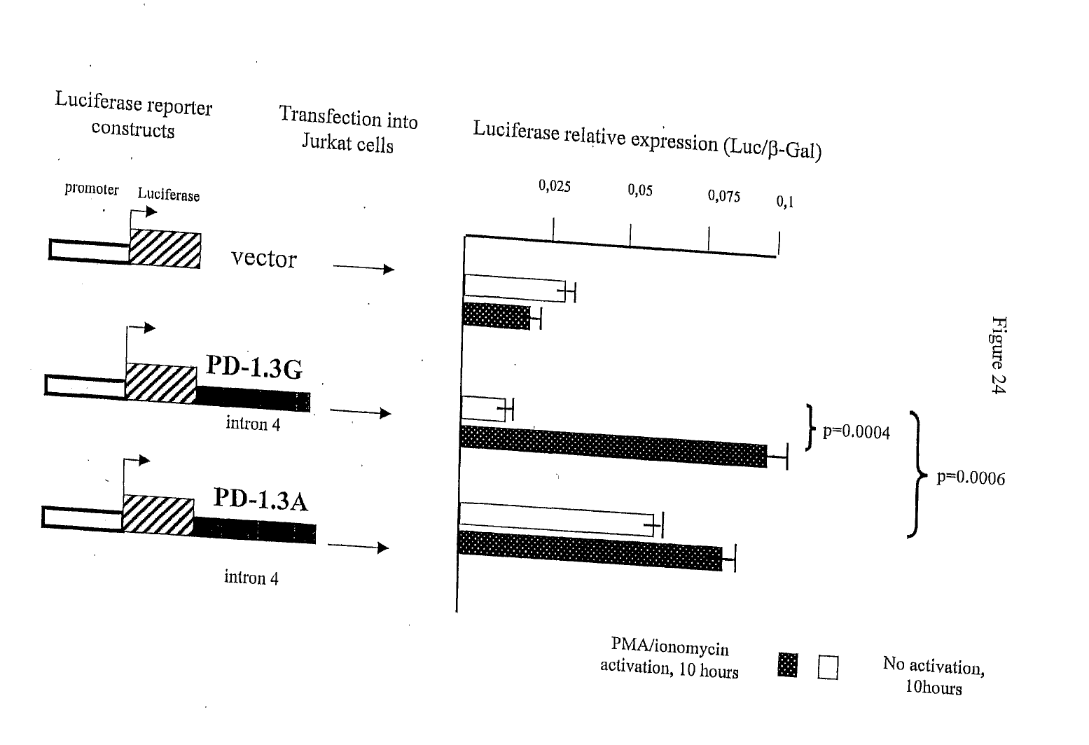

FIG. 24: Shows enhancer activity of intron 4 of the PD-1 gene in a Luciferase reporter assay; and

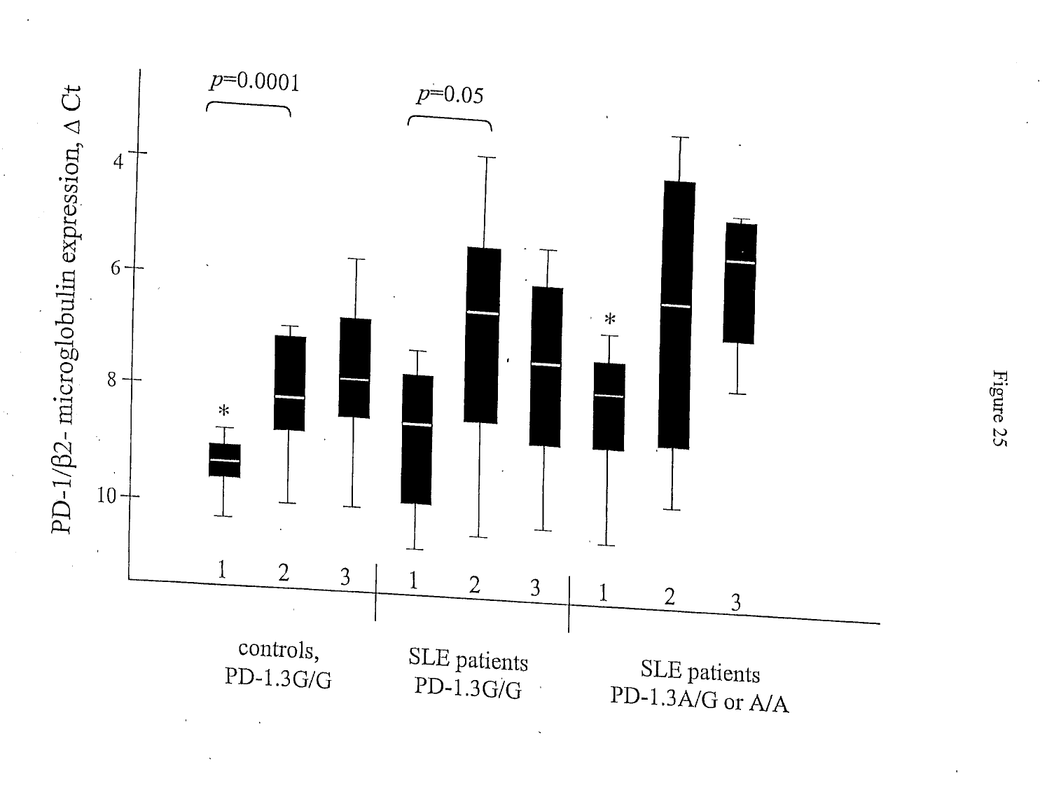

FIG. 25: Shows expression of the PD-1 mRNA in SLE patients and controls.

Referring to FIG. 2, on the map the top line shows the distance in bases, the second line shows contiguous sequences as found in the Ensemble database. The clones used for FISH are shown as black bars. The position of PD-1 and other genes and ESTs relative to the contiguous information available through the public databases is also shown, as well as other genes according to Ensemble and the physical map published for NIDDM1. The extension of the SLEB2 locus is shown as a grey square. The recombinations found in affected individuals and the families to which they belonged are also depicted.

In FIG. 3 the expression of PD-1 mRNA in patients with SLE, with nephritis and without nephritis and healthy controls after stimulation of PBMC with PMA and ionomycin for 2 hours and 4 hours is shown. Mean and standard error are also shown.

In FIG. 4, the major haplotype for the SLEB2 locus and subhaplotype groups are identified with the inclusion of the PD-1 polymorphisms.

The haplotype blocks are shadowed in grey to distinguish the recombinations. The relevant PD-1 allele for the sub-haplotype I and IV is underlined. For sub-haplotype 1, the numbers of the affected individuals with the haplotype are shown. For the other sub-haplotypes, the number of the family is given. In addition this figure shows the recombinational history of the group IV disease sub-haplotype in the multicase families and trios (haplotypes transmitted to the SLE individual) where PD-1.3 A is found. This figure shows how only the polymorphisms of the PD-1 gene are conserved among all individuals having the group IV sub-haplotype.

In FIG. 5 the disease haplotype is shown as a black bar. The alleles of the markers for each haplotype are shown with PD1.3A underlined (shown as a number 1). Individual IV-2 had clinical manifestations but negative serology and is inmarried. However this individual has the sub-haplotype of the group I (including PD1.1) and is related to a second SLE patient not studied here but known to us by genealogy information (not shown).

Turning to FIGS. 6a and 6b, the start and end of the four 40 bp repeats are shown (*). The binding sites are underlined and the transcription factors are shown below the core sequences. The SNP PD1.3 change at the first AML-1 site is in bold. 6b. EMSA demonstrating the lack of binding to the SNP PD1.3 A allele (lanes 1-4) by nuclear extracts of Jurkat cells, and the formation of a DNA-binding complex “C” on the PD1.3G allele (lanes 5-12), specifically competed for by unlabelled PD1.3 G oligonucleotide (lane 11) but not an unspecific oligonucleotide (lane 12). Antisera against AML-1 led to a supershifted band “S” (lane 9), whereas an unrelated antisera did not (lane 10). Electrophoretic mobility shift assay results are illustrated by FIG. 6b.

The bars of FIG. 7 show the Luciferase activity after normalization with the β-gal control. Three independent transfections were performed each in duplicates. A statistical significant difference was observed between allele A and G of PD1.3 in non-activated cells (p=0.001). Jurkat cells were activated with PMA+Ionomycin 8 hours after transfection and Luciferase activity was measured after 10 hours of induction. Activation of the allele G construct increases transcription of the reporter gene by 8,3 fold, while the presence of allele A only results in an increase of transcription by 1.3-fold. Differences were statistically significant (p=0.0004).

### EXAMPLE 1

Recombination Analysis of SLEB2 and Identification of a Major Haplotype Associated With SLE

Linkage studies of multicase families positioned the SLEB2 locus between markers D2S125 and the joined markers D2S2585/D2S2985 with a Iod-3 support interval. We obtained a maximum multipoint Iod score towards the telomere at D2S2585/D2S2985 of Z=6.03 with an “affected-only” analysis¹⁰ and consistent with a dominant mode of inheritance (disease gene frequency of 0.002). The SLEB2 locus overlaps partially with the NIDDM1 locus and a physical map for NIDDM1 (ref. 18) allowed us to position a number of polymorphisms and genes and search for disease-segregating haplotypes.

Four families had affected individuals with recombinations at the centromeric end of the locus that limited the size of SLEB2 from the gene GPC1 to the telomere (246.83-248.0 Mb) (FIG. 2 and FIG. 5 for one of the families). Another family had a recombination at the telomeric end, between the markers D2S2585 (QTEL44) and D2S2986 (QTEL47) (FIG. 2).

Out of 32 haplotypes observed, we identified one within the defined recombinational borders (from GPC1-D2S2985) to be strongly associated with SLE in the multicase families (p<0.00008) and the sporadic cases with parents included (p<0.00001), all of Swedish origin (see Methods). This haplotype is 10 composed of variants of the loci M64098 (HDLBP), D63878a (NEDD5), MA15760 (Cda0fd11), UCSNP-6 (SGC32276) and AB023160 (KIAA0943), A-A-A-G-G, respectively (FIG. 2). These markers were in linkage disequilibrium with each other and all showed some degree of association with SLE in trios (corrected p values ranging from 0.2-0.02). This result supports the hypothesis that this region contains a susceptibility locus for SLE.

### EXAMPLE 2

PD-1 is Located Within SLEB2

Of the few known genes in 2q37.3, only PD-1 was considered a strong candidate for the SLEB2 locus. PD-1 is expressed in lymphocytes and it is known to regulate T and B cell activation and mice made deficient for PD-1 develop a syndrome characterized by high levels of autoantibodies and immune-complex-mediated glomerulonephritis, similar to human SLE¹⁷. This gene had previously been mapped to 2q37.3, but its precise position was not known^{11,12}.

PD-1 was not found among the clones described in the physical map for NIDDM1 (ref. 18) nor in a second unpublished map of the region (provided by Dr. Patrick Concannon as a personal communication) or in any commercially available BAC and PAC libraries. Instead, we used fluorescent in situ hybridisation (FISH) on metaphase and interphase chromosomes to define the position of PD-1 and some other ESTs. This identified the position of PD-1 centromeric of AC025684.00001 (represented by the clone RP11-463B12) at the very end of the 2q37.3 (247.50-247.70 Mb, chr2) (FIG. 2). PD-1 was therefore included within SLEB2. We also positioned the KIAA0943 EST and the HTHYK genes (thymidylate kinase) within this interval and close to PD-1 (between PD-1 and the BAC clone RP11-463B12). We could not unambiguously define the exact position of KIAA0943 and HTHYK in relation to each other (FIG. 2).

### EXAMPLE3

PD-1 is Differentially Expressed in SLE, Nephritis and Controls

The expression of PD-1 in peripheral blood mononuclear cells (PBMC) was studied in 13 female patients, including 6 patients with nephritis and 7 without nephritis and 17 female controls. We stratified patients into these two groups because the PD-1 deficient mice had nephritis as a major manifestation. In order to avoid influence of activity status in gene expression, all patients had an inactive, stable disease at the time of the study and low dose or no treatment (see Methods). PBMCs were activated with a combination of the protein kinase C activator PMA and the Ca²⁺ ionophore ionomycin, having non-treated cells as controls. Cells were harvested at 0, 2 and 4 hours after activation. Expression of PD-1 mRNA was measured by quantitative RT-PCR (TaqMan). The level of PD-1 expression in non-activated samples was 1.9 times higher in SLE patients as compared to controls (p<0.012) (data not shown). In activated samples from controls PD-1 expression was increased at 2 hours and peaked at 4 hours (FIG. 3). SLE patients (with and without nephritis) had higher expression of PD-1 at 2 hours compared with controls (p=0.009). However, the SLE PD-1 expression diverged into two groups at 4 hours: Patients with nephritis differed from both controls (p=0.015) and patients without nephritis (p=0.016) (FIG. 3) in that expression of PD-1 was decreased. We conclude that both the constitutive and induced expression of PD-1 differs between SLE, lupus nephritis and the control group functionally supporting the role of PD-1 in the disease.

### EXAMPLE 4

Polymorphisms of PD-1 Reveal Genetic/Allelic Heterogeneity and the Presence of Founder Disease Haplotypes Within SLEB2

The complete PD-1 gene was sequenced in 10 unrelated individuals from multicase families (4 healthy and 6 with SLE) and 6 single nucleotide polymorphisms were identified in 9,6 kb. PD1.1 located in the promoter, SNP PD1.2 in intron 2, SNP PD1.3 and SNP PD1.4 in intron 4, SNP PD1.5 in exon 5 (synonymous substitution), and SNP PD1.6 in the 3′ UTR. The multicase families were genotyped for the SNPs PD1.3, PD1.5 and PD1.6 and analysed for linkage. PD1.1 and PD1.2 were in complete linkage disequilibirum, as well as PD1.4 and PD1.5. Linkage was detected with the SNPs and SLE (PD1.5, PIC=0.35, Z=3.60, Table 1 A, B and C) and multipoint analysis including the telomeric microsatellite marker D2S2585 increased the Iod score (Z=7.06) (data not shown). This showed linkage of PD-1 to SLE, as expected.

TABLE 1A
Linkage and Association Analysis of PD-1 Polymorphisms in
Scandinavian Multicase Families (n = 31).

| Marker** | Assay | PIC^{§} value | LOD (Z) |
|---|---|---|---|
| PD1.3 (A/G) | RFLP/DASH | 0.22 | 0.96 |
| PD1.5 (T/C) | RFLP | 0.35 | 3.60 |
| PD1.6 (A/G) | RFLP | 0.22 | 0.49 |

The SNPs were assayed by restriction enzyme analysis (RFLP) and/or dynamic allele-specific hybridization (DASH). **The nucleotide change is shown within parentheses. §PIC=polymorphism information content was obtained for the multicase families using DOWNFREQ software by Joseph Terwilliger (Columbia University, New York).

TABLE 1B
Association of PD1.3A to SLE and Lupus Nephritis

| | | | Patients | | |
|---|---|---|---|---|---|
| Controls | | | | | no- |
| Mar- | Population | NTA* | SLE | Nephritis | nephritis |
| ker** | (n = 474)^{§§} | (n = 186) | (n = 508) | (n = 152) | (n = 356) |
| PD1.3 | 0.08/0.92 | 0.04/0.96 | 0.11/0.89 | 0.18/0.82** | 0.08/0.92 |
| (A/G) | | | | | |
| PD1.5 | 0.43/0.57 | ND | 0.40/0.60 | 0.43/0.57 | 0.39/0.61 |
| (T/C) | | | | | |
| PD1.6 | 0.09/0.91 | ND | 0.09/0.91 | 0.09/0.91 | 0.09/0.91 |
| (A/G) | | | | | |

^{§§}Refers to the number of chromosomes.
*NTA refers to the non-transmitted alleles of parents to SLE offspring as a second control group in the analyses.
**X² = 13.2, p = 0.0005 when compared to the population controls, X² = 14, 7, p = 0.0001 when compared with the alleles from parents to SLE patients that were not transmitted to their offspring and X² = 12.9, p = 0.0007 when compared with the patients without nephritis.

TABLE 1C
Evidence for Linkage of the SNPs of PD-1 in Mexican Multicase
Families (n = 30) and Association to PD1.3A in Mexican Sporadic
Patients with Lupus Nephritis

| Linkage | PIC^{§} value | LOD (Z) |
|---|---|---|
| PD1.5 (T/C) | 0.37 | 1.55 |
| Association | SLE patients | Controls |
| | (n = 276)^{§} | (n = 206) |
| PD1.3 (A/G) | 0.12/0.86 | 0.02/0.98* |

*X² = 14.2, p < 0.0001.
^{§}refers to the number of chromosomes.

With the SNPs of PD-1 included in the analysis, the present inventors found that the major haplotype was partitioned and new disease haplotypes were observed supporting the presence of allelic and/or genetic heterogeneity. The present inventors discovered 4 sub-haplotypes segregating with SLE in multicase families (FIG. 4) probably suggesting the presence of at least 4 different underlying mutations. Each sub-haplotype was found in four groups of families I, II, III and IV (FIG. 4); In some cases, two different disease haplotypes were observed in one family (FIG. 4 and individual IV-2 in FIG. 5), suggesting allelic heterogeneity. The sub-haplotype from group I was a unique haplotype associated with a rare variant of the polymorphism PD1.1 in the promoter of PD-1 (data not shown). This haplotype was found only in affected individuals in 7 multicase families and 3 out of 190 sporadic trios, but was not observed in the non-transmitted haplotypes. Sub-haplotypes from groups II and III were observed in both patients and controls. Group II segregated with the disease in four families and group III in 9 families. These haplotypes differed only by the presence of PD1.4 and PD1.5 (in tight LD). None of the alleles for these markers had an obvious or a known association with diseases. These two groups require further investigation and search for other disease-associated polymorphisms within PD-1 or in surrounding genes.

The sub-haplotype from group IV was distinct due to the presence of allele A of PD1.3 that was observed only in one, possibly founder disease haplotype (FIG. 4). This haplotype segregated in 10 out of the 31 multicase families (7 Icelandic, 2 Norwegian and 1 Swedish). All four recombination events delimiting the centromeric end of SLEB2 (FIG. 2 and 5) were found in individuals carrying this haplotype. So we decided to analyse this haplotype and the PD1.3 A variant further. The present inventors studied the association of PD-1 polymorphisms in our independent set of 190 trios. Only PD1.3A was associated with SLE (TDT, p<0.02, transmitted in 11%, non-transmifted in 4%) (Table 1).

Since the inventors did not have more informative recombinants for this sub-haplotype and PD1.3 A was present in a unique disease haplotype, the present inventors analysed all affected individuals with PD1.3 allele A from the multicase families and the trios for the past history of recombinational events. The present inventors reasoned that if PD1.3 A was a mutation occurring only once in a founder haplotype, we could obtain information as to whether PD-1, and in particular PD-1.3A was the susceptibility variant for SLE by studying the decay of the haplotype in past generations (FIG. 4). The closer a marker is to the disease mutation (which PD1.3 A is assumed to be) fewer recombination events will be observed indicating the degree of linkage disequilibrium between the markers in the haplotype and between the markers and the disease mutation. The most conserved unit of the haplotype, segregating with the disease would be the “disease unit”. In 54% of the transmitted haplotypes, PD1.3A was present in a “complete” haplotype covering markers M64098 to AB023160. The telomeric marker AB012360 was excluded in 42% of the haplotypes and markers M64098 and D63878a could be excluded in 13% of the cases. Past recombinations excluding A115760 and UCSNP-6 occurred in 4 and 2% of the cases, respectively. The inventors conclude from this analysis that all but PD-1 markers could be excluded from the “disease unit”. The excluded markers have been recombined out of the disease haplotype sometime in the past with frequencies depending on the degree of linkage disequilibrium in the region and on the degree of polymorphism of each marker. This analysis allows us to exclude other genes beyond PD-1 from consideration in our mutation search (FIG. 4), and give support for PD-1 as the susceptibility gene for SLE in this group of families and for PD1.3A as the susceptibility variant.

### EXAMPLE 5

Association of PD1.3 A With Lupus Nephritis

Glomerulonephritis is an important clinical feature of SLE that usually represents a very defined immune complex induced manifestation and has been observed in mouse models with deficiency of PD-1¹⁷. Our own results showed differences in PD-1 expression between patients with and without nephritis and controls, so the present inventors studied this subgroup of patients for association with the SNPs. Of the 254 Swedish sporadic patients available (including the 190 trios and 64 more sporadic patients), 30% were diagnosed with nephritis (see Methods). The present inventors used as control groups a population set of Swedish ascendance and the non-transmitted alleles (NTA) of the trios (see Methods and Table 1).

Association was found to the PD1.3 A when comparing nephritis patients with both control groups (population controls, X²=13.2, p=0.0005 with Fisher's exact test, corrected p=0.003, RR=2.3 and NTA controls, X²=14,7, p=0.0001, corrected p=0.0006, RR=3.7). Association was also observed when we compared patients with nephritis with those without nephritis (X²=12.9, p=0.0007 corrected p=0.004). No difference was observed between patients without nephritis and any of the control groups (Table 1). Thus, PD1.3A is strongly associated with lupus nephritis in sporadic patients.

The inventors now aimed at defining if PD1.3 could be functionally involved in susceptibility to SLE and nephritis. The inventors found that the SNP PD1.3 lies in intron 4 within a region containing four copies of a 40 bp direct repeat unique for the PD-1 gene. The present inventors observed four predicted sites for the transcription factor AML-1, four E boxes and three sites for NFκB within the repeat (FIG. 6a). The SNP PD1.3 A disrupts the core DNA-binding sequence for AML-1 in the first repeat (FIG. 6a). The present inventors performed an electrophoretic mobility shift assay (EMSA) (FIG. 6b). Nuclear extracts from Jurkat cells expressing AML-1 (Ref. 19) formed a complex with a probe covering the first AML-1 site (PD1.3G in FIG. 6b). In contrast, the probe for PD1.3 A (PD1.3A) failed to form any complex (FIG. 6b). The binding of the extract to the PD1.3G oligonucleotide was specifically competed by unlabelled self-oligonucleotide. Upon addition of AML-1 polyclonal antisera, a supershifted band was detected suggesting that AML-1 indeed is part of a complex that binds to PD1.3G (FIG. 6b, lane 9).

In order to confirm the functional significance of the PD1.3 polymorphism the present inventors cloned the complete intron 4 (560 bp) into the pGL3-promoter vector and transfected the construct into the human T cell line Jurkat. The presence of allele A enhanced the transcriptional activity of the reporter gene, suggesting that a loss of AML-1 binding results in de-repression of basal transcription (p=0.0001 as compared with allele G, FIG. 7). Activation of cells transfected with the construct containing allele G resulted in up to 8-fold increase from the baseline (p=0.0004) while the induction of transcription was only 1.3-fold (p=0.007) in the presence of allele A (FIG. 7). The diminished activation-induced gene transcription by the nephritis-associated allele A is in line with the results obtained for PD-1 expression in the patients with nephritis. These results are consistent with the 40 bp repeat of intron 4 being a regulatory element and that PD1.3 allele A influences its activity.

### EXAMPLE 6

Genetic Evidence for the Role of PD-1 in Susceptibility to Human SLE

As expected for a complex disease, allelic and genetic heterogeneity was found at the SLEB2 locus. It is therefore not possible to assume that a general mutation for the disease could be found. Instead, the present inventors discovered the presence of several (at least 4) independent mutations through haplotype analysis. With such analysis the present inventors could identify conserved fragments of linkage disequilibrium that segregated non-randomly with the disease.

The present inventors found that rare polymorphisms, like PD1.1 and PD1.3 with frequencies between 1-15% could be themselves disease mutations, or alternatively, belong to rare and distinct haplotypes useful for fine mapping. Due to the very rare allelic frequency of PD1.1A (less than 0,5% in controls and about 1% in sporadic SLE and its presence in 7 individuals from the multicase families) no association analysis was applicable at this point. This SNP is, nevertheless, functionally interesting (data not shown). If indeed regulatory, this polymorphism could be relevant for a small proportion of SLE patients. PD1.3 was more polymorphic than PD1.1 and this allowed us to make statistical evaluations, which showed that out of all PD-1 SNPs found, PD1.3 was the strongest candidate as a mutation for PD-1, due to the association of allele A with SLE and lupus nephritis.

Assuming that PD1.3 A was a mutation that had occurred once in a founder disease haplotype, the present inventors analysed its recombinational history. The analysis of past recombinational events allowed us to exclude other markers that, although tightly linked in the major disease haplotype, had undergone recombination from the disease sub-haplotype in past generations. Only PD-1 was conserved. Therefore, PD-1 is the susceptibility gene for the group of families and patients that have PD1.3A, but the present inventors cannot exclude other polymorphisms and/or genes within SLEB2 to be involved in susceptibility to SLE in the other families and patients.

In addition, the present inventors observed that the “affected” recombinants delimiting SLEB2 from the centromeric end were found in families with individuals carrying the PD1.3 A associated haplotype, supporting the exclusion of the region upstream UCSNP-19 from further mutational searching.

Even though the public and private human genome sequence was released recently^{20,21}, the PD-1 gene was not mapped within the “golden path”, or found at any Celera scaffold. The present inventors could not find clones where the PD-1 gene was located. This has not allowed us to further sequence the 2q37.3 region and further study the sub-haplotypes segregating with the other multicase families. In addition 2q37.3 as a telomeric region, is saturated with repetitive elements, a fact that makes cloning difficult. The present inventors have mapped the mouse PD-1 gene to chromosome 1 (unpublished results) and expect to identify other genes in the syntenic region that will help us in characterizing 2q37.3 in more detail.

In conclusion, our gene mapping results and haplotype analysis gave us the necessary evidence for PD-1 as the only candidate gene in the SLEB2 region for the group of families and patients carrying the PD1.3 A-associated haplotype.

### EXAMPLE 7

Functional Evidence for the Role of PD-1 and PD1.3A in Human SLE

The inventors considered PD-1 as a candidate gene from the functional point of view, first, because it was a gene involved in peripheral immune tolerance and inhibition of T cell activation¹²⁻¹⁷. A mouse model made deficient for PD-1 develops, at late age, a lupus-like syndrome with high levels of autoantibodies and glomerulonephritis¹⁷. The second reason was that our expression results suggested differences of PD-1 between patients and controls, and even more, between patients with and without nephritis. The present inventors were careful in choosing patients with inactive and stable disease, in order to avoid SLE activity relapses to influence the results. Despite this, sample variability was observed, but the tendency was clear.

The present inventors observed that the PD1.3 A disrupts binding of an important transcription factor, de-represses basal transcription and is unable to enhance gene transcription in response to cell activation, suggesting a defect on the on-off switch affecting PD-1 expression. This result is in line with the function of PD-1 as an immunoreceptor tyrosine-based inhibitory motif-containing molecule¹¹⁻¹⁷. It should be expected that a decrease in the expression of PD-1 would prevent its inhibitory function upon cellular activation. In general our results support the possibility that the reduced expression of PD-1 after cellular activation or antigenic stimulation in lupus nephritis can be partly explained by the presence of the PD-1.3 allele A.

SLE is a disease characterized by a state of chronic lymphocyte hyperactivity²²⁻²⁴. Persistent T cell activation is thought to allow autoreactive T cells to induce autoantibody production and increase the formation of immune complexes that could eventually deposit in the kidney glomerulus. Genes coding for molecules similar to PD-1, carrying inhibitory motifs (ITIM) have been described as involved in autoimmune-like syndromes when deleted by homologous recombination²⁵ or found as mutations in lupus-prone mice²⁶. These models show T or B cell hyperactivity and autoantibody production. These receptors and many of the pathways in which they act, are involved in the fine regulation of T and B cell activation and tolerance, possibly at different developmental stages. It is tempting to hypothesize that these different genes may be involved in susceptibility to human SLE as well, representing a general mechanism for disease pathogenesis with unique mutations in different populations.

PD-1 contributes to the risk for the expression of the disease possibly together with other factors known to affect immune complex deposition such as partial deficiency of C4A (ref. 4) or reduced handling of immune complexes by the FcG receptors IIA and IIIA^{27, 28}. The present inventors are at present analysing the genetic effect of PD-1 together with other genes associated with SLE.

The polymorphism PD1.3 is located within an intronic direct repeat with transcription factor-binding sites for molecules exclusively involved in hematopoietic differentiation and inflammation²⁹⁻³¹. Previous studies have identified other intronic regulatory sequences^{18,32,33}. Some examples are the silencers for the genes CD4 and CD21, both of which are members of the immunoglobulin superfamily^{32,33} and the recently described polymorphism in intron 3 of calpain 10 (CAPN10) for NIDDM1 (Ref. 18). The present inventors expect to identify other important intronic or non-coding regulatory sequences to play a role in complex disease pathogenesis in the future.

The present inventors show here for the first time the possible role of the transcription factor AML-1 in peripheral tolerance and inflammation. Support for the role of AML-1 in T cell regulation is found in the co-activation by AML-1 on the T cell receptor, alpha chain enhancer³⁴. The present work gives evidence for a regulatory role of AML-1 in T-lymphocyte-expressed co-receptors. The transcription interactions taking place at the regulatory element of the intron 4 of PD-1 appear to be very complex. Within the region, transcription factor binding sites for NFkB and E boxes were also found, but their role in the regulatory element has to be established.

The importance of using extended multicase families from historically and ethnically related and relatively homogeneous populations as the Scandinavian is underscored by the work presented here. The degree of genetic and allelic heterogeneity that was found at the SLEB2 locus should make one reconsider the usefulness of highly admixed populations and affected sib-pair analysis in favour of an approach where pedigree structure and parent information is maximally used for finding genes for complex diseases.

### EXAMPLE 8

Association of PD-1 with Human Disease Using Multicase Multiethnicity Study

The present inventors analyzed here 2510 individuals from 5 independent sets for SNPs found in the PD-1 gene. The present inventors have shown that an intronic SNP in the PD-1 gene is associated with development of SLE in Europeans (p=0.00001, RR=2.6) and Mexicans (p=0.0009, RR=3.5). This SNP alters a binding site for the transcription factor AML-1 in an intronic enhancer. This change causes alterations in the enhancer activity upon cellular activation and in PD-1 mRNA expression in SLE patients.

The PD-1 gene considered a strong candidate for SLEB2 because it is an immunoreceptor, member of the immunoglobulin family, has a tyrosine-based inhibitory motif (ITIM), is known to regulate T and B cell activation and mice knockout for pd-1 develop an SLE-like disease^{11, 12, 14, 17}. Using FISH the present inventors confirmed that the PD-1 gene is uniquely present in the region 2q37.3 (data not shown). Present inventors then sequenced the complete gene in 5 unrelated patients and 5 controls from the Nordic multicase families where SLEB2 was detected and identified 7 SNPs (Table 2).

TABLE 2
Position and Features of SNPs Found in PD-1 Gene

| | Position in | | |
|---|---|---|---|
| | the gene bp | | |
| | from the | | |
| | translation | | |
| SNP | start | Feature | Comments |
| PD-1.1 A/G | −531 | promoter | Frequency <1% in Europeans |
| PD-1.2 A/G | 6438 | intron 2 | Frequency <1% in Europeans |
| PD-1.3 A/G | 7146 | intron 4 | Analyzed here |
| PD-1.4 A/G | 7499 | intron 4 | In full linkage disequilibrium |
| | | | with SNP PD-1.5 |
| PD-1.9 A/G | 7625 | exon 5, Ala- | Frequency <1% in Europeans |
| | | Val | |
| PD-1.5 C/T | 7785 | exon 5, Ala- | Analyzed here |
| | | Val | |
| PD-1.6 A/G | 8738 | 3′ UTR | Analyzed here |

In total 2510 individuals representing five different sets were studied, three of them of European origin: set I, Nordic multicase families (Iceland, Sweden, Norway) ^{9,10}; set II, Swedish trios and sporadic patients; set III, European-American multicase families. The two other sets represented non-European populations: a set of Mexican multi- and single case families and sporadic patients, and a set of African-American multicase families (Table 3).

TABLE 3
Structure of SLE Replication Sets

| | | Number of | Number of |
|---|---|---|---|
| Sets | Structure | Families | Individuals |
| Set I | Nordic multicase families | 31 | 105 |
| Set II | Swedish singlecase families | 66 | 238 |
| | Swedish sporadic patients | | 200 |
| Set III | European-American | 151 | 849 |
| | multicase families | | |
| Set IV | Mexican multicase families | 25 | 129 |
| | Mexican singlecase families | 86 | 279 |
| | Mexican sporadic patients | | 320 |
| Set V | African-American multicase families | 82 | 390 |
| Total | | | 2510 |

Three SNPs were infrequent in Europeans (<1%) and not useful for this study, as well as SNP PD-1.4, which was in complete linkage disequilibrium with SNP PD-1.5 (Table 2). Therefore the present inventors analysed the remaining 3 SNPs in all sets. Affected family-based controls (AFBAC)⁴¹ representing truly never-transmitted-to-the-patients parental chromosomes in the multi- and singlecase families were used as control groups for the corresponding sets. Allele A of an SNP PD-1.3 showed association with SLE in Europeans, p-10 0.00001, RR=2.6 (95% C.I. 1.6-4.4) and Mexicans, p=0.0009, RR=3.5 (95% C.I. 1.4-8.5) (Table 4).

TABLE 4
Distribution of Alleles of SNPs in the PD-1 Gene in Replication Sets

| | | SLE patients | | AFBAC group | | | RR |
|---|---|---|---|---|---|---|---|
| Set | SNP/allele^{a} | n/N^{b} (%) | | n/N (%) | | p value | (95% Cl) |
| European set I: | PD-1.3A | 16/64 | (25) | 3/64 | (5) | 0.0009 | 5.3 (1 .6-17.4) |
| Nordic multicase | PD-1.5C | 42/64 | (66) | 35/64 | (55) | n.s. | — |
| families | PD-1.6A | 8/64 | (13) | 12/64 | (19) | n.s. | — |
| European set II: | PD-1.3A | 56/526 | (11) | 5/132 | (4) | 0.005 | 2.8 (1.1-6.9) |
| Swedish singlecase | PD-1.5C | 316/526 | (60) | 66/132 | (50) | 0.008 | 1.2 (1.0-1.4) |
| Families and | PD-1.6A | 44/526 | (8) | 6/132 | (5) | n.s. | — |
| sporadic patients | | | | | | | |
| European set III: ^{c} | PD-1.3A | 32/290 | (11) | 8/160 | (5) | 0.01 | 2.2 (1.0-4.7) |
| European-American | PD-1.5C | 165/290 | (57) | 84/160 | (53) | n.s. | — |
| multicase families | PD-1.6A | 32/290 | (11) | 30/160 | (19) | 0.009 | 0.6 (0.4-0.9) |
| European sets | PD-1.3A | 104/880 | (12) | 16/356 | (5) | 0.00001 | 2.6 (1.6-4.4) |
| I, II, III: | PD-1.5C | 523/880 | (59) | 185/356 | (52) | 0.003 | 1.1 (1.0-1.3) |
| Summary | PD-1.6A | 84/880 | (10) | 48/356 | (14) | 0.01 | 0.7 (0.5-1.0) |
| Set IV: Mexican | PD-1.3A | 58/804 | (7) | 5/240 | (2) | 0.0009 | 3.5 (1.4-8.5) |
| multi - and | PD-1.5C | 439/804 | (54) | 141/240 | (59) | n.s. | — |
| singlecase families | PD-1.6A | 338/712 | (48) | 107/234 | (46) | n.s. | — |
| and sporadic | | | | | | | |
| patients | | | | | | | |
| Set V: African- ^{c} | PD-1.3A | 5/160 | (3) | 0/24 | (0) | n.s. | — |
| American multicase | PD-1.5C | 71/160 | (42) | 15/24 | (63) | 0.05 | 0.7 (0.5-1.0) |
| families | PD-1.6A | 54/160 | (39) | 7/24 | (29) | n.s. | — |

Abbreviations: AFBAC—affected family-based controls, RR—relative risk; a) one allele of each SNP is shown to simplify the table; b) n/N=number of alleles out of total number of chromosomes; c) one patient was randomly selected from each multicase family; n.s. denotes not significant. Remarkably, PD-1.3.A was less frequent in Mexicans than in Europeans and almost not present in African-Americans, suggesting that this mutation is of recent origin affecting mostly Europeans and to a lesser extent populations admixed with them. Alleles of SNPs PD-1.5 and PD-1.6 demonstrated residual association with SLE due to linage disequilibrium with SNP PD-1.3, but no increase in the relative risk (Table 4).

The inventors found that PD-1.3 is located in an enhancer-like structure in intron 4 of the PD-1 gene, where four imperfect tandem repeats contain binding sites for transcription factors exclusively involved in hematopoietic differentiation and inflammation: AML-1, E box-binding factors and NFκB (p50)^{29,31}. The SNP PD-1/3A disrupts the predicted DNA-binding site for AML-1 in the first repeat (FIG. 22). In FIG. 22 the predicted binding sites for the transcription factors AML-1, NFκB (p50) and E-box-binding factors are shown. The first AML-1 binding site is disrupted by SNP PD1.3A, hence, the predicted binding sites in the intronic enhancer are within the PD-1 gene. AML-1, also known as CBFα2, is a transcription factor inactivated by translocations found in acute myeloid leukaemia, and is shown to either repress or activate transcription²⁹. The inventors show here the specific binding of nuclear extract from a human T cell-line, Jurkat, to the wild-type AML-1 binding site (PD-1.3, allele G), confirmed by supershifting upon addition of antibodies against AML-1. No binding was found to the mutated site (PD-1.3, allele A) at any concentration of the nuclear extract (FIG. 23). FIG. 23 shows the 18 bp oligonucleotides containing both allelic variants of SNP PD-1.3 which represent native and mutated AML-1 binding sites. Binding sites were assayed with increasing (0-4 μg) amounts of Jurkat cells nuclear extract. Lanes 1-4 show that there is a lack of binding to oligonucleotide containing PD-1.3A; lanes 5-12 show that there is binding to native PD-1.3G containing oligonucleotide. Binding to wild-type G allele results in a complex (C) that is specifically competed by 100× excess of unlabelled PD-1.3 G oligonucleotide (lane 11) but not by the same amount of unrelated oligonucleotide (lane 12). Antiserum against AML-1 reveals a supershifted band (S) as indicated in lane 9, while unrelated serum does not produce the supershift as indicated in lane 10.

To evaluate the enhancer activity of the intronic sequences surrounding SNP PD-1.3, both allelic forms of SNP PD-1.3 were cloned into the enhancer position of the pGL3-promoter vector and transfected into Jurkat cells. The level of expression was determined with a Luciferase reporter gene assay. The presence of allele A enhanced the basal transcriptional activity of the reporter gene (p-0.0006). Activation of the cells by PMA and ionomycin resulted in a 8-fold increase for the wild-type allele G construct (p=0.0004), while only in a 1.3-fold increase in the presence of allele A (FIG. 24). FIG. 24 shows the results of three independent transfections performed in duplicate. Activation by PMA and ionomycin was performed after 2 hours after transfection. Cells were activated for 8 hours without visual damage. Luciferase activity was measured at 10 hours after transfection and the results normalised with β-gal control. Results of relative Luciferase expression are shown as bars. Constructs containing allele A in non-activated Jurkat cells showed a higher level of basal expression than those containing allele G (p=0.0006). Activation of the Jurkat cells with PMA+lonomycin resulted in an increase of Luciferase expression by 8.3 fold (p=0.0004) for a construct with allele G and only by 1.3-fold for a construct with allele A of SNP PD1.3. Therefore the inventors prove that the intronic sequence containing SNP PD-1.3 has an enhancer activity and that the A/G allelic variation of this SNP produces a missregulation of the cellular response upon PMA and ionomycin activation.

The inventors then determined whether PD-1 expression is altered in SLE patients compacted to controls. We studied the PD-1 expression profiles in 17 healthy females homozygous for PD-1.3G in non-activated peripheral blood mononuclear cells (PBMC) and after 2 and 4 hours of activation by PMA and ionomycin. The inventors found a uniform pattern of expression in all control samples with a sharp increase at 2 hours after activation, (p=0.0001) and further increase at 4 hours (FIG. 25). FIG. 25 shows the results of PBMC of 17 controls with SNP PD-1.3G/G genotypes, 8 patients with SNP PD-1.3G/G genotypes, 4 patients with SNP PD-1.3A/G genotypes and one patient with SNP PD-1.3A/A. Genotype were activated with MPA & ionomycin for 2 and 4 hours and compared with untreated samples. Untreated samples are marked as 1, activated for 2 hours as 2 and activated for 4 hours as 3. Level of PD-1 mRNA normalized by β2-microglobulin was measured in triplicate using TaqMan technology. The Δ Ct value is derived as a difference between Ct_{PD-1} and Ct_{β2-microglobulin} values. High Δ Ct value means low PD-1 expression and vice versa. Results of PD-1 mRNA expression are presented as box-and-whiskers plots. Black bars represent 50% of sample distribution and whiskers show high and low extremes of distribution. SLE patients, particularly those positive for allele A of SNP PD-1.3 show higher degree of variation. *-indicates higher (p=0.006) level of PD-1 expression in unactivated cells of patients positive for allele A of the SNP PD-1.3 than in unactivated cells of controls. PD-1 expression in SLE patients had much higher degree of inter-individual variation than in controls. Level of PD-1 expression in patients homozygous for SNP PD-1.3G (8 individuals) was also higher at 2 hours after activation, (p=0.05). Patients heterozygous for the allele PD-1.3A (4 individuals) together with a patient with a very rare genotype PD-1.3 A/A (frequency <1% in SLE patients and 0% in controls) had higher basal expression of PD-1 than controls (p=0.006) and diverse response to activation at 2 hours (FIG. 25). These results resemble those obtained in the Luciferase reporter system. Therefore, allele A of the SNP PD1.3 influences the expression of PD-1 in SLE patients as well as in controls.

The results from these experiments suggest that under normal conditions the AML-1 transcription factor binds to the PD-1 wild-type enhancer and represses transcription of the PD-1 gene. The role of other transcription factors such as NFκB (p50) and E box-binding factors in the regulation of PD-1 in health and disease conditions remains to be established. Upon cellular activation the wild-type enhancer provides a rapid increase in PD-1 expression, and PD-1 being an immunoreceptor tyrosine-based inhibitory motif-containing molecule (ITIM) ^{11,12,14,17}, inhibits autoreactive cells and preserves self-tolerance^{42,43}. However, disruption of the AML-1 binding site by SNP PD-1.3A leads to de-repression of the basal transcription of PD-1 and an inability for the mutated enhancer to provide an adequate response upon cell activation, suggesting a defect in the on-off switch affecting PD-1 expression. Perhaps, aberrant regulation of PD-1 leads to disregulation self-tolerance and to the chronic lymphocyte hyperactivity characteristic of SLE²²⁻²⁴. These results give new insights to the understanding of the disease and may help to improve its diagnosis and treatment.

Methods

Family Material

The 31 Nordic multicase families, from Iceland, Sweden and Norway have been previously described^{9,10}. Two sets of sporadic Swedish SLE patients and their families as well as their controls were obtained and carefully characterised as to Swedish ancestry (determined for at least 2 generations through a questionnaire) and clinical manifestations. All patients fulfilled the American College of Rheumatology Classification Criteria for SLE³⁵. One set of patients was from Southern Sweden and the second from Mid-Sweden. Controls were from the same geographical locations. Of the patients, family members were available and haplotypes could be constructed for 190. A total of 454 individuals were used or 2.5 relatives/patient. A smaller group of 64 patients were also included belonging to both sets, but these had no family members available. Thus, a total of 254 sporadic patients were studied. The data for both sets was first analysed separately and then combined as shown in Table 1. All patients and controls used were females because of the bias towards women in SLE (9:1).

In patients with kidney involvement, glomerulonephritis was documented clinically by urinalysis and kidney function tests and verified by kidney biopsy. Disease activity was defined by the activity index SLEDAI³⁶. Patients used for the expression analysis had an SLEDAI of 0. Of these, nine patients were treated with low dose prednisolone (ranging from 12,5 mg/day for one patient to 0,5 mg/day) and azathioprine or an antimalarial (100 mg/day and 160 mg/day, respectively) or only with azathioprine or antimalarial (cloroquine). Four patients had no treatment. No patient was treated by cytostatics, known to affect gene expression, and there was no correlation between having treatment and nephritis.

Physical Map

Dr. Graeme Bell kindly provided a physical map of 2q37.3, prior to publication¹⁸. Dr. Patrick Concannon also provided a preliminary physical map (unpublished). The described clones in those maps were obtained from Research Genetics and tested by PCR for the PD-1 gene. Further search for PD-1 in BAC and PAC libraries was attempted through services provided commercially covering all available libraries (Research Genetics and Incyte Genomics).

Fluorescent In Situ Hybridisation (FISH)

The BAC clone RP11-463B12, representing contig AC025684.00001 at the location described in www.ensemble.org, was used as a FISH probe. The Scaffold x54KRCE619J from Celeras' February frozen dataset (www.celera.com) was found to contain fragments of the ESTs L16991 (gene human thymidylate kinase, HTHYK) and AB023160 (mRNA for the KIAA0943 gene) from the public database at NCBI (www.ncbi.nim.nih.gov). The present inventors aligned these sequences to generate a joint FISH probe covering both genes as they were very close to each other (about 2,5 kb apart). The joint FISH probe was made of 6 PCR fragments (free from repetitive elements) of 9.0 kb of total length, covering about 20 kb of genomic sequence. The FISH probe for PD-1 was generated by PCR from the genomic sequence obtained by us and included two PCR fragments of 1.2 kb and 6.7 kb covering almost the entire gene.

FISH with metaphase and interphase chromosomes was performed essentially as described elsewhere³⁷. DNA from the BAC clones was labelled either with biotin or digoxigenin using nick translation. The probes were detected by the application of a single layer of FITC-avidin (Vector labs) and rhodamine labelled anti-digoxigenin antibodies (Boehringer-Mannheim). Images were merged using a Zeiss Axioscope microscope with a cooled CCD camera (Photometrics) and the IPlab software (Vysis).

Genotyping of the SNPs

The complete PD-1 gene was sequenced using the dye-terminator kit following the manufacturer's instructions (PE Biosystems). The SNPs for M64098 (HDLBP), D63878a (NEDD5), AA15760 (Cda0fd11) and AB023160 (KIM0943) were discovered by in silico search and sequencing and have been previously described¹¹. Sequences for the polymorphisms UCSNP-6,-11,-12,-15 and -19 were kindly provided by Prof. Graeme Bell. The SNPs were genotyped by restriction enzyme analysis (RFLP) or dynamic allele-specific hybridisation (DASH)³⁸ or PCR followed by agarose gel analysis (for the UCSNP-19 minisatellite).

The following primers and restriction enzymes were used for PD-1: PD1.2, DASH assay: PD1.2f: 5′ CTG CAT CTG GGG GAA TGG TGA C 3′, PD1.2r: 5′ GAT TCC AGA GCT AGA GGA CAG A 3′-biotin, PD1.2probe1: 5′ GGT GAC CGG CAT CTC 3′, PD1.2probe2: 5′ GGT GAC CAG CAT CTC 3′. PD1.3f: 5′ CCC CAG GCA GCA ACC TCA AT 3′, PD1.3r: 5′ GAC CGC AGG CAG GCA CAT AT 3′, 180 bp (130+50) PstI. For DASH: DPD1.3f: 5′ TGG TGC CCC AGC CCA CCT G 3′, DPD1.3r: 5′ CAT GGG ACT GGC ACC CCC GGA 3′-biotin and as PD1.3 probe: 5′CAC CTG CGG TCT CCG 3′. For PD1.5: PD1.5f: 540 CTC AAA GM GGA GGA CCC CTC A 3′, PD1.5r: 5′ GCC MG AGC AGT GTC CAT CCT 3′, 240 bp (180+60), PvuII, and for PD1-6, PD1.6f: 5′ CAT CCT ACG GTC CCA AGG TCA 3′, PD1.6r: 5′ TGT GTG GAT GTG AGG AGT GGA TAG 3′, 267 bp (153+114), NdeI. All primers and probes were synthesized by Interactiva (Interactiva Division, ThermoHybaid).

Transcription Factor Binding Site Identification

We used the TFSEARCH database to predict potential transcription factor binding sites in the different promoter and intronic sequences of PD-1.

Electrophoretic Mobility Shift and Supershift Assays

Nuclear extracts from Jurkat T cells was prepared according to established methodology³⁹. EMSA was performed using 32P-labelled ds-oligonucleotides (10 fmole) with the same specific activity for PD1.3G: 5′ gat ctC CCA CCT GCG GTC TCC GG 3′ and PD1.3A: 5′ gat ctC CCA CCT GCA GTC TCC GG 3′, in an DNA-binding reaction [2 mM HEPES (pH 7.9), 10 mM Tris-HCl (pH 7.5), 25 mM NaCl, 10 mM KCl, 1.5 mM EDTA, 0.1 mM ZnSO₄, 15% glycerol, 0.25 mg/ml BSA, 0.6 mM DTT, 2 μg poly(dI·dC)] for 20 min on ice, separated on 6% PAGE and visualized by autoradiography. For specific and unspecific competition unlabelled PD1.3G and an unrelated oligonucleotide were used at 100-fold molar excess. Polyclonal goat-anti-AML-1 antisera (1 μg) and matched control antisera was obtained from Santa Cruz Biotechnology (Santa Cruz, Calif.).

Luciferase Reporter Gene Assay

The complete intron 4 (560 bp) sequence from the PD1.3A or PD1.3G alleles were amplified by PCR and the fragments were cloned into the BamHI site of a pGL3-promoter vector containing the SV40 viral promoter (Promega). Jurkat cells (3×10⁶) were transiently transfected with 0.4 μg of the pGL3 constructs using Effectene (Qiagen, Valencia, Calif.). Co-transfection by 0.1 μg of β-actin-LacZ reporter was used to correct luciferase values for differences in transfection efficiency. Luciferase expression was analysed in a luminometer (Lumat LB9501, EG&G Berthold, Bad-Wildbad, Germany), using Luciferase Assay Reagent (Promega). Cells were activated by a combination of PMA (phorbol-12-myristate-13-acetate, 20 ng/ml) and lonomycin (0.5 μM) (Sigma) for 10 hours. Cloning of the intron in either orientation showed identical results.

Expression of PD-1 Using Real Time PCR

Fresh peripheral blood mononuclear cells (PBMC) samples from 17 healthy women and 13 female patients with SLE were cultured for 0, 2 and 4 hours with PMA+lonomycin (20 ng/ml and 0,5 μM, respectively) or left untreated. Cells were harvested and total RNA was prepared using the Trizol reagent (Life Technologies) and standard methods. cDNA was prepared with random hexamers using the TaqMan Reverse transcription reagents Kit (PE Biosystems). The primers and probes were designed using the Primer Express software (PE Biosystems). The primers and probes used were: PD1.TaqManF:5′ CCA GCC CTG MG GAG GA 3′. PD1TaqManR: 5′ MT CCA GCT CCC CAT AGT CCA 3′ and the PD1 probe was: 5′FAM-AGA GM CAC AGG CAC GGC TGA GGG-TAMRA 3′. 2 nM MgCl₂, 40 nM of the probe and of each primer together with the TaqMan PCR Core Reagents kit (PE Biosystems) were used for the PD-1 assay. Human α2-microglobulin (PE Biosystems) was used as an endogenous control as recommended. Samples were run and analysed in the Sequence Detection system, ABI Prism 7700 (PE Biosystems). Samples were run in triplicates from which the mean was calculated. Ct values were used to determine differences in the expression of PD-1 and α2-microglobulin, and then converted into x-folds.

Statistical Analysis

Linkage analysis was performed using the MLINK routine of the ANALYZE software as described previously^{9,10} and developed by Joseph Terwilliger⁴⁰. All linkage analyses were performed using an “affected-only” analysis, with a dominant mode of inheritance and a disease gene frequency of 0.002. Association analysis of the major haplotype with SLE in the multicase and single case families (trios) was performed using the TDT routine of ANALYZE. Otherwise association was tested with 2×2 contingency tables and X² analysis. P values were calculated using the Fisher's exact test. Multiple testing was conservatively corrected with the Bonferroni method.

Accession Numbers and Databases

The complete sequence of the PD-1 gene has been deposited in GeneBank with the accession number AF363458. The databases used were www.ensemble.org and ncbi.nim.nih.gov, www.celera.com and (http://molsun1.cbrc.aist.go.jp/research/db/TFSEARCH.html.

Families, Patients and Control

The inventors studied 31 Nordic multicase families described previously^{9,10}, 25 Mexican multicase families, 151 European-American and 82 African-American multicase families, 66 Swedish singlecase families and additionally 200 Swedish sporadic patients, 89 Mexican singlecase families and additionally 320 Mexican sporadic female patients, 2510 individuals in total. Only female patients in were studied in all sets. All patients fulfilled the American College of Rheumatology's Criteria for SLE³⁵. As a control group untransmitted parental chromosomes (AFBAC) in multi- and single case families were used.

Fluorescent In Situ Hybridization (FISH)

FISH for the PD-1 gene (10 kb) and for a joint probe for the ESTs L16991 and AB023160 (20 kb) on metaphase and interphase chromosomes was performed as described³⁷.

Sequencing and Genotyping of the SNPs

The complete PD-1 gene (9.6 kb) was sequenced and 7 SNPs were detected and genotyped by RFLP, DASH adapted for FRET signal generation^{38,44} or sequencing (primers and probes available upon request).

Transcription Factor Binding Site Identification

TFSEARCH database (http://molsun.1.cbrc.aist.go.jp/research/db/TFSEARCH.html) was used for binding site predication.

Electrophoretic Mobility Shift and Supershift Assays

Nuclear extracts from Jurkat T cells was prepared as described³⁹. EMSA was performed using ³²P-labelled ds-oligonucleotides (10 fmole) with the same specific activity for PD1.3G:5′ gat ctC CCA CCT GCG GTC TCC GG3′ and PD1.3A: 5′ gat ctC CCA CCT GCA GTC TCC GG3′, in a DNA-binding reaction [2 mM HEPES (pH 7.9), 10 mM Tris-HCl (pH 7.5), 25 mM NaCl, 10 mM KCl, 1.5 mM EDTA, 0.1 mM ZnSO₄, 15% glycerol, 0.25 mg/ml BSA, 0.6 mM DTT, 2 μg poly(dI.dC)] for 20 min on ice, separated on 6% PAGE and visualized by autoradiography. For competition, unlabelled PD1.3G and an unrelated oligonucleotide were used at 100-fold molar excess. Polyclonal goat-anti-AML-1 antiserum (1 μg) and matched unrelated antiserum were obtained from Santa Cruz Biotechnology (Santa Cruz, Calif.).

Luciferase Reporter Gene Assay

The complete intron 4 (560 bp) containing allele PD1.3A or PD1.3G was cloned into a BamHI site of the pGL3 promoter vector (Promega). Jurkat cells (3×10⁶) were transiently co-transfected with 0.4 μg of the pGL3 constructs and 0.1 μg of β-actin-LacZ reporter using Effectene (Qiagen, Valencia, Calif.). Cells were activated by a combination of PMA (phorbol-12-myristate-13-acetate, 20 ng/ml) and lonomycin (0.5 μM) (Sigma) for 10 hours

Expression of PD-1 Using RT-PCR

Thirteen female patients with inactive stage of SLE—a disease activity index SLEDAI of 0 (ref 36), (nine with low dose treatment, four with no treatment) and 17 healthy women matched by age were genotyped for SNP PD-1.3. Fresh peripheral blood mononuclear cells (PBMC) samples from all individuals were cultured for 0, 2 and 4 hours with or without PMA+lonomycin (20 ng/ml and 0.5 μM, respectively). cDNA was prepared with random hexamers and PD-1 expression was evaluated on TaqMAn (ABI 7700 and SDS software, PE Biosystem). Expression was normalized by Human β2-microglobulin (PE Biosystems) and all samples were run in triplicates.

Primers and probes were designed to cover exon-exon borders in cDNA and therefore amplification cannot be achieved from genomic DNA. The primers and probes were: PD1.TaqManF:5′ CCA GCC CTG MG GAG 3′. PD1TaqManR: 5′ AAT CCA GCT CCC CAT AGT CCA 3′ and the PD1 probe was: 5′FAM-AGA GM CAC AGG CAC GGC TGA GGG-TAMRA 3′. PCR was run as recommended (PE Biosystems) with 2 nM MgCl₂, and 40 nM of each primers and probe.

Statistical Analyzis

AFBAC analysis was performed as described⁴¹. Alleles of all SNPs were confirmed to be in Hardy-Weinberg equilibrium in non-affected sibs in all families (no transmission distortion was observed). Association was tested with 2×2 contingency tables and X² analysis. Relative risk (RR) was calculated with 95% confidence intervals. Expression and transfection assays were analysed with an unpaired Student's t-test.

References

1. Gudmundsson S and Steinsson K. Systemic lupus erythematosus in Iceland 1975 through 1984. A nationwide epidemiological study in an unselected population. J Rheumatol 17, 1162-1167 (1990).

2. Lawrence R C et al. Estimates of the prevalence of arthritis and selected musculoskeletal disorders in the United States. Arthritis Rheum 41,778-99 (1998).

3. Stahl-Hallengren C, Jonsen A, Nived O and Sturfelt G. Incidence studies of systemic lupus erythematosus in Southern Sweden: increasing age, decreasing frequency of renal manifestations and good prognosis. J Rheumatol 27, 685-691 (2000).

4. Arnett F C and Reveille J D. Genetics of systemic lupus erythematosus. Rheum Dis Clin North Am 18, 865-92 (1992).

5. Moser K L et al. Genome scan of human systemic lupus erythematosus: evidence for linkage on chromosome 1q in African-American pedigrees. Proc Natl Acad Sci USA 95,14869-14874 (1998).

6. Gaffney P M et al. A genome-wide search for susceptibility genes in human systemic lupus erythematosus sib-pair families. Proc Natl Acad Sci USA 95,14875-14879 (1998).

7. Gaffney P M et al. Genome screening in human systemic lupus erythematosus: results from a second Minnesota cohort and combined analyses of 187 sib-pair families. Am J Hum Genet 66, 547-566 (2000).

8. Shai R et al. Genome-wide screen for systemic lupus erythematosus susceptibility genes in multiplex families. Hum Mol Genet 8,639-644 (1999).

9. Lindqvist A K et al. A susceptibility locus for systemic lupus erythematosus (hSLE1) in chromosome 2q. J Autoimm 14,169-178 (2000).

10. Magnusson V et al. Fine mapping of the SLEB2 locus involved in susceptibility to systemic lupus erythematosus Genomics 70, 307-314 (2000).

11. Shinohara T, Taniwaki M, Ishida Y, Kawaichi M and Honjo T. Structure and chromosomal localization of the human PD-1 gene (PDCD1). Genomics 23,704-706 (1994).

12. Finger L R et al. The human PD-1 gene: complete cDNA, genomic organization, and developmentally regulated expression in B cell progenitors. Gene 197,177-187(1997).

13. Nishimura H, Honjo T and Minato N. Facilitation of □ selection and modification of positive selection in the thymus of PD-1-deficient Mice. J Exp Med 191, 891-897 (2000).

14. Vibhakar R, Juan G, Traganos F, Darzynkiewicz Z and Finger L R. Activation-induced expression of human programmed death-1 gene in T-lymphocytes. Exp Cell Res 232, 25-28 (1997).

15. Freeman G J et al. Engagement of the PD-1 immunoinhibitory receptor by a novel B7 family member leads to negative regulation of lymphocyte activation. J Exp Med 192, 1027-1034 (2000).

16. Latchman Y et al. PD-1 L2 is a second ligand for PD-1 and inhibits T cell activation. Nat Immunol 2, 261-268 (2001).

17. Nishimura H, Nose M, Hiai H, Minato N and Honjo T. Development of lupus-like autoimmune diseases by disruption of the PD-1 gene encoding an ITIM motif-carrying immunoreceptor. Immunity 11,141-151 (1999).

18. Horikawa Y et al. Genetic variation in the gene encoding calpain-10 is associated with type 2 diabetes mellitus. Nat Genet 2,163-175 (2000).

19. Ghozi M C, Bernstein Y, Negreanu V, Levanon D and Groner Y. Expression of the human acute myeloid leukemia gene AML1 is regulated by two promoter regions. Proc Natl Acad Sci (USA) 93, 1935.1940 (1996).

20. Lander E S et al. Initial sequencing and analysis of the human genome. Nature 409, 860-921 (2001).

21. Venter J C et al. The sequence of the human genome. Science 291,1304-1351 (2001).

22. Singh T et al. Evidence for multiple mechanisms of polyclonal T cell activation in murine lupus. J Clin Invest 102,1841-1849 (1998).

23. Estess P, DeGrendele H C, Pascual V and Siegelman M H. Functional activation of lymphocyte CD44 in peripheral blood is a marker of autoimmune disease activity. J Clin Invest 102, 1173-1182 (1998).

24. Fernandez-Gutierrez B, de Miguel S, Morado C, Hernandez-Garcia C, Banares A, Jover J A. Defective early T and T-dependent B cell activation in systemic lupus erythematosus. Lupus 7, 314-322 (1998).

25. Bolland, S and Ravetch, J V. Spontaneous autoimmune disease in FcGRIIB-deficient mice results from strain-specific epistasis. Immunity 13, 277-285 (2000).

26. Mary C et al. Dysregulated expression of the Cd22 gene as a result of a short interspersed nucleotide element insertion in Cd22a lupus-prone mice. J Immunol 165, 2987-2996 (2000).

27. Salmon J E et al. Fc gamma RIIA alleles are heritable risk factors for lupus nephritis in African Americans. J Clin Invest 97,1348-1354 (1996).

28. Wu J et al. A novel polymorphism of FcgammaRIIIa, (CD16) alters receptor function and predisposes to autoimmune disease. J Clin Invest 100,1059-1070 (1997).

29. Lutterbach B and Hiebert S W. Role of the transcription factor AML-1 in acute leukemia and hematopietic differentiation. Gene 245, 223-235, (2000).

30. Franzoso G et al. Requirement of NFkB in osteoclast and B-cell development. Genes Dev 11, 3482-3496, (1997).

31. Wong H K, Kammer G M, Dennis G and Tsokos G C. Abnormal NF-kappa B activity in T lymphocytes from patients with systemic lupus erythematosus is associated with decreased p65-ReIA protein expression. J Immunol 163,1682-1689 (1999).

32. Duncan D D, Adlam M and Siu G. Asymmetric redundancy in CD4 silencer function. Immunity 4, 301-311 (1996).

33. Makar K W, Pham C T N, Dehoff M H, O'Connor S M, Jacobi S M and Holers V M. An intronic silencer regulates B lymphocyte cell-and stage-specific expression of the human complement receptor type 2 (CR2, CD21) gene. J Immunol 160, 1268-1278 (1998).

34. Bruhn L, Munnerlyn A and Grosschedl R. A L Y, a context-dependent coactivator of LEF-1 and AML-1, is required for TCRalpha enhancer function. Genes Dev 11, 640-653 (1997).

35. Tan E M et al. The 1982 revised criteria for the classification of systemic lupus erythematosus. Arthritis & Rheum 25, 1271-1277 (1982).

36. Bombardier C, Gladman D D, Urowitz M B, Caron D and Chang C H. Derivation of the SLEDAI—a disease activity index for lupus patients. Arthritis Rheum 35, 630-640 (1992).

37. Lichter P, Cremer T, Borden J, Manuelidis L and Ward D C. Delineation of individual human chromosomes in metaphase and interphase cells by in situ suppression hybridization using recombinant DNA libraries. Hum Genet 80, 224-234 (1988).

38. Howell W M, Jobs M, Gyllensten U and Brookes A J. Dynamic allele-specific hybridization (DASH). Nat Biotechnol. 17, 87-8 (1999).

39. Andrews, N C and Faller, D V. A rapid micropreparation technique for extraction of DNA-binding proteins from limiting numbers of mammalian cells. Nucleic Acids Research 19,2499 (1991).

40. Terwilliger J D. A powerful likelihood method for the analysis of linkage disequilibrium between trait loci and one or more polymorphic marker loci. Am J Hum Genet 56, 777-87 (1995).

41. Thomson, G. Mapping disease genes: family based association studies. Am J Hum Genet, vol. 57 pp.487-498.

42. Nishimura, H. and Honjo, T. PD-1: an inhibitory immunoreceptor involved in peripheral tolerance. Trends Immunol 22(5), 265-8 (2001).

43. Goodnow, C. C., Pathways for self-tolerance and the treatment of autoimmune diseases. Lancet 357(9274), 2115-21 (2001).

44. Prince, J. A., Feuk, L., Howell, M., Jobs, M., Emahzion T. and Brookes, A. J. Robust and Accurate Single Nucleotide Polymorphism Genotyping by Dynamic Allele-Specific Hybridization (DASH): Design Criteria and Assay Validation. Genome Res 11, 152-162 (2001).

1. An isolated nucleic acid encoding a polymorphic region of PD-1, characterised in that the nucleic acid sequence comprises sequentially;
a) A promoter region,
b) a first exon,
c) a first intron,
d) a second exon,
e) a second intron,
f) a third exon,
g) a third intron,
h) a fourth exon,
i) a fourth intron,
j) a fifth exon and,
k) a 3′UTR,
or a sequence complementary thereto.

2. A nucleic acid according to claim 1, which encodes a mammalian PD-1, or a sequence complementary thereto.

3. The nucleic acid of claim 1 or claim 2, which encodes a human PD-1, or a sequence complementary thereto.

4. A nucleic acid according to claim 1, in which the nucleic acid comprises a polynucleotide having at least 80% nucleotide identity with the sequence located between position 1 and position 9625 of the nucleotide sequence of SEQ ID N^{o} 1, fragments thereof, or a sequence complementary thereto.

5. An isolated nucleic acid in which the nucleic acid comprises a polynucleotide having at least 80% nucleotide identity with any one of the nucleotide sequences of SEQ ID N^{o}s 2-12, fragments thereof, or a complementary sequence thereto.

6. An isolated polynucleotide fragment of a nucleic acid according to any one of claims 1 to 5, in which the fragment comprises at least 10 nucleotides of PD-1.

7. An expression product of the nucleic acids of any one of claims 1 to 6.

8. A pharmaceutical composition comprising any one of the nucleic acids or expression products thereof of claims 1-6.

9. Use of a composition according to claim 7 for the treatment of mammals.

10. Use of a composition according to claim 7 for the treatment of humans.

11. Use of a composition according to any one of claims 7 to 9 in medicine.

12. Use of a composition according to any one of claims 7, 8 or 9 for the treatment or alleviation of autoimmune disorders.

13. Use according to claim 12, in which the autoimmune disorder is multiple sclerosis, myasthenia gravis, Type 1 diabetes, rheumatoid arthritis, Sjögrens syndrome, atopy or allergy.

14. Use according to claim 13, in which the autoimmune disorder is systemic lupus erythematosus.

15. Use according to any one of claims 12 to 14, in which the autoimmune disorder is characterised by conditions selected from the group including any one or more of;
fatigue, fever, loss of appetite, nausea, weight loss, hives, loss of scalp hair, red “butterfly rash” and raised rash, sensitivity to sun, ulcers in mouth, nose, or vagina, arthritis, joint pain, loss of blood supply to bone, pain, infections within joints, decrease in kidney function including, blood, aberrant amounts of protein or white blood cells in urine, intracerebral haemorrhage, headaches, loss of coordination, memory loss, seizures, strokes, anaemia, low white blood cell or low platelet count, pericardial effusion, heart attack, inflammation in the heart, infection in the heart, inflammation of the lining of the heart, infection of the lining of the heart, heart valve problems, shortness of breath, cough, inflammation of the lungs, inflammation of the lining of the lungs, abdominal distress, diarrhoea, enlargement of the liver, loss of appetite, nausea and vomiting, blindness, visual impairment, dryness of the eyes and dryness of the mouth.

16. Use of any one of the nucleic acids of SEQ ID N^{o}s 1 to 34 , expression products thereof or complementary nucleic acid sequences thereto, in an ex vivo method of diagnosis or prognosis of autoimmune diseases or of determining a predisposition towards an autoimmune disease, where the autoimmune disease is selected from;
a) multiple sclerosis,
b) myasthenia gravis,
c) Type 1 diabetes,
d) rheumatoid arthritis,
e) Sjögrens syndrome
f) atopy,
g) allergy, or
h) systemic lupus erythematosus,
where systemic lupus erythematosus is characterised by conditions selected from the group including any one or more;
fatigue, fever, loss of appetite, nausea, weight loss, hives, loss of scalp hair, red “butterfly rash” and raised rash, sensitivity to sun, ulcers in mouth, nose, or vagina, arthritis, joint pain, loss of blood supply to bone, pain, infections within joints, decrease in kidney function including, blood, aberrant amounts of protein or white blood cells in urine, intracerebral haemorrhage, headaches, loss of coordination, memory loss, seizures, strokes, anaemia, low white blood cell or low platelet count, pericardial effusion, heart attack, inflammation in the heart, infection in the heart, inflammation of the lining of the heart, infection of the lining of the heart, heart valve problems, shortness of breath, cough, inflammation of the lungs, inflammation of the lining of the lungs, abdominal distress, diarrhoea, enlargement of the liver, loss of appetite, nausea and vomiting, blindness, visual impairment, dryness of the eyes and dryness of the mouth.

17. A method of determining if a subject is suffering from or has a predisposition towards an autoimmune disorder selected from; a) multiple sclerosis,
b) myasthenia gravis,
c) Type 1 diabetes,
d) rheumatoid arthritis,
e) Sjögrens syndrome
f) atopy,
g) allergy, or
h) systemic lupus erythematosus,
where systemic lupus erythematosus is characterised by conditions selected from the group including any one or more of;
fatigue, fever, loss of appetite, nausea, weight loss, hives, loss of scalp hair, red “butterfly rash” and raised rash, sensitivity to sun, ulcers in mouth, nose, or vagina, arthritis, joint pain, loss of blood supply to bone, pain, infections within joints, decrease in kidney function including, blood, aberrant amounts of protein or white blood cells in urine, intracerebral haemorrhage, headaches, loss of coordination, memory loss, seizures, strokes, anaemia, low white blood cell or low platelet count, pericardial effusion, heart attack, inflammation in the heart, infection in the heart, inflammation of the lining of the heart, infection of the lining of the heart, heart valve problems, shortness of breath, cough, inflammation of the lungs, inflammation of the lining of the lungs, abdominal distress, diarrhoea, enlargement of the liver, loss of appetite, nausea and vomiting, blindness, visual impairment, dryness of the eyes and dryness of the mouth comprising the steps of;
(1) obtaining from a subject a sample rich in nucleic acid and/or protein,
(2) analysing the sample of step (1) for the level of expression of PD-1 or the PD-1 polymorphism present sample, and
(3) interpreting the analysis of step (2).

18. A method of determining if a subject has an allelic variant of the PD-1 gene, comprising the steps of;
(1) obtaining from a subject a sample rich in nucleic acid and/or protein,
(2) analysing the sample of step (1) for the PD-1 allele, and
(3) the presence of the PD-1 allele is determined from the analysis of the sample in step (2) by hybridisation of one or more probes.

19. A probe of claim 18, selected from any one or more of the nucleic acids of SEQ ID N^{o}s 1 to 34, fragments thereof or complementary sequences thereto or peptide sequences of SEQ ID N^{o}s 35 to 38 or fragment thereof.

20. A probe according to claim 18 or claim 19 in which the probe is labelled with a detectable molecule.

21. A peptide according to any one of the preceding claims where the peptide is modified by: hydroxylation, glycosylation or sulphation.

22. A recombinant vector comprising a nucleic acid according to any one of claims 1-6.

23. A recombinant host cell comprising a nucleic acid according to any one of claims 1-6.

24. A transgenic organism comprising a nucleic acid according to any one of claims 1-6.

25. A method for producing a polypeptide encoded by a nucleic acid according to any one of claims 1-6, where the method comprises steps of:
a) culturing, in an appropriate culture medium, a host cell previously transformed or transfected with a polynucleotide encoding PD-1;
b) harvesting the culture medium with or without cells therein or lysing the host cells, and
c) separating or purifying, from said culture medium, or from the cell lysate, the thus produced polypeptide of interest.

26. A method according to claim 25 in which the lysis is performed by sonication or osmotic shock.

27. A method for screening ligand substances or molecules that are able to bind to a PD-1 for the treatment of autoimmune disorders where the disease is selected from;
a) multiple sclerosis,
b) myasthenia gravis,
c) Type 1 diabetes,
d) rheumatoid arthritis,
e) Sjögrens syndrome
f) atopy,
g) allergy, or
h) systemic lupus erythematosus,
said method comprising:
(a) contacting the ligand with a PD-1 or a fragment thereof;
(b) contacting the medium containing the ligand and the PD-1 or a fragment thereof with a PD-1 substrate and allowing the possible binding of the substrate to the PD-1 or a fragment thereof to occur; and
(c) measuring the eventual binding of the substrate to the PD-1 protein or a fragment thereof.

28. An isolated polypeptide according to any one of SEQ ID N^{o}s 36, 37, 38 or 39, comprising at least 10 consecutive amino acids of a polypeptide encoding a PD-1.

29. Use of a isolated polypeptide encoding PD-1, in which the polypeptide has at least 90% sequence identity with any one of the polypeptides of SEQ ID N^{o} 35, 36, 37 and 38, in the preparation of an antibody, for the treatment or alleviation of autoimmune disorders or diagnosis of autoimmune disorders associated with aberrant PD-1 function.
